SEQUENCE LISTING (71 sequences)

SEQ ID NO: 1 - DNA, 40 bp, Artificial
  Synthetic oligonucleotide
agcgatgcgttcgagcatcgcuggaaggtgggggagagtg

SEQ ID NO: 2 - DNA, 21 bp, Artificial
  Synthetic oligonucleotide
aaaacaacacaaccccaaaaa

SEQ ID NO: 3 - DNA, 43 bp, Artificial
  Synthetic oligonucleotide
agcgatgcgttcgagcatcgcuaaaacaacacaaccccaaaaa

SEQ ID NO: 4 - DNA, 18 bp, Artificial
  Synthetic oligonucleotide
ggaaggtgggggagagtg

SEQ ID NO: 5 - DNA, 44 bp, Artificial
  Synthetic oligonucleotide
agcgatgcgttcgagcatcgcugggttggagagttgtttgtttg

SEQ ID NO: 6 - DNA, 19 bp, Artificial
  Synthetic oligonucleotide
cacatctcccccacctcct

SEQ ID NO: 7 - DNA, 41 bp, Artificial
  Synthetic oligonucleotide
agcgatgcgttcgagcatcgcucacatctcccccacctcct

SEQ ID NO: 8 - DNA, 22 bp, Artificial
  Synthetic oligonucleotide
gggttggagagttgtttgtttg

SEQ ID NO: 9 - DNA, 42 bp, Artificial
  Synthetic oligonucleotide
agcgatgcgttcgagcatcgcutggtggtgttgtttttgtgt

SEQ ID NO: 10 - DNA, 23 bp, Artificial
  Synthetic oligonucleotide
cttaaccctattatctccatctc

SEQ ID NO: 11 - DNA, 45 bp, Artificial
  Synthetic oligonucleotide
agcgatgcgttcgagcatcgcucttaaccctattatctccatctc

SEQ ID NO: 12 - DNA, 20 bp, Artificial
  Synthetic oligonucleotide
tggtggtgttgtttttgtgt

SEQ ID NO: 13 - DNA, 45 bp, Artificial
  Synthetic oligonucleotide
agcgatgcgttcgagcatcgcuggtggttttgaaggattttattg

SEQ ID NO: 14 - DNA, 21 bp, Artificial
  Synthetic oligonucleotide
acccaacaccttccctatcct

SEQ ID NO: 15 - DNA, 43 bp, Artificial
  Synthetic oligonucleotide
agcgatgcgttcgagcatcgcuacccaacaccttccctatcct

SEQ ID NO: 16 - DNA, 23 bp, Artificial
  Synthetic oligonucleotide
ggtggttttgaaggattttattg

SEQ ID NO: 17 - DNA, 46 bp, Artificial
  Synthetic oligonucleotide
agcgatgcgttcgagcatcgcuttttgttttgggatgttgtatttt

SEQ ID NO: 18 - DNA, 20 bp, Artificial
  Synthetic oligonucleotide
cctcatccacccaacacctt

SEQ ID NO: 19 - DNA, 42 bp, Artificial
  Synthetic oligonucleotide
agcgatgcgttcgagcatcgcucctcatccacccaacacctt

SEQ ID NO: 20 - DNA, 24 bp, Artificial
  Synthetic oligonucleotide
ttttgttttgggatgttgtatttt

SEQ ID NO: 21 - DNA, 49 bp, Artificial
  Synthetic oligonucleotide
agcgatgcgttcgagcatcgcutgggtttgtagtgttttagtattgttt

SEQ ID NO: 22 - DNA, 23 bp, Artificial
  Synthetic oligonucleotide
tccaccctactttccctacattc

SEQ ID NO: 23 - DNA, 45 bp, Artificial
  Synthetic oligonucleotide
agcgatgcgttcgagcatcgcutccaccctactttccctacattc

SEQ ID NO: 24 - DNA, 27 bp, Artificial
  Synthetic oligonucleotide
tgggtttgtagtgttttagtattgttt

SEQ ID NO: 25 - DNA, 48 bp, Artificial
  Synthetic oligonucleotide
agcgatgcgttcgagcatcgcuttgttttgggatattttatttgtttt

SEQ ID NO: 26 - DNA, 21 bp, Artificial
  Synthetic oligonucleotide
aaaaactccaccctactttcc

SEQ ID NO: 27 - DNA, 43 bp, Artificial
  Synthetic oligonucleotide
agcgatgcgttcgagcatcgcuaaaaactccaccctactttcc

SEQ ID NO: 28 - DNA, 26 bp, Artificial
  Synthetic oligonucleotide
ttgttttgggatattttatttgtttt

SEQ ID NO: 29 - DNA, 43 bp, Artificial
  Synthetic oligonucleotide
agcgatgcgttcgagcatcgcugaggggaggggtgtgaatgtg

SEQ ID NO: 30 - DNA, 22 bp, Artificial
  Synthetic oligonucleotide
cattcctccctactcccaaaaa

SEQ ID NO: 31 - DNA, 44 bp, Artificial
  Synthetic oligonucleotide
agcgatgcgttcgagcatcgcucattcctccctactcccaaaaa

SEQ ID NO: 32 - DNA, 21 bp, Artificial
  Synthetic oligonucleotide
gaggggaggggtgtgaatgtg

SEQ ID NO: 33 - DNA, 42 bp, Artificial
  Synthetic oligonucleotide
agcgatgcgttcgagcatcgcutggtggatgttttgggattt

SEQ ID NO: 34 - DNA, 27 bp, Artificial
  Synthetic oligonucleotide
caacatttctacctctactcccacctt

SEQ ID NO: 35 - DNA, 49 bp, Artificial
  Synthetic oligonucleotide
agcgatgcgttcgagcatcgcucaacatttctacctctactcccacctt

SEQ ID NO: 36 - DNA, 20 bp, Artificial
  Synthetic oligonucleotide
tggtggatgttttgggattt

SEQ ID NO: 37 - DNA, 45 bp, Artificial
  Synthetic oligonucleotide
agcgatgcgttcgagcatcgcugttttggaaggattgagaaatgg

SEQ ID NO: 38 - DNA, 25 bp, Artificial
  Synthetic oligonucleotide
caccctaaccactacataaaacaaa

SEQ ID NO: 39 - DNA, 47 bp, Artificial
  Synthetic oligonucleotide
agcgatgcgttcgagcatcgcucaccctaaccactacataaaacaaa

SEQ ID NO: 40 - DNA, 23 bp, Artificial
  Synthetic oligonucleotide
gttttggaaggattgagaaatgg

SEQ ID NO: 41 - DNA, 48 bp, Artificial
  Synthetic oligonucleotide
agcgatgcgttcgagcatcgcutagggagtatataggttggggaagtt

SEQ ID NO: 42 - DNA, 27 bp, Artificial
  Synthetic oligonucleotide
aacacacaataacaaacacaaattcac

SEQ ID NO: 43 - DNA, 22 bp, Artificial
  Synthetic oligonucleotide
agcgatgcgttcgagcatcgcu

SEQ ID NO: 44 - DNA, 25 bp, Artificial
  Synthetic oligonucleotide
agggagtatataggttggggaagtt

SEQ ID NO: 45 - DNA, 129 bp, Artificial
  Synthetic oligonucleotide
ggaaggtgggggagagtggtttggattttagtattttttttttttttagggttaggttttgtttggttattttttgttgttataggtgtgtttggtatagatatttagtttttggggttgtgttgtttt

SEQ ID NO: 46 - DNA, 128 bp, Artificial
  Synthetic oligonucleotide
gggttggagagttgtttgtttgagttgtattttgttttgttttgttttgttttgatagttttggtggtgaggtgggggttgggagatggggagggtagggttaggtgggggaggaggtgggggagatg

SEQ ID NO: 47 - DNA, 150 bp, Artificial
  Synthetic oligonucleotide
tggtggtgttgtttttgtgtaggatggaaggtgtttttgtggggttaggaggttggatagttgtttgttttagttgtattttgttttgttttgttttaggaggttttggtggtgaggtgggggttgtgagatggagataatagggttaag

SEQ ID NO: 48 - DNA, 80 bp, Artificial
  Synthetic oligonucleotide
ggtggttttgaaggattttattgtgtttggtaatttattgtttatgttagtttgggattaggatagggaaggtgttgggt

SEQ ID NO: 49 - DNA, 125 bp, Artificial
  Synthetic oligonucleotide
ttttgttttgggatgttgtattttttttttgattaggggtggttttgaaggattttattgtgtttggtaatttattgtttatgttagtttgggattaggatagggaaggtgttgggtggatgagg

SEQ ID NO: 50 - DNA, 129 bp, Artificial
  Synthetic oligonucleotide
tgggtttgtagtgttttagtattgttttgggatattttatttgttttttaggtgtgatttgttaataggtttgtattggtgataaaaggagtagttttgaatgtagggaaagtagggtggagttttttg

SEQ ID NO: 51 - DNA, 106 bp, Artificial
  Synthetic oligonucleotide
ttgttttgggatattttatttgttttttaggtgtgatttgttaataggtttgtattggtgataaaaggagtagttttgaatgtagggaaagtagggtggagttttt

SEQ ID NO: 52 - DNA, 119 bp, Artificial
  Synthetic oligonucleotide
gaggggaggggtgtgaatgtgtggatttttgtggagagtggaaatatggggagttgaggggagtatgtgtgggttttagaaagttttgggaaattgatttttgggagtagggaggaatg

SEQ ID NO: 53 - DNA, 59 bp, Artificial
  Synthetic oligonucleotide
agtgttggaggttttgaggttagtttaagttgttttaaaatggaatgaaggtgtttgtg

SEQ ID NO: 54 - DNA, 107 bp, Artificial
  Synthetic oligonucleotide
tggtggatgttttgggatttggtttttttgaaggtgttgggggttggggatggtttaggtagtggtgtaggtgttttaggaaggtgggagtagaggtagaaatgttg

SEQ ID NO: 55 - DNA, 98 bp, Artificial
  Synthetic oligonucleotide
gttttggaaggattgagaaatggggattggttagattaggttgtttagttttttggtttttattgttgttttttttgttttatgtagtggttagggtg

SEQ ID NO: 56 - DNA, 20 bp, Artificial
  Synthetic oligonucleotide
tccatgacgttcctgacgtt

SEQ ID NO: 57 - DNA, 18 bp, Artificial
  Synthetic oligonucleotide
tctcccagcgtgcgccat

SEQ ID NO: 58 - DNA, 30 bp, Artificial
  Synthetic oligonucleotide
accgatgacgtcgccggtgacggcaccacg

SEQ ID NO: 59 - DNA, 24 bp, Artificial
  Synthetic oligonucleotide
tcgtcgttttgtcgttttgtcgtt

SEQ ID NO: 60 - DNA, 20 bp, Artificial
  Synthetic oligonucleotide
tccatgacgttcctgatgct

SEQ ID NO: 61 - DNA, 43 bp, Artificial
  Synthetic oligonucleotide
agcgatgcgttcgagcatcgcutggaatttagggtagtattgt

SEQ ID NO: 62 - DNA, 18 bp, Artificial
  Synthetic oligonucleotide
ccctccaccaacatcaaa

SEQ ID NO: 63 - DNA, 21 bp, Artificial
  Synthetic oligonucleotide
tggaatttagggtagtattgt

SEQ ID NO: 64 - protein, 9 aa, Artificial
  Synthetic peptide
FLWGPRALV

SEQ ID NO: 65 - protein, 9 aa, Artificial
  Synthetic peptide
EVDPIGHLY

SEQ ID NO: 66 - protein, 10 aa, Artificial
  Synthetic peptide
MEVDPIGHLY

SEQ ID NO: 67 - protein, 10 aa, Artificial
  Synthetic peptide
VHFLLLKYRA

SEQ ID NO: 68 - protein, 10 aa, Artificial
  Synthetic peptide
LVHFLLLKYR

SEQ ID NO: 69 - protein, 10 aa, Artificial
  Synthetic peptide
LKYRAREPVT

SEQ ID NO: 70 - protein, 16 aa, Artificial
  Synthetic peptide
ACYEFLWGPRALVETS

SEQ ID NO: 71 - protein, 12 aa, Artificial
  Synthetic peptide
TQHFVQENYLEY

(19) United States
(12) Patent Application Publication
(10) Pub. No.: US 2012/0039993 A1
(43) Pub. Date: Feb. 16, 2012

(54) Detection of Gene Expression

(75) Inventors: Gaetan Otto, Sart-Tilman (Liege) (BE); Catherine Bovie, Sart-Tilman (Liege) (BE)

(73) Assignees: GLAXOSMITHKLINE BIOLOGICALS SA, Rixensart (BE); MDXHEALTH SA, Sart-Tilman (Liege) (BE)

(21) Appl. No.: 13/256,865

(22) PCT Filed: Mar. 17, 2010
(86) PCT No.: PCT/EP10/01674

§ 371 Date: Oct. 27, 2011

Related U.S. Application Data
(60) Provisional application No. 61/160,935, filed on Mar. 17, 2009.

(51) Int. Cl. A61K 9/127; A61P 35/00; A61K 39/385; C07K 14/435; C07H 21/04; C12Q 1/68

(52) U.S. Cl. 424/450; 536/24.3; 536/24.33; 435/6.11; 424/277.1; 530/350; 424/192.1; 536/23.1

(57) ABSTRACT

An oligonucleotide, primer or probe comprises the nucleotide sequence of any of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 61, 62 or 63. The oligonucleotide, primer or probe is useful for the detection of the methylation status of a gene. The oligonucleotides find application in the diagnosis and treatment of cancer.

### FIELD OF THE INVENTION

The present invention is concerned with the detection of CTAG1B, CTAG2, PRAME and/or MageA3 gene expression. More specifically, the invention relates to methods of detecting methylated or unmethylated forms of CTAG1B, CTAG2, PRAME and/or MageA3 and associated oligonucleotides, primers, probes, primer pairs and kits. The methods of the invention involve amplification techniques, in particular fluorescence based real-time and end-point PCR methods, and have diagnostic, prognostic and therapeutic utility.

### BACKGROUND TO THE INVENTION

### Cancer Testis (CT) Antigens

Cancer testis (CT) antigens are a class of tumour-associated antigens with expression normally restricted to germ cells in the testis, ovaries or trophoblast cells. These antigens are not usually expressed in adult somatic tissues (Simpson, et al., Nat. Rev. Cancer, 5(8):615-625 (2005); Scanlan, et al., Immunol. Reviews, 188:22-32 (2002); Scanlan, et al., Cane. Immun., 4:1-15 (2004)).

The gene regulation of CT antigens is disrupted in cancer patients, leading to the aberrant expression of these antigens in a wide variety of tumours. The first CT antigen to be identified, MAGE-1, was identified in the early 1990s by T-cell epitope cloning (van der Bruggen et al, 1991 Science 13; 254(5038):1643-7; van der Bruggen et al, 1999 Science 254:1643-1647; Traversah, et al, 1992 Immunogenetics, 35(3):145-152; and U.S. Pat. No. 5,342,774, incorporated by reference). Since then, serological expression cloning technique (SEREX) (Sahin, et al., Proc. Natl. Acad. Sci. USA, 92(25):11810-11813 (1995) and U.S. Pat. No. 5,698,396), recombinant antigen expression on yeast surface (RAYS) (Mischo, et al., Cane. Immun., 3:5-16 (2003)) and differential mRNA expression analysis (Gure, et al., Int. J. Cane, 85(5):726-732 (2000)) have led to the identification of more CT antigens. The immunogenicity of some CT antigens in cancer patients makes them an ideal target for the development of tumour vaccines.

The identification of tumour-associated antigens recognized by cellular or humoral effectors of the immune system has opened new perspectives for cancer immunotherapy. The concept of immunotherapy is based on the assumption that antigenic proteins expressed in tumors can be used as targets in therapeutic approaches employing the autologous immune system. Antigen-Specific Cancer Immunotherapeutics (ASCI) allow targeted treatment. ASCI have two principal components: “tumor antigens” to direct the immune response specifically against the cancer cell and “adjuvant systems” that comprise immuno-stimulation compounds selected to increase the anti-tumour immune response.

CTAG1B. A cancer testis antigen currently of interest for use in cancer immunotherapy is cancer/testis antigen 1B encoded by the CTAG1B gene (also known under gene alias NY-ESO-1). This antigen was first identified by SEREX in an oesophageal squamous cell carcinoma in the late 90's at the New York Branch of the Ludwig Institute for Cancer Research (Chen, et al., PNAS USA, 94(5):1914-1918 (1997); and U.S. Pat. No. 5,804,381, incorporated by reference). The protein CTAG1B is 180 amino acids in length and has been found in a wide variety of tumours, including but not limited to ovarian cancer, lung cancer, breast cancer, prostate cancer, oesophageal cancer, bladder cancer and in melanomas. (Konishi J et al. Oncol Rep. 2004 May; 11(5):1063-7; Nicholaou T et al, Immunol Cell Biol. 2006 June; 84(3):303-17; Sugita Y et al. Cancer Res. 2004 Mar. 15; 64(6):2199-204; Velazquez E F et al. Cancer Immun. 2007 Jul. 12; 7:11 and Jungbluth et al. 2001, Int. J. Cane, 92(6):856-860). Spontaneous humoral and cellular immune responses against this antigen have been described in patients with CTAG1B-positive tumours, and a number of HLA (Human Leukocyte Antigen) class I- and II-restricted peptides have been identified (Jager, et al., 1998 J. Exp. Med., 187(2):265-270; Yamaguchi, et al., 2004 Clin. Cane. Res., 10(3):890-961; and Davis, et al., 2004 Proc. Natl. Acad. Sci. USA, 101 (29):10697-10702). Exemplary of the patent literature are U.S. Pat. Nos. 6,140,050; 6,251,603; 6,242,052; 6,274,145; 6,338,947; 6,417,165; 6,525,177; 6,605,711; 6,689,742; 6,723,832; 6,756,044; and 6,800,730, all incorporated by reference.

In a clinical trial, three partially overlapping CTAG1B derived peptides with binding motifs to HLA-A2 (157-167, 157-165 and 155-163) have been used in a vaccine to treat twelve patients with metastatic NY-ESO-1 expressing tumours. This study demonstrated that synthetic NY-ESO-1 peptides can be administered safely and are capable of generating potentially beneficial T cell responses (Jager, et al., 2000 PNAS USA, 97(22):12198-12203).

A number of MHC (major histocompatibility complex) class I and II epitopes in the protein have been identified by different groups. The collagen-like region in the N-terminal contains at least one MHC class I epitope referred to herein as A31. The central region comprises several MHC class 2 epitopes referred to herein as DR1, DR2, DR4, DR7 and DP4. This region also contains several MHC class I epitopes referred to herein as B35, B51, Cw3 and Cw6. The C-terminal is believed to contain at least two class II epitopes (DR4 and DP4) and one class I epitope (A2).

CTAG2. Because of its high sequence similarity with CTAG1, a further cancer testis antigen of possible interest for immunotherapy is CTAG2 (also known as gene alias LAGE-1). Two CTAG2 transcripts have been described, LAGE-I a and LAGE-I b. LAGE-I b is incompletely spliced and codes for a putative protein of approximately 210 amino acids residues, while the LAGE-I a gene product contains 180 amino acid residues (Sun et al. Cancer Immunol Immunother 2006:55:644-652).

The N-terminal regions of the LAGE-1 and NY-ESO-1 proteins are highly conserved and are thought to have more than 97% identity. However, LAGE-1 differs from NY-ESO-1 in the central regions which are only 62% identical. The C-terminals of NY-ESO-1 and LAGE-Ia are highly conserved (more than 97% identity). However, the C-terminal of LAGE-I b is longer and is thought to have less than 50% identity with the same region in LAGE-1 a/NY-ESO-1.

General information relating to these two proteins is available from the LICR web site (see www.cancerimmunity.org/CTdatabase).

PRAME. Preferentially expressed antigen of melanoma, PRAME, was first isolated as a gene encoding a human melanoma antigen recognized by melanoma reactive cytotoxic T-cells (CTL). PRAME has not been designated a CT gene due to its trace level of expression in some normal adult tissues, including endometrium and adrenal glands. However, PRAME does exhibit the other main characteristics of CT genes, i.e. strong expression in the testis and up-regulation in various tumours including melanoma (97%), sarcoma (80%), small-cell lung cancer (70%), renal cell carcinoma (40%), and head and neck cancer (29%). Five alternatively spliced transcript variants encoding the same protein have been observed for this gene. The putative protein of 509 amino acids possesses well-characterized epitopes presented on HLA_A24 and HLA-A2 molecules.

MageA3. MAGE genes belong to the family of cancer/testis antigens. The MAGE family of genes comprises over 20 members and is made up of MAGE A, B, C and D genes (Scanlan et al, (2002) Immunol Rev. 188:22-32; Chomez et al, (2001) Cancer Res. 61(14):5544-51). They are clustered on chromosome X (Lucas et al., 1998 Cancer Res. 58.743-752; Lucas et al., 1999 Cancer Res 59:4100-4103; Lucas et al., 2000 Int J Cancer 87:55-60; Lurquin et al., 1997 Genomics 46:397-408; Muscatelli et al., 1995 Proc Natl Acad Sci USA 92:4987-4991; Pold et al., 1999 Genomics 59:161-167; Rogner et al 1995 Genomics 29:725-731), and have a yet undefined function (Ohman et al 2001 Exp Cell Res. 265(2): 185-94). The MAGE genes are highly homologous and the members of the MAGE-A family, especially, have between 60-98% homology.

The human MAGE-A3 gene is expressed in various types of tumours, including melanoma (Furuta et al. 2004 Cancer Sci. 95, 962-968.), bladder cancer, hepatocellular carcinoma (Qiu et al. 2006. Clinical Biochemistry 39, 259-266), gastric carcinoma (Honda et al. 2004 British Journal of Cancer 90, 838-843), colorectal cancer (Kim et al. 2006 World Journal of Gastroenterology 12, 5651-5657) and lung cancer (NSCLC) (Scanlan et al 2002 Immunol Rev. 188:22-32; Jang et al 2001 Cancer Res. 61, 21: 7959-7963). No expression has been observed in any normal adult tissues with the only exception of testicular germ cells or placenta (Haas et al. 1988 Am J Reprod Immunol Microbiol 18:47-51; Takahashi et al. 1995 Cancer Res 55:3478-382).

It is important to have quantitative high throughput assays capable of specifically identifying CTAG1B-, CTAG2- and/or PRAME-expressing patients that would benefit from immunotherapy, monitoring CTAG1B-, CTAG2- and/or PRAME-expression for dosage purpose, identifying CTAG1B-, CTAG2- and/or PRAME-expressing samples in clinical trials, or simply to identify at an early stage patients with cancer. A number of applicable diagnostic methods have been described and apply RNA extraction and RT-PCR (in for instance: Odunsi et al., Cancer Research 63, 6076-6083, 2003; Sharma et al., Cancer Immunity, Vol. 3, p. 19, 2003).

The greatest disadvantage of the existing assays is that they require RNA isolation to assess CTAG1B, CTAG2 and/or PRAME expression. Formalin-Fixed, Paraffin-Embedded (FFPE) tumour tissue is the usual method of tumour tissue preservation within clinical centres. The fixation in formalin changes the structure of molecules of RNA within the tissue, causing cross linking and also partial degradation. The partial degradation leads to the creation of smaller pieces of RNA of between 100-300 base pairs. These structural changes to the RNA limit the use of RNA extracted from FFPE tissue to measure CTAG1B-, CTAG2- and/or PRAME expression levels.

### Gene Methylation

Gene methylation is an important regulator of gene expression. In particular, methylation at cytosine residues found in CpG di-nucleotide pairs in the promoter region of specific genes can contribute to many disease conditions through down regulation of gene expression. For example, aberrant methylation of tumour suppressor genes can lead to up or down regulation of these genes and is thus associated with the presence and development of many cancers (Hoffmann et al. 2005 Biochem Cell Biol 83: 296-321). Patterns of aberrant gene methylation are often specific to the tissue of origin. Accordingly, detection of the methylation status of specific genes may be of prognostic and diagnostic utility and can be used to both determine the relative stage of a disease and also to predict response to certain types of therapy (Laird. 2003 Nat Rev Cancer 3: 253-266).

The methylation status of CTAG1B-, CTAG2- and/or PRAME has been studied to some extent in cancer tissues. It has been suggested that DNA methylation is the molecular mechanism directly responsible for the high expression of PRAME gene in chronic myeloid leukemia (CLM). Analysis of the PRAME promoter, exon1 and exon2 has revealed that PRAME possesses three CpG islands: a 201 bp island located within the promoter, a 204 bp island within exon1 and a 310 bp island within exon2, of which only CpG islands within exon2 showed epigenetic regulation (Gomez-Roman et al. Leukemia Research 31 (2007) 1521-1528). In combination with DNA-truncation/transfection experiments with respect to DNA methylation, it was also shown that changes in the methylation pattern in defined parts of the regulatory regions of PRAME are sufficient for its upregulation in cells usually not expressing the gene.

The methylation status of LAGE-1 has also been studied (De Smet et al. (1999) Molecular and Cellular Biology: 7327-7335). Due to the high sequence similarity of LAGE-1 and LAGE-2/NY-ESO-1 it has been difficult to distinguish between the methylation-status of the promoter sequences of those two genes.

Methylation-Specific PCR (MSP) with visualization of the results on a gel (gel-based MSP assay) is widely used to determine epigenetic silencing of genes (Esteller M et al. Cancer Res 2001; 61:3225-9.), although quantitative tests using other technologies have been developed (Laird P W., Nat Rev Cancer 2003; 3:253-66; Eads et al. Nucleic Acids Res 2000; 28:E32; Mikeska T, et al. J Mol Diagn 2007).

A number of fluorescence based technologies are available for real-time monitoring of nucleic acid amplification reactions. One such technology is described in U.S. Pat. No. 6,090,552 and EP 0912597 and is commercially known as Amplifluor®. This method is also suitable for end-point monitoring of nucleic acid amplification reactions. Vlassenbroeck et al. (Vlassenbroeck et al., 2008. Journal of Molec. Diagn., V10, No. 4) describe a standardized direct, real-time MSP assay with use of the Amplifluor® technology.

An object of the present invention is to provide improved assays that eliminate the disadvantages of the existing assays.

### SUMMARY OF THE INVENTION

The present invention relates to improved methods and/or assays of measuring expression. The present invention further relates to certain types of therapy, in particular Antigen-Specific Cancer Immunotherapeutics (ASCI) based treatment of patients identified as expressing CTAG1B, CTAG2, PRAME and/or MageA3 through use of oligonucleotides, primers, probes, primer pairs, kits and/or methods as described herein. CTAG1B, CTAG2, PRAME and/or MageA3 (protein) expression is detected by determining the methylation status of the CTAG1B, CTAG2, PRAME and/or MageA3 gene rather than measuring the expression level of the gene itself. The inventors show that the methylation status result of their methylation tests is in good concordance with results obtained with the RT-PCR assay for CTAG1B, CTAG2 and/or PRAME expression in NSCLC, melanoma and breast cancer samples. In case of samples, e.g. fine needle biopsies from non-Small Lung Cancer, for which qRT-PCR prove difficult, protein expression detected by determining the methylation status of the MageA3 gene provides a valuable alternative. The assays are thus useful for selecting patients (suitable) for treatment, for predicting the likelihood of successful treatment of a patient and can be used to aid patient therapy selection.

In one aspect, the present invention provides an oligonucleotide, primer or probe comprising or consisting essentially of or consisting of the nucleotide sequence of any of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 61, 62 or 63; which oligonucleotide, primer or probe is useful for the detection of the methylation status of a gene.

The oligonucleotide, primer or probe preferably comprises, consists essentially of or consists of the following contiguous sequences in 5′ to 3′ order:
(a) a first nucleotide sequence of between approximately 6 and 30 nucleotides, wherein a nucleotide within said first nucleotide sequence is labelled with a first moiety selected from the donor moiety and the acceptor moiety of a molecular energy transfer pair, wherein the donor moiety emits fluorescence at one or more particular wavelengths when excited, and the acceptor moiety absorbs and/or quenches said fluorescence emitted by said donor moiety;
(b) a second, single-stranded nucleotide sequence comprising, consisting essentially of or consisting of between approximately 3 and 20 nucleotides;
(c) a third nucleotide sequence comprising, consisting essentially of or consisting of between approximately 6 and 30 nucleotides, wherein a nucleotide within said third nucleotide sequence is labelled with a second moiety selected from said donor moiety and said acceptor moiety, and said second moiety is the member of said group not labelling said first nucleotide sequence, wherein said third nucleotide sequence is complementary in reverse order to said first nucleotide sequence such that a duplex can form between said first nucleotide sequence and said third nucleotide sequence such that said first moiety and second moiety are in proximity such that, when the donor moiety is excited and emits fluorescence, the acceptor moiety absorbs and quenches said fluorescence emitted by said donor moiety; and
(d) at the 3′ end of the primer, a fourth, single-stranded nucleotide sequence comprising, consisting essentially of or consisting of between approximately 8 and 40 nucleotides that comprises at its 3′ end the nucleotide sequence of any of SEQ ID NO. 2, 4, 6, 8, 10, 12, 14, 16, 18, 20, 22, 24, 26, 28, 30, 32, 34, 36, 38, 40, 42, 44, 62 and 63;
   wherein when said duplex is not formed, said first moiety and said second moiety are separated by a distance that prevents molecular energy transfer between said first and second moiety.

The specific nucleotide sequences at the 3′ end permit the methylation status of the CTAG1B, CTAG2, PRAME and/or MageA3 gene to be determined. These primers bind preferentially to unmethylated forms of the CTAG1B, CTAG2, PRAME and/or MageA3 gene following treatment with an appropriate reagent (as discussed herein). Properties of these oligonucleotides are discussed herein, which discussion applies mutatis mutandis. The specific nucleotide sequences are able to prime synthesis by a nucleic acid polymerase of a nucleotide sequence complementary to a nucleic acid strand comprising the portion of the methylated or unmethylated DNA of the CTAG1B, CTAG2, PRAME and/or MageA3 family gene.

Most preferably, the oligonucleotide, primer or probe consists of the nucleotide sequence of SEQ ID NO. 1, 3, 5, 7, 9, 11, 13, 15, 17, 19, 21, 23, 25, 27, 29, 31, 33, 35, 37, 39, 41 or 61 and is used to amplify a portion of the gene of interest.

Further provided is a primer pair comprising a primer comprising or consisting essentially of or consisting of the nucleotide sequence of any of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 61, 62 or 63. Suitable primer pairs may be readily determined by one skilled in the art, based on a sense and an antisense primer directing amplification of an appropriate portion of the relevant gene (as discussed herein). Examples of primer pairs of the invention are set forth in Table 1. Claim 6 also recites suitable primer pairs.

In a further aspect, there is provided a kit comprising at least one primer, primer pair or set of primers comprising or consisting essentially of or consisting of the nucleotide sequence of any of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 61, 62 or 63. The kit is for detecting the methylation status of a gene, in particular a gene such as CTAG1B, CTAG2, PRAME and/or MageA3.

In a further aspect, the invention provides for a method of detecting the methylation status of the CTAG1B, CTAG2, PRAME and/or MageA3 gene in a DNA-containing sample, comprising:
(a) contacting/treating the DNA-containing sample with a reagent which selectively modifies unmethylated cytosine residues in the DNA to produce detectable modified residues but which does not modify methylated cytosine residues
(b) amplifying at least a portion of the methylated or unmethylated gene of interest using at least one primer pair, at least one primer of which is designed to bind only to the sequence of methylated or unmethylated DNA respectively following treatment with the reagent, wherein at least one primer in the primer pair comprises, consists essentially of, or consists of a nucleotide sequence of any of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 61, 62 or 63 (as appropriate).

In a further aspect, there is provided a method of diagnosing cancer or predisposition to cancer comprising detecting the methylation status of the CTAG1B, CTAG2, PRAME and/or MageA3 gene in a sample by using an oligonucleotide, primer or probe, primer pair, kit or a method as described herein, wherein the presence of unmethylated CTAG1B, CTAG2, PRAME and/or MageA3 in the sample is indicative for cancer or predisposition to cancer.

In a further aspect, there is provided a method for determining the presence of CTAG1B, CTAG2, PRAME and/or MageA3 positive tumor comprising detecting the methylation status of the CTAG1B, CTAG2, PRAME and/or MageA3 gene in a sample by using an oligonucleotide, primer or probe, primer pair, kit or a method as described herein, wherein the presence of unmethylated CTAG1B, CTAG2, PRAME and/or MageA3 is indicative for the presence of a CTAG1B, CTAG2, PRAME and/or MageA3 positive tumor. By “CTAG1B, CTAG2, PRAME and/or MageA3 positive tumor” is meant any tumor or tumor cells (isolated from a patient) which express the CTAG1B, CTAG2, PRAME and/or MageA3 antigen.

The invention further provides a method for identifying and/or selecting a patient suitable for treatment with a CTAG1B, CTAG2, PRAME and/or MageA3 immunotherapeutic comprising detecting the methylation status of the CTAG1B, CTAG2, PRAME and/or MageA3 gene in a sample of the patient by using an oligonucleotide, primer or probe, primer pair, kit or a method as described herein, wherein if the CTAG1B, CTAG2, PRAME and/or MageA3 gene is unmethylated the subject is (preferably) identified and/or selected for treatment with the CTAG1B, CTAG2, PRAME and/or MageA3 immunotherapeutic. Thus, patients with unmethylated CTAG1B, CTAG2, PRAME and/or MageA3 are preferred to those in which the gene is methylated.

Alternatively, if the gene is not unmethylated the subject is preferably not identified and/or selected for treatment with a CTAG1B, CTAG2, PRAME and/or MageA3 immunotherapeutic.

In a related aspect, the invention provides a method for predicting the likelihood of successful treatment of cancer comprising detecting the methylation status of the CTAG1B, CTAG2, PRAME and/or MageA3 gene in a sample of the patient by using an oligonucleotide, primer or probe, primer pair, kit or a method as described herein, wherein if the gene is unmethylated the likelihood of successful treatment with a C CTAG1B, CTAG2, PRAME and/or MageA3 immunotherapeutic is higher than if the gene is methylated.

Alternatively, the absence of unmethylated CTAG1B, CTAG2, PRAME and/or MageA3 in the sample indicates that the likelihood of resistance to treatment with a CTAG1B, CTAG2, PRAME and/or MageA3 immunotherapeutic is higher than if the gene is unmethylated. Thus, the detection of a methylated CTAG1B, CTAG2, PRAME and/or MageA3 gene indicates that the probability of successful treatment with an immunotherapeutic is low.

In a further related aspect, the invention provides a method of selecting a suitable treatment regimen for cancer comprising detecting the methylation status of the CTAG1B, CTAG2, PRAME and/or MageA3 gene in a sample of the patient by using an oligonucleotide, primer or probe, primer pair, kit or a method as described herein, wherein if the gene is unmethylated, an immunotherapeutic is selected for treatment.

Alternatively, if the gene is not unmethylated, treatment with an immunotherapeutic is contra-indicated.

Also provided is a method of treating cancer in a subject comprising administration of an immunotherapeutic, wherein the subject has been selected for treatment on the basis of measuring the methylation status of a CTAG1B, CTAG2, PRAME and/or MageA3 gene, according to any of the methods of the invention or by using an oligonucleotide, primer or probe, primer pair, kit or a method as described herein.

Preferably, for all of the different aspects described herein, the detection of unmethylated CTAG1B, CTAG2, PRAME and/or MageA3 gene corresponds to an increased level of CTAG1B, CTAG2, PRAME and/or MageA3 protein.

The present invention further provides a method of treating a patient comprising: measuring the methylation status of a CTAG1B, CTAG2, PRAME and/or MageA3 gene according to any of the methods of the invention and/or by using an oligonucleotide, primer or probe, primer pair, kit or a method as described herein, and then administering to the patient a composition comprising CTAG1B, CTAG2, PRAME and/or MageA3 as described herein. The composition is preferably administered if the CTAG1B, CTAG2, PRAME and/or MageA3 gene is found to be unmethylated.

In a further aspect there is provided a method of treating a patient susceptible to recurrence of a CTAG1B, CTAG2, PRAME and/or MageA3 expressing tumour, the patient having been treated to remove tumour tissue, the method comprising: measuring the methylation status of a CTAG1B, CTAG2, PRAME and/or MageA3 gene in the tumour tissue, according to any of the methods of the invention or by using an oligonucleotide, primer or probe, primer pair, kit or a method as described herein, and then administering to the patient a composition comprising CTAG1B, CTAG2, PRAME and/or MageA3 as described herein. The composition is preferably administered if the CTAG1B, CTAG2, PRAME and/or MageA3 gene is found to be unmethylated.

In a still further aspect of the present invention there is provided a use of a composition comprising CTAG1B, CTAG2, PRAME and/or MageA3 in the manufacture of a medicament for the treatment of a patient suffering from a tumour, in which a patient has been selected for treatment on the basis of measuring the methylation status of a CTAG1B, CTAG2, PRAME and/or MageA3 gene, according to any of the methods of the invention or by using an oligonucleotide, primer or probe, primer pair, kit or a method as described herein. There is also provided a composition comprising CTAG1B, CTAG2, PRAME and/or MageA3 for use in the treatment of a patient suffering from a tumour, in which a patient has been selected for treatment on the basis of measuring the methylation status of a CTAG1B, CTAG2, PRAME and/or MageA3 gene, according to any of the methods of the invention or by using an oligonucleotide, primer or probe, primer pair, kit or a method as described herein.

In a yet further embodiment there is provided a use of a composition comprising CTAG1B, CTAG2, PRAME and/or MageA3 in the manufacture of a medicament for the treatment of a patient susceptible to recurrence of a CTAG1B, CTAG2, PRAME and/or MageA3 expressing tumour, in which a patient has been selected for treatment on the basis of measuring the methylation status of a CTAG1B, CTAG2, PRAME and/or MageA3 gene, according to any of the methods of the invention or by using an oligonucleotide, primer or probe, primer pair, kit or a method as described herein. There is also provided a composition comprising CTAG1B, CTAG2, PRAME and/or MageA3 for use in the treatment of a patient susceptible to recurrence of a CTAG1B, CTAG2, PRAME and/or MageA3 expressing tumour, in which a patient has been selected for treatment on the basis of measuring the methylation status of a CTAG1B, CTAG2, PRAME and/or MageA3 gene, according to any of the methods of the invention or by using an oligonucleotide, primer or probe, primer pair, kit or a method as described herein.

### DETAILED DESCRIPTION OF THE INVENTION

The invention provides an assay for detecting the presence and/or amount of a methylated or unmethylated CTAG1B, CTAG2, PRAME and/or MageA3 gene in a DNA-containing sample. To develop this assay, it was necessary to identify regions susceptible to methylation in the CTAG1B, CTAG2, PRAME and/or MageA3 gene and to develop particular oligonucleotides that could differentiate unmethylated from methylated forms of the CTAG1B, CTAG2, PRAME and/or MageA3 gene.

Accordingly, in a first aspect, the present invention provides an oligonucleotide, primer or probe comprising or consisting essentially of or consisting of the nucleotide sequence of any of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 61, 62 or 63. These oligonucleotides are useful in the detection of the methylation status of a gene of interest. The oligonucleotides may serve as primers and/or probes. In certain embodiments, the oligonucleotides detect the unmethylated form of the gene. In certain embodiments, these oligonucleotides comprise a hairpin structure as described herein and represented by SEQ ID NO. 43. Such preferred oligonucleotides comprise, consist essentially of or consist of the nucleotide sequences according to SEQ ID NO; 1, 3, 5, 7, 9, 11, 13, 15, 17, 19, 21, 23, 25, 27, 29, 31, 33, 35, 37, 39, 41 or 61 for detecting the unmethylated form of the gene.

The “genes” or “gene of interest” of the invention are preferably CTAG1B and/or CTAG2 and/or PRAME and/or MageA3 genes.

“CTAG1B” is the gene symbol approved by the HUGO Gene Nomenclature Committee. The gene is located on chromosome X; (location: Xq28) and the gene sequence is listed under the accession numbers U87459 and NM 001327. The ensemble gene ID is ENSG00000184033. The gene encodes the cancer/testis antigen 1B [Homo sapiens]. CTAG1B is often referred to interchangeably as “CTAG1B” OR “CTAG” OR “CTAG1” OR “NY-ESO-1” OR “ESO1” OR “LAGE-2” OR “LAGE2B”, all are used herein. Hypomethylation of this gene may be linked to the incidence of cancers, such as melanoma, lung cancer (including NSCLC), prostate cancer or ovarian cancer for example.

“CTAG2” is the gene symbol approved by the HUGO Gene Nomenclature Committee. The gene is located on chromosome X; (location: Xq28) and the gene sequence is listed under the accession number AJ012833. The ensemble gene ID is ENSG00000126890. The gene encodes the cancer/testis antigen 2 [Homo sapiens]. CTAG2 is often referred to interchangeably as “CTAG2” OR “CAMEL” OR “ESO2” LAGE-1 OR “LAGE-2b” OR “MGC3803” OR “MGC138724”, all are used herein. Hypomethylation of this gene may be linked to the incidence of cancers, such as melanoma, lung cancer (including NSCLC), bladder cancer, prostate cancer, head and neck cancer, ovarian cancer, cervical cancer, colorectal cancer, esophageal carcinoma or hepatocellular carcinoma. “PRAME” is the gene symbol approved by the HUGO Gene Nomenclature Committee. The gene is located on Chromosome 22 (location: 22q11.22) and the gene sequence is listed under the accession numbers U65011 and NM 206953. The ensemble gene ID is ENSG00000185686. The gene encodes the preferentially expressed antigen in melanoma [Homo sapiens]. PRAME is often referred to interchangeably as “MAPE” or “OIP4”; all are used herein. Hypomethylation of this gene may be linked to the incidence of cancers, such as cervical cancer, prostate cancer, lung cancer, ovarian cancer, breast cancer or head and neck squamous cell carcinoma.

MAGEA3 and MAGEA6 are the gene symbols approved by the HUGO Gene Nomenclature Committee. The MAGEA3 gene is located on chromosome X (location q28) and the gene sequence is listed under the accession numbers NM_{—}005362 and ENSG00000197172. The MAGE-A3 gene encodes melanoma antigen family A, 3. The MAGEA6 gene is located on chromosome X (location q28). MAGE-A3 is often referred to interchangeably as MAGE-3 or MAGEA3. Likewise, MAGE-A6 is often referred to interchangeably as MAGE-6 or MAGEA6; all are used herein. Hypomethylation of these genes may be linked to the incidence of cancers, such as melanoma or lung cancer (including NSCLC) for example.

CpG dinucleotides susceptible to methylation are typically concentrated in the promoter and/or exon and/or intron regions of human genes. In certain embodiments, the methylation status of the gene is assessed by determining levels of methylation in the promoter, intron, exon1 and/or exon2 region of the gene. A “promoter” is a region upstream from the transcription start site (TSS), extending between approximately 10 Kb, 4 Kb, 3 Kb, 1 Kb, 500 bp or 150 to 300 bp from the TSS. When the CpG distribution in the promoter region is rather scarce, levels of methylation may be assessed in the intron and/or exon regions. The region for assessment may be a region that comprises both intron and exon sequences and thus overlaps both regions.

The term “methylation state” or “methylation status” refers to the presence or absence of 5-methylcytosine (“5-mCyt”) at one or a plurality of CpG dinucleotides within a DNA sequence. “Hypermethylation” is defined as an increase in the level of methylation above normal levels. Thus, it relates to aberrant methylation of cytosine (5-mCyt) at specific CpG sites in a gene, often in the promoter region. Normal levels of methylation may be defined by determining the level of methylation in non-cancerous cells for example.

“Hypomethylation” refers to a decreased presence of 5-mCyt at one or a plurality of CpG dinucleotides within a DNA sequence (of a test DNA sample), relative to the amount of 5-mCyt found at corresponding CpG dinucleotides within a “normal” DNA sequence (found in a suitable control sample). Again, “normal” levels of methylation may be defined by determining the level of methylation in non-cancerous cells for example. In this invention, hypomethylation of the CTAG1B, CTAG2 and/or PRAME gene (or MageA3 in certain aspects) is indicative of an increased expression of this tumour associated antigen gene which provides a reliable indicator of cancer.

The invention provides in a second aspect a method of detecting the presence and/or amount of a methylated or unmethylated gene in a DNA-containing sample comprising the step of contacting the DNA-containing sample with at least one oligonucleotide comprising, consisting essentially of, or consisting of the nucleotide sequence of any SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 61, 62 or 63. The method preferably comprises the further step of assessing whether the gene is methylated or unmethylated. This may depend upon whether the oligonucleotide stably binds to the DNA in the DNA-containing sample, as discussed herein.

Techniques for assessing methylation status are based on distinct approaches. Any suitable technique, applying the oligonucleotides of the invention may be employed. In one embodiment, approaches for detecting methylated CpG dinucleotide motifs use a reagent which selectively modifies unmethylated cytosine residues in the DNA to produce detectable modified residues. The reagent does not modify methylated cytosine residues and thus allows for discrimination between unmethylated and methylated nucleic acid molecules in a downstream process, which preferably may involve nucleic acid amplification. The reagent may, in one embodiment, act to selectively deaminate unmethylated cytosine residues. Thus, following exposure to the reagent the unmethylated DNA contains a different nucleotide sequence to that of corresponding methylated DNA. The deamination of cytosine results in a uracil residue being present, which has the same base pairing properties as thymine, and thus differs from cytosine's base pairing behaviour. This makes the discrimination between methylated and non-methylated cytosines possible.

Useful conventional techniques for assessing sequence differences use oligonucleotide primers. Two approaches to primer design are possible. Firstly, primers may be designed that themselves do not cover any potential sites of DNA methylation. Sequence variations at sites of differential methylation are located between the two primer binding sites and visualisation of the sequence variation requires further assay steps. Secondly, primers may be designed that hybridize specifically with either the methylated or unmethylated version of the initial treated sequence. After hybridization, an amplification reaction can be performed and amplification products assayed using any detection system known in the art. The presence of an amplification product indicates that the primer hybridized to the DNA. The specificity of the primer indicates whether the DNA had been modified or not, which in turn indicates whether the DNA had been methylated or not. If there is a sufficient region of complementarity, e.g., 12, 13, 14, 15, 16, 17, 18, 19, 20, 21 or 22 nucleotides, to the target, then the primer may also contain additional nucleotide residues that do not interfere with hybridization but may be useful for other manipulations. Examples of such other residues may be sites for restriction endonuclease cleavage, for ligand binding or for factor binding or linkers or repeats, or residues for purpose of visualization. The oligonucleotide primers may or may not be such that they are specific for modified methylated residues. Preferred oligonucleotides for use as primers comprise, consist essentially of, or consist of the nucleotide sequence of any of SEQ ID NO. 1, 3, 5, 7, 9, 11, 13, 15, 17, 19, 21, 23, 25, 27, 29, 31, 33, 35, 37, 39, 41 or 61.

A further and often complementary way to distinguish between modified and unmodified nucleic acid is to use oligonucleotide probes. Such probes may hybridize directly to modified nucleic acid or to further products of modified nucleic acid, such as products obtained by amplification. Probe-based assays exploit the oligonucleotide hybridisation to specific sequences and subsequent detection of the hybrid. There may also be further purification steps before the amplification product is detected e.g. a precipitation step. Oligonucleotide probes may be labeled using any detection system known in the art. These include but are not limited to fluorescent moieties, radioisotope labelled moieties, bioluminescent moieties, luminescent moieties, chemiluminescent moieties, enzymes, substrates, receptors, or ligands. Oligonucleotides for use as probes may comprise, consist essentially of, or consist of the nucleotide sequence of any of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 61, 62 or 63. The preferred oligonucleotide probe preferably comprises, consists essentially of or consists of the nucleotide sequence selected from SEQ ID No. 2, 4, 6, 8, 10, 12, 14, 16, 18, 20, 22, 24, 26, 28, 30, 32, 34, 36, 38, 40, 42, 44 and 61.

“Oligonucleotide primer” is referred to herein interchangeably as “primer”. Likewise, “oligonucleotide probe” is referred to herein interchangeably as “probe”.

In preferred embodiments, the methylation status of the gene (or portion thereof, especially the CpG islands) is determined using methylation specific PCR (MSP).

The MSP technique will be familiar to one of skill in the art. In the MSP approach, DNA may be amplified using primer pairs designed to distinguish unmethylated from methylated DNA by taking advantage of sequence differences as a result of sodium-bisulfite treatment (Herman J G et al. Proc Natl Acad Sci USA. 1996 Sep. 3; 93(18):9821-6 and WO 97/46705). A specific example of the MSP technique is designated real-time quantitative MSP (QMSP), which permits reliable quantification of methylated DNA in real time.

Real-time methods are generally based on the continuous optical monitoring of an amplification procedure and utilise fluorescently labelled reagents whose incorporation in a product can be quantified and whose quantification is indicative of copy number of that sequence in the template. Such labeled reagent may be a fluorescent dye that preferentially binds double-stranded DNA and whose fluorescence is greatly enhanced by binding of double-stranded DNA. Alternatively, labeled primers and/or labeled probes can be used. They represent a specific application of the well known and commercially available real-time amplification techniques such as TAQMAN®, MOLECULAR BEACONS®, AMPLIFLUOR® and SCORPION® DzyNA®, etc. Often, these real-time methods are used with the polymerase chain reaction (PCR).

TaqMan technology uses linear, hydrolytic oligonucleotide probes that contain a fluorescent dye and a quenching dye. When irradiated, the excited fluorescent dye transfers energy to the nearby quenching dye molecule rather than fluoresencing (FRET principle). TaqMan probes anneal to an internal region of the PCR product and are cleaved by the exonuclease activity of the polymerase when it replicates a template. This ends the activity of the quencher, and the reporter dye starts to emit fluorescence which increases in each cycle proportional to the rate of probe cleavage.

Molecular beacons also contain fluorescent and quenching dyes, but they are designed to adopt a hairpin structure while free in solution to bring both dyes in close proximity for Fluorescence Resonance Energy Transfer (FRET) to occur. When the beacon hybridises to the target during the annealing step, the hairpin linearises and both dyes (donor and acceptor/quencher) are separated. The increase in fluorescence detected from the donor will correlate to the amount of PCR product available.

With scorpion probes, sequence-specific priming and PCR product detection is achieved using a single oligonucleotide. The scorpion probe maintains a stem-loop configuration in the unhybridized state and FRET occurs between the fluorophore and quencher. The 3′ portion of the stem also contains a sequence that is complementary to the extension product of the primer. This sequence is linked to the 5′ end of a specific primer via a non-amplifiable monomer. After extension of the scorpion primer, the specific probe sequence is able to bind to its complement within the extended amplicon, thus opening up the hairpin loop, separating the fluorophore and quencher, and providing a fluorescence signal.

In Heavymethyl, the priming is methylation specific, but non-extendable oligonucleotide blockers provide this specificity instead of the primers themselves. The blockers bind to bisulfite-treated DNA in a methylation-specific manner, and their binding sites overlap the primer binding sites. When the blocker is bound, the primer cannot bind and therefore the amplicon is not generated. Heavymethyl can be used in combination with real-time detection.

The Plexor™ qPCR and qRT-PCR Systems take advantage of the specific interaction between two modified nucleotides to achieve quantitative PCR analysis. One of the PCR primers contains a fluorescent label adjacent to an iso-dC residue at the 5′ terminus. The second PCR primer is unlabeled. The reaction mix includes deoxynucleotides and iso-dGTP modified with the quencher dabcyl. Dabcyl-iso-dGTP is preferentially incorporated at the position complementary to the iso-dC residue. The incorporation of the dabcyl-iso-dGTP at this position results in quenching of the fluorescent dye on the complementary strand and a reduction in fluorescence, which allows quantitation during amplification. For these multiplex reactions, a primer pair with a different fluorophore is used for each target sequence.

Thus the oligonucleotides comprising, consisting essentially of, or consisting of the nucleotide sequence of any of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 61, 62 or 63 may be employed as primers or probes in the aforementioned methods for detection of the methylation status of a gene of interest.

In a preferred embodiment, the invention provides a real-time method of detecting the presence and/or amount of a methylated or unmethylated gene of interest in a DNA-containing sample, comprising:

(a) contacting/treating the DNA-containing sample with a reagent which selectively modifies unmethylated cytosine residues in the DNA to produce detectable modified residues but which does not modify methylated cytosine residues
(b) amplifying at least a portion of the methylated or unmethylated gene of interest using at least one primer pair, at least one primer of which is designed to bind only to the sequence of methylated or unmethylated DNA respectively following treatment with the reagent, wherein at least one primer in the primer pair comprises, consists essentially of, or consists of the nucleotide sequence of any of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 61, 62 or 63.

The gene of interest in the methods of the invention is preferably the CTAG1B, CTAG2, MageA3 and/or PRAME gene. Preferably, at least one primer in the primer pair is a primer containing a stem loop structure carrying a donor and an acceptor moiety of a molecular energy transfer pair arranged such that in the absence of amplification, the acceptor moiety quenches fluorescence emitted by the donor moiety (upon excitation) and during amplification, the stem loop structure is disrupted so as to separate the donor and acceptor moieties sufficiently to produce a detectable fluorescence signal. This may be detected in real-time to provide an indication of the presence of the methylated or unmethylated gene of interest. The primer in the primer pair which comprises, consists essentially of, or consists of the nucleotide sequence of any of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 61, 62 or 63 preferably carries the stem loop structure.

In certain embodiments the gene copy number of the methylated or unmethylated gene is determined. Here, the method described herein preferably comprises a further step: (c) quantifying the results of the real-time detection against a standard curve for the methylated or unmethylated gene of interest to produce an output of gene copy number.

Preferably, step (c) is further characterised in that the amplification is considered valid where the cycle threshold value is less or equal than 40.

For genes such as the CTAG1B, CTAG2, MageA3 and/or PRAME gene, detection of an unmethylated version of the gene may be of primary relevance.

The methods of the invention allow the presence of a methylated or unmethylated gene of interest to be detected in a sample in real-time. Since the methods of the invention are quantitative methods, the (relative) amounts of the methylated or unmethylated form of the gene of interest can also be determined as the reaction proceeds.

Real-time methods do not need to be utilised, however. Analyses can be performed only to discover whether the target DNA is present in the sample or not. End-point amplification detection techniques utilize the same approaches as widely used for Real Time PCR. Therefore, the methods of the invention may encompass an end-point method of detecting the presence and/or amount of a methylated or unmethylated gene of interest in a DNA-containing sample.

Thus, the invention provides an (end point) method of detecting the presence and/or amount of a methylated or unmethylated gene of interest in a DNA-containing sample, comprising:

(a) contacting and/or treating the DNA-containing sample with a reagent which selectively modifies unmethylated cytosine residues in the DNA to produce detectable modified residues but which does not modify methylated cytosine residues
(b) amplifying at least a portion of the methylated or unmethylated gene of interest using at least one primer pair, at least one primer of which is designed to bind only to the sequence of methylated or unmethylated DNA respectively following treatment with the reagent, wherein at least one primer in the primer pair comprises, consists essentially of, or consists of the nucleotide sequence of any of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 61, 62 or 63.

As aforementioned, the gene of interest in the methods of the invention is preferably the CTAG1B, CTAG2, MageA3 and/or PRAME. Preferably, at least one primer in the primer pair is a primer containing a stem loop structure carrying a donor and an acceptor moiety of a molecular energy transfer pair having the characteristics as described herein. The primer in the primer pair which comprises, consists essentially of, or consists of the nucleotide sequence of any of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 61, 62 or 63 preferably carries the stem loop structure.

For the CTAG1B, CTAG2, MageA3 and/or PRAME genes detection of an unmethylated version of the gene may be of primary relevance. Primers comprising, consisting essentially of, or consisting of the nucleotide sequence of any of SEQ ID NO. 1, 3, 5, 7, 9, 11, 13, 15, 17, 19, 21, 23, 25, 27, 29, 31, 33, 35, 37, 39, 41 or 61 have been designed for the purpose of detecting unmethylated CTAG1B, CTAG2 and/or PRAME DNA following treatment with the reagent.

The absence of unmethylated gene is indicative of the presence of methylated gene.

In case a gene copy number of the methylated or unmethylated gene is desired, the method may comprise a further step:

(c) quantifying the results of the detection against a standard curve for the methylated or unmethylated gene of interest to produce an output of gene copy number.

All embodiments of the invention are applicable to the end-point aspects of the invention and thus apply mutatis mutandis. End point analysis may invoke use of a fluorescent plate reader or other suitable instrumentation to determine the fluorescence at the end of the amplification.

The methods of the invention are most preferably ex vivo or in vitro methods carried out on any suitable (DNA containing) test sample. In one embodiment, however, the method may also include the step of obtaining the sample. The test sample is a DNA-containing sample, in particular a DNA-containing sample including the gene of interest. The methods of the invention can be used in the diagnosis of disease, in particular where methylation of a gene of interest is (known to be) linked to the incidence of disease.

The DNA-containing sample may comprise any suitable tissue sample or body fluid. Preferably, the test sample is obtained from a human subject. For cancer applications, the sample may comprise a tissue sample taken from the tissue suspected of being cancerous or from a representative bodily fluid.

Hypomethylation of the CTAG1B, CTAG2, MageA3 and/or PRAME gene has been associated with lung cancer and thus the invention may be applied to lung cancer. Thus, in one embodiment, the test sample to be used in the methods of the invention involving a CTAG1B, CTAG2, MageA3 and/or PRAME gene preferably contains lung cells or nucleic acid (molecules) from lung cells. Most preferably, the sample is a Formalin Fixed Paraffin Embedded (FFPE) tissue. There are two types of lung cancer: non-small cell lung cancer (NSCLC) and small cell lung cancer (SCLC). The names simply describe the type of cell found in the tumours. The test sample preferably contains cells or nucleic acid from non-small cell lung carcinoma (NSCLC). NSCLC includes squamous-cell carcinoma, adenocarcinoma, and large-cell carcinoma and accounts for around 80% of lung cancers. In a preferred embodiment, where the cancer is a NSCLC, the sample is a lung tissue sample or a sputum sample or a serum sample. NSCLC is difficult to cure and treatments available tend to have the aim of prolonging life as far as possible and relieving symptoms of disease. NSCLC is the most common type of lung cancer and is associated with poor outcomes.

Hypomethylation of the CTAG1B, CTAG2, MageA3 and/or PRAME gene is also linked to melanoma and thus the invention may be applied to melanoma. Melanoma is a pigmented, readily accessible lesion that has been well defined in histopathological terms. Early radial growth phase (RGP) melanomas can invade into the epidermis and papillary dermis, but have no capacity for metastasis; resection at this stage is almost completely curative. A subsequent vertical growth phase (VGP) denotes a transition to a more aggressive stage, which is capable of metastasis. Changes in gene expression occurring at the RGP/VGP transition are, thus, of great interest. Thus, in additional embodiments, a further preferred test sample to be used in the methods of the invention contains melanoma cells or nucleic acid from melanoma cells. Preferably, the test sample is obtained from a skin lesion.

Hypomethylation of the CTAG1B, CTAG2, MageA3 and/or PRAME gene is also linked to breast cancer and thus the invention may be applied to breast cancer. There are two major groups of breast cancer: noninvasive carcinoma and invasive carcinoma. The noninvasive carcinomas include lobular carcinoma in situ and ductal carcinoma in situ. Unfortunately, breast cancers often grow through the basement membrane and roughly 95% of all breast cancers are infiltrating or invasive carcinomas. The most common type of invasive breast cancer (about 75%) is invasive ductal carcinoma, arising in the milk ducts and spreading through the duct walls. Invasive lobular carcinoma originates in the milk glands and accounts for 10 to 15% of invasive breast cancers. Less common types of invasive breast cancer include the following: inflammatory breast cancer, Paget's disease of the nipple, medullary carcinoma, mucinous carcinoma, phyllodes tumor, and tubular carcinoma. Rarely (about 1%), sarcomas (cancer of the connective tissue) develop in the breasts. Individuals may develop one, the other, or a combination of invasive and noninvasive breast cancer. Thus, in additional embodiments, a further preferred test sample to be used in the methods of the invention contains breast cells or nucleic acid molecules from breast cells. Most preferably, the test sample is obtained from a breast tissue.

Other DNA-containing samples for use in the methods of the invention include samples for diagnostic, prognostic, or personalised medicinal uses. These samples may be obtained from surgical samples, such as biopsies or fine needle aspirates, from paraffin embedded tissues, from frozen tumor tissue samples, from fresh tumour tissue samples or from a fresh or frozen body fluid, for example. Non-limiting examples include whole blood, bone marrow, cerebrospinal fluid, peritoneal fluid, pleural fluid, lymph fluid, serum, plasma, urine, chyle, stool, ejaculate, sputum, nipple aspirate, saliva, swabs specimens, colon wash specimens and brush specimens. The tissues and body fluids can be collected using any suitable method, many such methods are well known in the art. Assessment of a paraffin-embedded specimen can be performed directly or on a tissue section.

The terms “sample”, “patient sample” and “sample of the patient” are used interchangeably and are intended to mean a DNA-containing sample from a patient, as described above.

The methods of the invention may be carried out on purified or unpurified DNA-containing samples. However, in a preferred embodiment, prior to step (a) (the reagent treatment step) or as a preliminary step, DNA is isolated/extracted/purified from the DNA-containing sample. Any suitable DNA isolation technique may be utilised. Examples of purification techniques may be found in standard texts such as Molecular Cloning—A Laboratory Manual (Third Edition), Sambrook and Russell (see in particular Appendix 8 and Chapter 5 therein). In one preferred embodiment, purification involves alcohol precipitation of DNA. Preferred alcohols include ethanol and isopropanol. Suitable purification techniques also include salt-based precipitation methods. Thus, in one specific embodiment the DNA purification technique comprises use of a high concentration of salt to precipitate contaminants. The salt may comprise, consist essentially of or consist of potassium acetate and/or ammonium acetate for example. The method may further include steps of removal of contaminants which have been precipitated, followed by recovery of DNA through alcohol precipitation.

In an alternative embodiment, the DNA purification technique is based upon use of organic solvents to extract contaminants from cell lysates. Thus, in one embodiment, the method comprises use of phenol, chloroform and isoamyl alcohol to extract the DNA. Suitable conditions are employed to ensure that the contaminants are separated into the organic phase and that DNA remains in the aqueous phase.

Further kits use magnetic beads, silica-membrane, etc. Such kits are well known in the art and commercially available. The methods of the invention may use the PUREGENE® DNA Purification Kit.

In preferred embodiments of these purification techniques, extracted DNA is recovered through alcohol precipitation, such as ethanol or isopropanol precipitation.

Formalin-Fixed, Paraffin-Embedded (FFPE) tumour tissue is the usual method of tumour tissue preservation within clinical centres. Such FFPE embedded samples require a dewaxing step prior to DNA extraction. In a preferred embodiment, FFPE tissue samples or sample material immobilized on slides are first dewaxed by xylene treatment. The contact period with the xylene should be sufficient to allow the xylene to contact and interact with the sample. In a more preferred embodiment, FFPE samples are deparaffinized in 100% xylene for about 2 hours. This step may be repeated once more to ensure complete deparaffinization. After xylene treatment samples are rehydrated using 70% ethanol.

The methods of the invention may also, as appropriate, incorporate (also prior to step (a) or as a preliminary step) quantification of isolated/extracted/purified DNA in the sample. Quantification of the DNA in the sample may be achieved using any suitable means. Quantitation of nucleic acids may, for example, be based upon use of a spectrophotometer, a fluorometer or a UV transilluminator. Examples of suitable techniques are described in standard texts such as Molecular Cloning—A Laboratory Manual (Third Edition), Sambrook and Russell (see in particular Appendix 8 therein). In a preferred embodiment, kits such as the Picogreen® dsDNA quantitation kit available from Molecular Probes, Invitrogen may be employed to quantify the DNA.

The methods of the invention may rely upon a reagent which selectively modifies unmethylated cytosine residues in the DNA to produce detectable modified residues. The mode of action of the reagent has been explained already. In a preferred embodiment, the reagent which selectively modifies unmethylated cytosine residues in the DNA to produce detectable modified residues but which does not modify methylated cytosine residues comprises, consists essentially of or consists of a bisulphite reagent (Frommer et al., Proc. Natl. Acad. Sci. USA 1992 89:1827-1831,). Several bisulphite containing reagents are known in the art and suitable kits for carrying out the deamination reaction are commercially available (such as the EZ DNA methylation kit from Zymo Research). A particularly preferred reagent for use in the methods of the invention comprises, consists essentially of or consists of sodium bisulphite.

Once the DNA in the sample has been treated with the reagent, it is then necessary to detect the difference in nucleotide sequence caused by the reagent. This is done using a nucleic acid amplification technique. As mentioned already, functionally relevant methylation is most commonly associated with the promoter regions. In particular, so called “CpG islands” include a relatively high incidence of CpG residues and are often found near the Transcription start site of the gene. For some genes such as for example MAGE-3, the CpG distribution in the promoter region is rather scarce. In such case it may be appropriate to assess intron and exon regions of genes for methylation. Various software programs exist to allow CpG islands in a gene of interest to be identified. Accordingly, the methods of the invention may involve amplifying at least a portion of the methylated or unmethylated gene of interest using at least one primer pair. As discussed above, since the residues of interest whose methylation status is to be investigated, are typically found in defined CpG islands and/or in the promoter region and/or intron regions and/or exon regions of the gene of interest, the primer pair will typically amplify only a portion of the gene (in this region), rather than the entirety. Any suitable portion of the gene may be amplified according to the methods of the invention, provided that the amplification product is detectable as a reliable indicator of the presence of the gene of interest. Particularly readily detectable amplification products are between approximately 50 and 250 bp. Even more preferably, amplification using the at least one primer pair for amplification of the methylated or unmethylated gene of interest produces an amplification product of between approximately 100 and 200 bp or between 50 and 100 bp. This is particularly relevant for tissue samples, especially paraffin embedded samples where limited DNA quality is typically obtained and smaller amplicons may be desired. In a preferred embodiment, the detectable amplification product comprises at least the nucleotide sequence of any of SEQ ID NO. 2, 4, 6, 8, 10, 12, 14, 16, 18, 20, 22, 24, 26, 28, 30, 32, 34, 36, 38, 40, 42, 44, 62 and 63. Preferably, an amplification product of (around) 50 bp, 51 bp, 52 bp, 53 bp, 54 bp, 55 bp, 56 bp, 57 bp, 58 bp, 59 bp, 60 bp, 61 bp, 62 bp, 63 bp, 64 bp, 65 bp, 66 bp, 67 bp, 68 bp, 69 bp, 70 bp, 71 bp, 72 bp, 73 bp, 74 bp, 75 bp, 76 bp, 77 bp, 78 bp, 79 bp, 80 bp, 81 bp, 82 bp, 83 bp, 84 bp, 85 bp, 86 bp, 87 bp, 88 bp, 89 bp, 90 bp, 91 bp, 92 bp, 93 bp, 94 bp, 95 bp, 96 bp, 97 bp, 98 bp, 99 bp, 100 bp, 101 bp, 102 bp, 103 bp, 104 bp, 105 bp, 106 bp, 107 bp, 108 bp, 109 bp, 110 bp, 111 bp, 112 bp, 113 bp, 114 bp, 115 bp, 116 bp, 117 bp, 118 bp, 119 bp, 120 bp, 121 bp, 122 bp, 123 bp, 124 bp, 125 bp, 126 bp, 127 bp, 128 bp, 129 bp, 130 bp, 131 bp, 132 bp, 133 bp, 134 bp, 135 bp, 136 bp, 137 bp, 138 bp, 139 bp, 140 bp, 141 bp, 142 bp 143 bp, 144 bp, 145 bp, 146 bp, 147 bp, 148 bp, 149 bp, or 150 bp is produced.

At least one primer in the primer pair, and preferably both primers, is designed to bind only to the sequence of methylated or unmethylated DNA following treatment with the reagent. Thus, the primer acts to discriminate between a methylated and an unmethylated gene by base pairing only with either the methylated form of the gene (which remains unmodified following treatment with the reagent) or the unmethylated form of the gene (which is modified by the reagent) depending upon the application to which the methods are put. The primer must, therefore, cover at least one methylation site in the gene of interest. Preferably, the primer binds to a region of the gene including at least 1, 2, 3, 4, 5, 6, 7 or 8 methylation sites. Most preferably the primer is designed to bind to a sequence in which all cytosine residues in CpG pairs within the primer binding site are methylated or unmethylated—i.e. a “fully methylated” or a “fully unmethylated” sequence. However, if only a single or a few methylation sites are of functional relevance, the primer may be designed to bind to a target sequence in which only these residues must be methylated (remain as a cytosine) or unmethylated (converted to uracil) for effective binding to take place. Other (non-functionally relevant) potential sites of methylation may be avoided entirely through appropriate primer design or primers may be designed which bind independently of the methylation status of these less relevant sites (for example by including a mix of G and A residues at the appropriate location within the primer sequence). Accordingly, an amplification product is expected only if the methylated or unmethylated form of the gene of interest was present in the original DNA-containing sample. Additionally or alternatively, it may be appropriate for at least one primer in the primer pair to bind only to the sequence of unmethylated DNA following treatment with the reagent and the other primer to bind to methylated DNA only following treatment—for example where a gene involves functionally important sites which are methylated and separate functionally important sites which are unmethylated.

Preferably, at least one primer in the primer pair is a primer containing a stem loop or “hairpin” structure carrying a donor and an acceptor moiety of a molecular energy transfer pair. This primer may or may not be a primer which discriminates between methylated and unmethylated DNA as desired. The primer is arranged such that in the absence of amplification, the acceptor moiety quenches fluorescence emitted by the donor moiety upon excitation. Thus, prior to, or in the absence of, amplification directed by the primer the stem loop or “hairpin” structure remains intact. Fluorescence emitted by the donor moiety is effectively accepted by the acceptor moiety leading to quenching of fluorescence.

During amplification, the configuration of the stem loop or hairpin structure of the primer is altered. In particular, once the primer is incorporated into an amplification product, and in particular into a double stranded DNA, (particularly during the second round of amplification) the stem loop or hairpin structure is disrupted. This alteration in structure separates the donor and acceptor moieties sufficiently that the acceptor moiety is no longer capable of effectively quenching the fluorescence emitted by the donor moiety. Thus, the donor moiety produces a detectable fluorescence signal. This signal is detected in real-time to provide an indication of the gene copy number of the methylated or unmethylated gene of interest.

Thus, the methods of the invention may utilise oligonucleotides for amplification of nucleic acids that are detectably labelled with molecular energy transfer (MET) labels. The primers contain a donor and/or acceptor moiety of a MET pair and are incorporated into the amplified product of an amplification reaction, such that the amplified product contains both a donor and acceptor moiety of a MET pair.

When the amplified product is double stranded, the MET pair incorporated into the amplified product may be on the same strand or, when the amplification is triamplification, on opposite strands. In certain instances wherein the polymerase used in amplification has 5′-3′ exonuclease activity, one of the MET pair moieties may be cleaved from at least some of the population of amplified product by this exonuclease activity. Such exonuclease activity is not detrimental to the amplification methods of the invention.

The methods of the invention, as discussed herein are adaptable to many methods for amplification of nucleic acid sequences, including polymerase chain reaction (PCR), triamplification, and other amplification systems.

In a preferred embodiment, the MET is fluorescence resonance energy transfer (FRET), in which the oligonucleotides are labelled with donor and acceptor moieties, wherein the donor moiety is a fluorophore and the acceptor moiety may be a fluorophore, such that fluorescent energy emitted by the donor moiety is absorbed by the acceptor moiety. The acceptor moiety may be a quencher. Thus, the amplification primer is a hairpin primer that contains both donor and acceptor moieties, and is configured such that the acceptor moiety quenches the fluorescence of the donor. When the primer is incorporated into the amplification product its configuration changes, quenching is eliminated, and the fluorescence of the donor moiety may be detected.

The methods of the invention permit detection of an amplification product without prior separation of unincorporated oligonucleotides. Moreover, they allow detection of the amplification product directly, by incorporating the labelled oligonucleotide into the product.

In a preferred embodiment, the methods of the invention also involve determining the expression of a reference gene. Reference genes are important to allow comparisons to be made between different samples. By selecting an appropriate gene believed to be expressed in a stable and reliable fashion between the samples to be compared, detecting amplification of a reference gene together with the gene of interest takes into account inter-sample variability, such as amount of input material, enzymatic efficiency, sample degradation etc. A reference gene should ideally, in the presence of a reliable amount of input DNA, be one which is constantly expressed between the samples under test. Thus, the results from the gene of interest can be normalised against the corresponding copy number of the reference gene. Suitable reference genes for the present invention include beta-actin, glyceraldehyde-3-phosphate dehydrogenase (GAPDH), ribosomal RNA genes such as 18S ribosomal RNA and RNA polymerase II gene (Radonic A. et al., Biochem Biophys Res Commun. 2004 Jan. 23; 313(4):856-62). In a particularly preferred embodiment, the reference gene is beta-actin.

Thus the methods of the invention may be further characterised in amplifying at least a portion of a reference gene using at least one primer pair, wherein at least one primer in the primer pair is a primer containing a stem loop structure having the aforementioned characteristics.

Any suitable portion of the reference gene may be amplified according to the methods of the invention, provided that the amplification product is detectable as a reliable indicator of the presence of the reference gene. Particularly readily detectable amplification products are between approximately 50 and 250 bp. Even more preferably, amplification using the at least one primer pair for amplification of the reference gene produces an amplification product of between approximately 50 bp and 150 bp. This is particularly relevant for tissue samples, especially paraffin embedded samples where limited DNA quality is typically obtained.

In the embodiments in which a reference gene is included in the methods of the invention the methods may be further characterised in that the step of the methods which comprises quantifying the results of the (real-time) detection against a standard curve for the methylated or unmethylated gene of interest also comprises quantifying the results of the real-time detection of the reference gene against a standard curve for the reference gene to produce an output of gene copy number in each case and optionally further comprises normalising the results by dividing the gene copy number of the methylated or unmethylated gene of interest by the gene copy number of the reference gene.

Again, the methods may be characterised in that the amplification is considered valid where the cycle threshold value is less or equal than 40.

Amplification of at least a portion of the reference gene generally utilises at least one primer pair. Preferably, at least one primer in the primer pair is a primer containing a stem loop structure carrying a donor and an acceptor moiety of a molecular energy transfer pair, as for the gene of interest. The mode of action of such structure during amplification has been explained herein.

The “hairpin” primers for use in the methods of the invention are most preferably as described in U.S. Pat. No. 6,090,552 and EP 0912597, the disclosures of which are hereby incorporated in their entirety. These primers are commercially known as Amplifluor® primers. Thus, in a particularly preferred embodiment, the primer containing a stem loop structure used to amplify a portion of the gene of interest and/or reference gene comprises, consists essentially of or consists of the following contiguous sequences in 5′ to 3′ order:

(a) a first nucleotide sequence of between approximately 6 and 30 nucleotides, wherein a nucleotide within said first nucleotide sequence is labelled with a first moiety selected from the donor moiety and the acceptor moiety of a molecular energy transfer pair, wherein the donor moiety emits fluorescence at one or more particular wavelengths when excited, and the acceptor moiety absorbs and/or quenches said fluorescence emitted by said donor moiety;
(b) a second, single-stranded nucleotide sequence comprising, consisting essentially of or consisting of between approximately 3 and 20 nucleotides;
(c) a third nucleotide sequence comprising, consisting essentially of or consisting of between approximately 6 and 30 nucleotides, wherein a nucleotide within said third nucleotide sequence is labelled with a second moiety selected from said donor moiety and said acceptor moiety, and said second moiety is the member of said group not labelling said first nucleotide sequence, wherein said third nucleotide sequence is complementary in reverse order to said first nucleotide sequence such that a duplex can form between said first nucleotide sequence and said third nucleotide sequence such that said first moiety and second moiety are in proximity such that, when the donor moiety is excited and emits fluorescence, the acceptor moiety absorbs and quenches said fluorescence emitted by said donor moiety; and
(d) at the 3′ end of the primer, a fourth, single-stranded nucleotide sequence comprising, consisting essentially of or consisting of between approximately 8 and 40 nucleotides that comprises at its 3′ end a sequence of any of SEQ ID NO. 2, 4, 6, 8, 10, 12, 14, 16, 18, 20, 22, 24, 26, 28, 30, 32, 34, 36, 38, 40, 42, 44, 62 and 63 (and thus is able to prime synthesis by a nucleic acid polymerase of a nucleotide sequence complementary to a nucleic acid strand comprising the portion of the methylated or unmethylated DNA of the gene); wherein when said duplex is not formed, said first moiety and said second moiety are separated by a distance that prevents molecular energy transfer between said first and second moiety.

In a particularly preferred embodiment, the donor moiety and acceptor moiety form a fluorescence resonance energy transfer (FRET) pair. Molecular energy transfer (MET) is a process by which energy is passed non-radiatively between a donor molecule and an acceptor molecule. Fluorescence resonance energy transfer (FRET) is a form of MET. FRET arises from the properties of certain chemical compounds; when excited by exposure to particular wavelengths of light, they emit light (i.e., they fluoresce) at a different wavelength. Such compounds are termed fluorophores. In FRET, energy is passed non-radiatively over a long distance (10-100 Å) between a donor molecule, which is a fluorophore, and an acceptor molecule. The donor absorbs a photon and transfers this energy nonradiatively to the acceptor (Förster, 1949, Z. Naturforsch. A4: 321-327; Clegg, 1992, Methods Enzymol. 211: 353-388). When two fluorophores whose excitation and emission spectra overlap are in close proximity, excitation of one fluorophore will cause it to emit light at wavelengths that are absorbed by and that stimulate the second fluorophore, causing it in turn to fluoresce. In other words, the excited-state energy of the first (donor) fluorophore is transferred by a resonance induced dipole—dipole interaction to the neighbouring second (acceptor) fluorophore. As a result, the lifetime of the donor molecule is decreased and its fluorescence is quenched, while the fluorescence intensity of the acceptor molecule is enhanced and depolarized. When the excited-state energy of the donor is transferred to a non-fluorophore acceptor, the fluorescence of the donor is quenched without subsequent emission of fluorescence by the acceptor. In this case, the acceptor functions as a quencher. Both quenchers and acceptors may be utilised in the present invention. Pairs of molecules that can engage in fluorescence resonance energy transfer (FRET) are termed FRET pairs. In order for energy transfer to occur, the donor and acceptor molecules must typically be in close proximity (up to 70 to 100 Å) (Clegg, 1992, Methods Enzymol. 211: 353-388; Selvin, 1995, Methods Enzymol. 246: 300-334). The efficiency of energy transfer falls off rapidly with the distance between the donor and acceptor molecules. According to Förster (1949, Z. Naturforsch. A4:321-327), the efficiency of energy transfer is proportional to D−10⁻⁶, where D is the distance between the donor and acceptor. Effectively, this means that FRET can most efficiently occur up to distances of about 70 Å. Molecules that are commonly used in FRET are discussed in a separate section. Whether a fluorophore is a donor or an acceptor is defined by its excitation and emission spectra, and the fluorophore with which it is paired. For example, FAM is most efficiently excited by light with a wavelength of 488 nm, and emits light with a spectrum of 500 to 650 nm, and an emission maximum of 525 nm. FAM is a suitable donor fluorophore for use with JOE, TAMRA, and ROX (all of which have their excitation maximum at 514 nm).

In one particularly preferred embodiment, said donor moiety is fluorescein or a derivative thereof, and said acceptor moiety is DABCYL. Preferably, the fluorescein derivative comprises, consists essentially of or consists of 6-carboxy fluorescein.

The MET labels can be attached at any suitable point in the primers. In a particularly preferred embodiment, the donor and acceptor moieties are positioned on complementary nucleotides within the stem loop structure, such that whilst the stem loop is intact, the moieties are in close physical proximity to one another. However, the primers of the invention may be labelled with the moieties in any position effective to allow MET/FRET between the respective donor and acceptor in the absence of amplification and separation of the donor and acceptor once the primer is incorporated into an amplification product.

The stem loop or hairpin structure sequence does not depend upon the nucleotide sequence of the target gene (gene of interest or reference gene) since it does not bind thereto. Accordingly, “universal” stem loop or hairpin sequences may be designed which can then be combined with a sequence specific primer to facilitate real-time detection of a sequence of interest. The main sequence requirement is that the sequence forms a stem loop/hairpin structure which is stable in the absence of amplification (and thus ensures efficient quenching). Thus, the sequence specific portion of the primer binds to a template strand and directs synthesis of the complementary strand. The primer therefore becomes part of the amplification product in the first round of amplification. When the complimentary strand is synthesised, amplification occurs through the stem loop/hairpin structure. This separates the fluorophore and quencher molecules, thus leading to generation of florescence as amplification proceeds.

The stem loop structure is preferably found at the 5′ end of the sequence specific portion of the primer used in the amplification.

As mentioned above, this detector sequence is generally labelled with a FRET pair. Preferably, one moiety in the FRET pair is found towards, near or at the 5′ end of the sequence and the other moiety is found towards, near or at the 3′ end of the sequence such that, when the stem loop or hairpin structure remains intact FRET is effective between the two moieties.

As detailed in the experimental section, primers must be carefully selected in order to ensure sensitivity and specificity of the methods of the invention. Accordingly, particularly preferred primers for use in detecting methylation status of the gene include a primer comprising, consisting essentially of or consisting of the nucleotide sequence set forth as:

(SEQ ID NO: 1)
5′- AGCGATGCGTTCGAGCATCGCUGGAAGGTGGGGGAGAGTG -3′
(SEQ ID NO. 2)
5′- AAAACAACACAACCCCAAAAA -3′
and/or,
(SEQ ID NO. 3)
5′- AGCGATGCGTTCGAGCATCGCUAAAACAACACAACCCCAAAAA -
3′
and/or,
(SEQ ID NO. 4)
5′- GGAAGGTGGGGGAGAGTG -3′
and/or,
(SEQ ID NO. 5)
5′- AGCGATGCGTTCGAGCATCGCUGGGTTGGAGAGTTGTTTGTTTG -
3′
and/or,
(SEQ ID NO. 6)
5′- CACATCTCCCCCACCTCCT -3′
and/or,
(SEQ ID NO. 7)
5′- AGCGATGCGTTCGAGCATCGCUCACATCTCCCCCACCTCCT -3′
and/or,
(SEQ ID NO. 8)
5′- GGGTTGGAGAGTTGTTTGTTTG -3′
and/or,
(SEQ ID NO. 9)
5′- AGCGATGCGTTCGAGCATCGCUTGGTGGTGTTGTTTTTGTGT -3′
and/or,
(SEQ ID NO. 10)
5′- CTTAACCCTATTATCTCCATCTC -3′
and/or,
(SEQ ID NO. 11)
5′- AGCGATGCGTTCGAGCATCGCUCTTAACCCTATTATCTCCATCT
C -3′
and/or,
(SEQ ID NO. 12)
5′- TGGTGGTGTTGTTTTTGTGT -3′
and/or,
(SEQ ID NO. 13)
5′- AGCGATGCGTTCGAGCATCGCUGGTGGTTTTGAAGGATTTTATT
G -3′
and/or,
(SEQ ID NO. 14)
5′- ACCCAACACCTTCCCTATCCT -3′
and/or,
(SEQ ID NO. 15)
5′- AGCGATGCGTTCGAGCATCGCUACCCAACACCTTCCCTATCCT -
3′
and/or,
(SEQ ID NO. 16)
5′- GGTGGTTTTGAAGGATTTTATTG -3′
and/or,
(SEQ ID NO. 17)
5′- AGCGATGCGTTCGAGCATCGCUTTTTGTTTTGGGATGTTGTATTT
T -3′
and/or,
(SEQ ID NO. 18)
5′- CCTCATCCACCCAACACCTT -3′
and/or,
(SEQ ID NO. 19)
5′- AGCGATGCGTTCGAGCATCGCUCCTCATCCACCCAACACCTT -3′
and/or,
(SEQ ID NO. 20)
5′-_TTTTGTTTTGGGATGTTGTATTTT -3′
and/or,
(SEQ ID NO. 21)
5′- AGCGATGCGTTCGAGCATCGCUTGGGTTTGTAGTGTTTTAGTATTG
TTT -3′
and/or,
(SEQ ID NO. 22)
5′-_TCCACCCTACTTTCCCTACATTC -3′
and/or,
(SEQ ID NO. 23)
5′- AGCGATGCGTTCGAGCATCGCUTCCACCCTACTTTCCCTACATT
C -3′
and/or,
(SEQ ID NO. 24)
5′-_TGGGTTTGTAGTGTTTTAGTATTGTTT -3′
and/or,
(SEQ ID NO. 25)
5′- AGCGATGCGTTCGAGCATCGCUTTGTTTTGGGATATTTTATTTGTT
TT -3′
and/or,
(SEQ ID NO. 26)
5′-_AAAAACTCCACCCTACTTTCC -3′
and/or,
(SEQ ID NO. 27)
5′- AGCGATGCGTTCGAGCATCGCUAAAAACTCCACCCTACTTTCC -
3′
and/or,
(SEQ ID NO. 28)
5′-_TTGTTTTGGGATATTTTATTTGTTTT -3′
and/or,
(SEQ ID NO. 29)
5′- AGCGATGCGTTCGAGCATCGCUGAGGGGAGGGGTGTGAATGTG -
3′
and/or,
(SEQ ID NO. 30)
5′-_CATTCCTCCCTACTCCCAAAAA -3′
and/or,
(SEQ ID NO. 31)
5′- AGCGATGCGTTCGAGCATCGCUCATTCCTCCCTACTCCCAAAAA -
3′
and/or,
(SEQ ID NO. 32)
5′-_GAGGGGAGGGGTGTGAATGTG -3′
and/or,
(SEQ ID NO. 33)
5′- AGCGATGCGTTCGAGCATCGCUTGGTGGATGTTTTGGGATTT -3′
and/or,
(SEQ ID NO. 34)
5′-_CAACATTTCTACCTCTACTCCCACCTT -3′
and/or,
(SEQ ID NO. 35)
5′- AGCGATGCGTTCGAGCATCGCUCAACATTTCTACCTCTACTCCCAC
CTT -3′
and/or,
(SEQ ID NO. 36)
5′-_TGGTGGATGTTTTGGGATTT -3′
and/or,
(SEQ ID NO. 37)
5′- AGCGATGCGTTCGAGCATCGCUGTTTTGGAAGGATTGAGAAATG
G -3′
and/or,
(SEQ ID NO. 38)
5′-_CACCCTAACCACTACATAAAACAAA -3′
and/or,
(SEQ ID NO. 39)
5′- AGCGATGCGTTCGAGCATCGCUCACCCTAACCACTACATAAAACAA
A -3′
and/or,
(SEQ ID NO. 40)
5′-_GTTTTGGAAGGATTGAGAAATGG -3′
and/or,
(SEQ ID NO. 41)
5′- AGCGATGCGTTCGAGCATCGCUTAGGGAGTATATAGGTTGGGGAAG
TT -3′
and/or,
(SEQ ID NO. 42)
5′-_AACACACAATAACAAACACAAATTCAC -3′
and/or,
(SEQ ID NO. 44)
5′- AGGGAGTATATAGGTTGGGGAAGTT -3′
and/or,
(SEQ ID NO. 61)
5′- AGCGATGCGTTCGAGCATCGCUTGGAATTTAGGGTAGTATTGT-
3′
and/or,
(SEQ ID NO. 62)
5′- CCCTCCACCAACATCAAA -3′
(SEQ ID NO. 63)
5′- TGGAATTTAGGGTAGTATTGT -3′

SEQ ID NO 4 and 8 represent forward primer (sense primer) sequences complementary to the bisulfite converted unmethylated sequence of CTAG1B.

SEQ ID NO 12, 16 and 20 represent forward primer (sense primer) sequences complementary to the bisulfite converted unmethylated sequence of CTAG2.

SEQ ID NO 24, 28, 32, 36 and 40 represent forward primer (sense primer) sequences complementary to the bisulfite converted unmethylated sequence of PRAME.

SEQ ID NO 43 represents the hairpin structure sequence

SEQ ID NO 1, 5, 9, 13, 17, 21, 25, 29, 33, and 37 comprise the hairpin structure sequence and the sequence of SEQ ID NO. 4, 8, 12, 16, 20, 24, 28, 32, 36 and 40 respectively.

SEQ ID NO. 2 and 6 represent the reverse primer (antisense primer) sequence complementary to the bisulfite converted unmethylated sequence of the CTAG1B promoter.

SEQ ID NO. 10, 14 and 18 represent the reverse primer sequence complementary to the bisulfite converted unmethylated sequence of the CTAG2 promoter.

SEQ ID NO. 22, 26, 30, 34 and 38 represent the reverse primer sequence complementary to the bisulfite converted unmethylated sequence of the PRAME promoter.

SEQ ID NO. 3, 7, 11, 15, 19, 23, 27, 31, 35 and 39 comprises the hairpin structure sequence and the sequence of SEQ ID NO. 2, 6, 10, 14, 18, 22, 26, 30, 34 and 38.

SEQ ID NO 42 and 44 represent the forward and the reverse primer sequence complementary to the bisulfite converted methylated sequence of the Actin Beta gene; SEQ ID NO. 41 comprise the hairpin structure sequence and the sequence of SEQ ID 44.

SEQ ID NO 63 represents the forward primer (sense primer) sequences complementary to the bisulfite converted unmethylated sequence of MageA3.

SEQ ID NO 61 comprises the hairpin structure sequence and the sequence of SEQ ID NO. 63.

SEQ ID NO. 62 represents the reverse primer sequence complementary to the bisulfite converted unmethylated sequence of MageA3.

As detailed in the experimental section, expression and methylation levels of CTAG2 showed best concordance for the assays that incorporated the primers of any of SEQ ID NO. 9 to 12 and SEQ ID NO 21 to 20. As detailed in the experimental section, expression and methylation levels of PRAME showed best concordance in the assays that incorporated the primers of any of SEQ ID NO. 21 to 40.

Thus in another embodiment, preferred primer binding to the a region of CTAG2 comprise, consist essentially of or consist of the nucleotide sequence of any of SEQ ID NO. 9 to 12 and SEQ ID NO 21 to 20. The preferred primer binding to a region of PRAME comprise, consist essentially of or consist of the nucleotide sequence of any of SEQ ID NO. 21 to 40.

Amplification products generated with the different hypomethylation assays had the following sequences:

CTAG1B_1 (129 bp)
(SEQ ID NO. 45)
GGAAGGTGGGGGAGAGTGGTTTGGATTTTAGTATTTTTTTTTTTTTTAGG
GTTAGGTTTTGTTTGGTTATTTTTTGTTGTTATAGGTGTGTTTGGTATAG
ATATTTAGTTTTTGGGGTTGTGTTGTTTT
CTAG1B_2 (130 bp)
(SEQ ID NO. 46)
GGGTTGGAGAGTTGTTTGTTTGAGTTGTATTTTGTTTTGTTTTGTTTTGT
TTTGATAGTTTTGGTGGTGAGGTGGGGGTTGGGAGATGGGGAGGGTAGGG
TTAGGTGGGGGAGGAGGTGGGGGAGATG
CTAG2 (150 bp)
(SEQ ID NO. 47)
TGGTGGTGTTGTTTTTGTGTAGGATGGAAGGTGTTTTTGTGGGGTTAGGA
GGTTGGATAGTTGTTTGTTTTAGTTGTATTTTGTTTTGTTTTGTTTTAGG
AGGTTTTGGTGGTGAGGTGGGGGTTGTGAGATGGAGATAATAGGGTTAAG
CTAG2_2 (80 bp)
(SEQ ID NO. 48)
GGTGGTTTTGAAGGATTTTATTGTGTTTGGTAATTTATTGTTTATGTTAG
TTTGGGATTAGGATAGGGAAGGTGTTGGGT
CTAG2_3 (125 bp)
(SEQ ID NO. 49)
TTTTGTTTTGGGATGTTGTATTTTTTTTTTGATTAGGGGTGGTTTTGAAG
GATTTTATTGTGTTTGGTAATTTATTGTTTATGTTAGTTTGGGATTAGGA
TAGGGAAGGTGTTGGGTGGATGAGG
PRAME_1: 129 bp
(SEQ ID NO. 50)
TGGGTTTGTAGTGTTTTAGTATTGTTTTGGGATATTTTATTTGTTTTTTA
GGTGTGATTTGTTAATAGGTTTGTATTGGTGATAAAAGGAGTAGTTTTGA
ATGTAGGGAAAGTAGGGTGGAGTTTTTTG
PRAME_2: 106 bp
(SEQ ID NO. 51)
TTGTTTTGGGATATTTTATTTGTTTTTTAGGTGTGATTTGTTAATAGGTT
TGTATTGGTGATAAAAGGAGTAGTTTTGAATGTAGGGAAAGTAGGGTGGA
GTTTTT
PRAME_3: 120 bp
(SEQ ID NO. 52)
GAGGGGAGGGGTGTGAATGTGTGGATTTTTGTGGAGAGTGGAAATATGGG
GAGTTGAGGGGAGTATGTGTGGGTTTTAGAAAGTTTTGGGAAATTGATTT
TTGGGAGTAGGGAGGAATG
PRAME_4: 59 bp
(SEQ ID NO. 53)
AGTGTTGGAGGTTTTGAGGTTAGTTTAAGTTGTTTTAAAATGGAATGAAG
GTGTTTGTG
PRAME _6: 50 bp
(SEQ ID NO. 54)
TGGTGGATGTTTTGGGATTTGGTTTTTTTGAAGGTGTTGGGGGTTGGGGA
TGGTTTAGGTAGTGGTGTAGGTGTTTTAGGAAGGTGGGAGTAGAGGTAGA
AATGTTG
PRAME_7: 98 bp
(SEQ ID NO. 55)
GTTTTGGAAGGATTGAGAAATGGGGATTGGTTAGATTAGGTTGTTTAGTT
TTTTGGTTTTTATTGTTGTTTTTTTTGTTTTATGTAGTGGTTAGGGTG

These sequences are located in CpG-rich islands of the respective genes. Accordingly, the invention further relates to oligonucleotides, primers and/or probes consisting of or complementary to parts of the bisulfite converted nucleotide sequences set forth as SEQ ID NO: 45, 46, 47, 48, 49, 50, 51, 52, 53, 54 or 55.

The portion of the primer consisting of or complementary to parts of the bisulfite converted sequence of the CTAG1B, CTAG2 and/or PRAME gene is preferably less than 30 bp; it is preferably 27, 26, 25, 24, 23, 22, 21, 20, 19, 18 or 17 bp in length. Thus the CTAG1B, CTAG2 and/or PRAME specific part of such preferred primer is preferably between 28 and 16 bp, or between 27 and 17 bp in length. The primer may thus comprise any sequence of 27, 26, 25, 24, 23, 22, 21, 20, 19, 18 or 17 consecutive bases consisting of or complementary to the bisulfite converted sequences.

Either one or both of the primers (in a primer pair) may be labelled with or synthesised to incorporate a suitable stem loop or hairpin structure carrying a donor and acceptor moiety, preferably at the 5′ end, as discussed in detail above. In a preferred embodiment, one or both of the primer(s) is labelled with or synthesised to incorporate, preferably at the 5′ end, the stem loop structure comprising, consisting essentially of or consisting of the nucleotide sequence set forth as

| | |
|---|---|
| 5′- AGCGATGCGTTCGAGCATCGCU -3′. | (SEQ ID NO: 43) |

This detector sequence is generally labelled with a FRET pair. Preferably, one moiety in the FRET pair is found towards, near or at the 5′ end of the sequence and the other moiety is found towards, near or at the 3′ end of the sequence such that, when the stem loop or hairpin structure remains intact FRET is effective between the two moieties. In a particularly preferred embodiment, the stem loop or hairpin structure, especially the nucleic acid comprising, consisting essentially of or consisting of the sequence set forth as SEQ ID NO: 1, is labelled at the 5′ end with FAM and at the 3′ end with DABCYL. Other preferred combinations are discussed herein, which discussion applies mutatis mutandis.

These primers form separate aspects of the present invention. Further characteristics of these primers are summarized in the detailed description (experimental part) below. It is noted that variants of the Oligonucleotides, primers and probes (sequences) may be utilised in the present invention. In particular, additional flanking sequences may be added, for example to improve binding specificity or the formation of a stem loop, as required. Variant sequences preferably have at least 90%, at least 91%, at least 92%, at least 93%, at least 94%, at least 95%, at least 96%, at least 97%, at least 98%, or at least 99% nucleotide sequence identity with the nucleotide sequences of the primers and/or probes set forth in SEQ ID NO:1 to 42 and SEQ ID NO: 44, 60, 61 and 62. The primers and hairpin structures may incorporate synthetic nucleotide analogues as appropriate or may be DNA, RNA or PNA based for example, or mixtures thereof. Similarly alternative fluorescent donor and acceptor moieties/FRET pairs may be utilised as appropriate. In addition to being labelled with the fluorescent donor and acceptor moieties, the primers may include modified oligonucleotides and other appending groups and labels provided that the functionality as a primer and/or stem loop/hairpin structure in the methods of the invention is not compromised.

For each primer pair at least one primer is labelled with a donor and an acceptor moiety of a molecular energy transfer pair arranged such that in the absence of amplification, the acceptor moiety quenches fluorescence emitted by the donor moiety (upon excitation) and during amplification, the stem loop structure is disrupted so as to separate the donor and acceptor moieties sufficiently to produce a detectable fluorescence signal which is detected in real-time to provide an indication of the gene copy number of the gene. Preferably, said donor moiety and said acceptor moiety are a FRET pair. In one embodiment, said donor moiety and said acceptor moiety are selected from 5-carboxyfluorescein or 6-carboxyfluorescein (FAM), 2′7′-dimethoxy-4′5′-dichloro-6-carboxyfluorescein (JOE), rhodamine, 6-carboxyrhodamine (R6G), N,N,N′-tetramethyl-6-carboxyrhodamine (TAMRA), 6-carboxy-X-rhodamine (ROX), 5-(2′-aminoethyl)aminonapthalene-1-sulfonic acid (EDANS), anthranilamide, coumarin, terbium chelate derivatives, Malachite green, Reactive Red 4, DABCYL, tetramethyl rhodamine, pyrene butyrate, eosine nitrotyrosine, ethidium, and Texas Red. In a further embodiment, said donor moiety is selected from fluorescein, 5-carboxyfluorescein or 6-carboxyfluorescein (FAM), rhodamine, 5-(2′-aminoethyl)aminonapthalene-1-sulfonic acid (EDANS), anthranilamide, coumarin, terbium chelate derivatives, Malachite green, and Reactive Red 4, and said acceptor moiety is selected from DABCYL, rhodamine, tetramethyl rhodamine, pyrene butyrate, eosine nitrotyrosine, ethidium, and Texas Red. Preferably, said donor moiety is fluorescein or a derivative thereof, and said acceptor moiety is DABCYL and most preferably the donor moiety is 6-carboxyfluorescein. Other preferred combinations, particularly in a multiplexing context, are discussed herein and these combinations are also envisaged for these aspects of the invention.

The invention also provides kits which may be used in order to carry out the methods of the invention. The kits may incorporate any of the preferred features mentioned in connection with the various methods (and uses) of the invention described herein. Thus, the invention provides a kit for detecting the presence and/or amount of a methylated or unmethylated gene of interest in a DNA-containing sample, comprising at least one primer pair of the invention. Preferably, the kit incorporates a primer pair of the invention for detecting the presence and/or amount of unmethylated and/or methylated CTAG1B, CTAG2, PRAME and/or MageA3 gene and a primer pair for detecting the presence and/or amount of a reference gene, in particular beta-actin. Thus, the kit may comprise primer pairs comprising a primer comprising, consisting essentially of or consisting of the nucleotide sequence set forth as SEQ ID NOs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 60, 61 or 62. Preferably, at least one primer in each primer pair is labelled with an appropriate stem loop or hairpin structure to facilitate detection in real-time, as discussed above (which discussion applies here mutatis mutandis). Most preferably at least one primer in each primer pair incorporates the stem loop or hairpin structure which comprises, consists essentially of or consists of the nucleotide sequence set forth as SEQ ID NO:43. The stem loop structure is labelled with an appropriate donor and acceptor moiety, as discussed herein (which discussion applies here mutatis mutandis).

As aforementioned, further characteristics of the primers of the invention are summarized in the detailed description (experimental part) below. Variants of these sequences may be utilised in the present invention as discussed herein. Alternative fluorescent donor and acceptor moieties/FRET pairs may be utilised as appropriate, as discussed herein.

In one embodiment, the kit of the invention further comprises a reagent which modifies unmethylated cytosine, as discussed herein (in preference to methylated cytosine resiudes which are protected). Such a reagent is useful for distinguishing methylated from unmethylated cytosine residues. In a preferred embodiment, the reagent comprises bisulphite, preferably sodium bisulphite. This reagent is capable of converting unmethylated cytosine residues to uracil, whereas methylated cytosines remain unconverted. This difference in residue may be utilised to distinguish between methylated and unmethylated nucleic acid in a downstream process, such as PCR using primers which distinguish between cytosine and uracil (cytosine pairs with guanine, whereas uracil pairs with adenine).

As discussed with respect to the methods of the invention herein, suitable controls may be utilised in order to act as quality control for the methods. Accordingly, in one embodiment, the kit of the invention further comprises, consists essentially of or consists of one or more control nucleic acid molecules of which the methylation status is known. These (one or more) control nucleic acid molecules may include both nucleic acids which are known to be, or treated so as to be, methylated and/or nucleic acid molecules which are known to be, or treated so as to be, unmethylated. One example of a suitable internal reference gene, which is generally unmethylated, but may be treated so as to be methylated, is beta-actin.

The kits of the invention may additionally include suitable buffers and other reagents for carrying out the claimed methods of the invention. Thus, the discussion provided in respect of the methods of the invention applies mutatis mutandis here and is not repeated for reasons of conciseness. In one embodiment, the kit of the invention further comprises, consists essentially of, or consists of nucleic acid amplification buffers.

The kit may also additionally comprise, consist essentially of or consist of enzymes to catalyze nucleic acid amplification. Thus, the kit may also additionally comprise, consist essentially of or consist of a suitable polymerase for nucleic acid amplification. Examples include those from both family A and family B type polymerases, such as Taq, Pfu, Vent etc.

The various components of the kit may be packaged separately in individual compartments or may, for example be stored together where appropriate.

The kit may also incorporate suitable instructions for use, which may be printed on a separate sheet or incorporated into the kit's packaging for example. The instructions may facilitate use of the kits of the invention with an appropriate real-time amplification or end-point detection apparatus, a number of which are commercially available.

The last step of the real-time methods of the invention involves quantifying the results of the real-time detection against a standard curve for the methylated or unmethylated gene of interest, and optionally the reference gene (where included). Standard curves may be generated using a set of standards. Each standard contains a known copy number, or concentration, of the gene of interest and/or reference gene as appropriate. Typically, a baseline value of fluorescence will be set to account for background fluorescence. For example, in one embodiment the Sequence Detection System (SDS) software is utilised. This software sets a default baseline range of cycles 3 to 15 of the amplification reaction before amplification products are detected. A threshold value of fluorescence is then defined at a statistically significant value above this baseline. Typically, the threshold is set to 10 standard deviations above the baseline fluorescence. Appropriate software is provided with apparatus for carrying out real-time amplification reactions. The software automatically calculates the baseline and threshold values for the reaction. The threshold cycle value (Ct) can then be determined for each standard. This is the number of cycles required to achieve the threshold amplification level. Thus, the greater the initial concentration of the gene standard in the reaction mixture, the fewer the number of cycles required to achieve a particular yield of amplified product. A plot of Ct against the log₁₀ of the known initial copy number of the set of standard DNAs produces a straight line. This is the standard curve. Thus, the Ct value for the amplification of the gene of interest and reference gene, where utilised, can each be interpolated against the respective standard curve in order to determine the copy number in the DNA-containing sample. Thus, the output of the method is the gene copy number for each of the gene of interest and reference gene. The results may be normalised by dividing the gene copy number of the methylated or unmethylated gene of interest by the gene copy number of the reference gene. In a preferred embodiment, the Applied Biosystems 7900 HT fast real-time PCR system is used to carry out the methods of the invention. Preferably, SDS software is utilised, preferably including a suitable algorithm such as the Auto CT algorithm for automatically generating baseline and threshold values for individual detectors.

Whilst the methods of the invention may be utilised with any suitable amplification technique, it is most preferred that amplification is carried out using the polymerase chain reaction (PCR). Thus, whilst PCR is a preferred amplification method, to include variants on the basic technique such as nested PCR, equivalents may also be included within the scope of the invention. Examples include, without limitation, isothermal amplification techniques such as NASBA, 3SR, TMA and triamplification, all of which are well known in the art and suitable reagents are commercially available. Other suitable amplification methods include, without limitation, the ligase chain reaction (LCR) (Barringer et al, 1990), MLPA, selective amplification of target polynucleotide sequences (U.S. Pat. No. 6,410,276), consensus sequence primed polymerase chain reaction (U.S. Pat. No. 4,437,975), invader technology (Third Wave Technologies, Madison, Wis.), strand displacement technology, arbitrarily primed polymerase chain reaction (WO90/06995) and nick displacement amplification (WO2004/067726).

The real-time PCR methods of the invention generally involve steps of lowering the temperature to allow primer annealing, raising the temperature for primer extension, raising the temperature for denaturation and lowering the temperature for data-collection. In one specific embodiment, the data-collection step is carried out at a temperature of between approximately 56° C. and 63° C., most preferably at approximately 57° C. or 62° C. since this has been shown to give maximally sensitive and specific results as discussed in the Examples section.

In a specific embodiment, the thermal profiling of the polymerase chain reaction comprises between 40 and 50 repeats, preferably approximately 45 repeats of the cycle:

(a) approximately 50° C. for approximately 2 minutes
(b) approximately 95° C. for approximately 10 minutes
(c) approximately 95° C. for approximately 15 seconds
(d) approximately 57° C. for approximately 1 minute

The preferred reaction scheme shown to produce specific and sensitive results in the methods of the invention is Stage1: 50° C. for 2 min, Stage2: 95° C. for 10 min, Stage3: 95° C. for 15 sec, 57° C. for 30 sec, 57° C. for 30 sec (=plateau-data collection) for 45 repeats.

It is possible for the methods of the invention to be used in order to detect more than one gene of interest in the same reaction. Through the use of several specific sets of primers, amplification of several nucleic acid targets can be performed in the same reaction mixture. This may be termed “multiplexing”. In a preferred embodiment, one or both primers for each target may be hairpin primers labeled with a fluorescent moiety and a quenching moiety that form a FRET pair. Amplification of several nucleic acid targets requires that a different fluorescent donor and/or acceptor moiety, with a different emission wavelength, be used to label each set of primers. During detection and analysis after an amplification, the reaction mixture is illuminated and read at each of the specific wavelengths characteristic for each of the sets of primers used in the reaction. It can thus be determined which specific target DNAs in the mixture were amplified and labelled. In a specific embodiment, two or more primer pairs for amplification of different respective target sequences are used. Thus the presence and/or amount of a panel of methylated/unmethylated genes of interest can be detected in a single DNA-containing sample

Multiplexing can also be utilised in the context of detecting both the gene of interest and a reference gene in the same reaction. Again, primers labelled with appropriate distinguishable donor and/or acceptor moieties allow the signal generated by amplification of the gene of interest and reference gene respectively to be distinguished.

In one embodiment, a universal quencher is utilised together with suitable fluorophore donors each having a distinguishable emission wavelength maximum. A particularly preferred quencher is DABCYL. Together with DABCYL as quencher, the following fluorophores may each be utilised to allow multiplexing: Coumarin (emission maximum of 475 nm), EDANS (491 nm), fluorescein (515 nm), Lucifer yellow (523 nm), BODIPY (525 nm), Eosine (543 nm), tetramethylrhodamine (575 nm) and texas red (615 nm) (Tyagi et al., Nature Biotechnology, Vol. 16, January 1998; 49-53). Other preferred combinations are discussed herein.

In an alternative embodiment, the DNA-containing sample can be split and the methods of the invention carried out on suitable portions of the sample in order to obtain directly comparable results. Thus, where both the gene of interest and a reference gene are detected, the sample may be split two ways to allow detection of amplification of the gene of interest in real time in one sample portion and detection of amplification of the reference gene in real time in the other sample portion. The sample may be split further to allow suitable control reactions to be carried out, as required. The benefit of this scheme is that a universal FRET pair can be used to label each primer pair and removes the requirement to detect emission at a range of wavelengths. However, this method does rely upon obtaining a suitable sample initially to permit dividing the sample. Whilst any suitable reaction volume may be utilised, in one specific embodiment, the total reaction volume for the amplification step is between approximately 10 and 40 μl, more preferably between approximately 10 and 30 μl and most preferably around 12 μl.

In one aspect, the oligonucleotides, primers or probes, primer pairs, kits or methods of the present invention are used for diagnosing cancer or predisposition of cancer, wherein the presence of unmethylated (or hypomethylated) CTAG1B, CTAG2, PRAME and/or MageA3 in the sample is indicative for cancer or predisposition to cancer. Thus, the present invention provides kits, methods and primers for diagnosing cancer or predisposition to cancer.

“Diagnosis” is defined herein to include screening for a disease or pre-stadia of a disease, identifying a disease or prestadia of a disease, monitoring staging and the state and progression of the disease, checking for recurrence of disease following treatment and monitoring the success of a particular treatment. The tests may also have prognostic value, and this is included within the definition of the term “diagnosis”. The prognostic value of the tests may be used as a marker of potential susceptibility to cancer or as a marker for progression to cancer. Thus patients at risk may be identified before the disease has a chance to manifest itself in terms of symptoms identifiable in the patient. In a preferred embodiment, the cancer is selected from lung cancer, melanoma or breast cancer. In a preferred embodiment, the methods and assays for diagnosis use at least one oligonucleotide comprising, consisting, consisting essentially of, or consisting of the nucleotide sequence of any SEQ ID NO. 1, 2, 3, 4, 5, 6, 7, or 8 for the CTAG1B marker. The methods and assays for diagnosis use at least one oligonucleotide comprising, consisting, consisting essentially of, or consisting of the nucleotide sequence of any SEQ ID NO 9, 10, 11, 12, 13, 14, 15, 16, 17, 18, 19 or for the CTAG2 marker. The methods and assays for diagnosis use at least one oligonucleotide comprising, consisting, consisting essentially of, or consisting of the nucleotide sequence of any SEQ ID NO 20, 21, 22, 23, 24, 25, 26, 27, 28, 29, 30, 31, 32, 33, 34, 35, 36, 37, 38, 39 or 40 for the PRAME marker. In a preferred embodiment, diagnosis of cancer or predisposition to cancer uses oligonucleotides comprising, consisting, consisting essentially of, or consisting of the nucleotide sequence of any SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and 40 and detects the unmethylated form of the gene.

Testing can be performed diagnostically or in conjunction with a therapeutic regimen. RT-PCR assays that establish the predictive value of CTAG1B, CTAG2 and/or PRAME expression in NSCLC, breast cancer or melanoma find their application in the selection of patients suitable for treatment with a CTAG1B, CTAG2, PRAME and/or MageA3 immunotherapeutic. The inventors have shown that an assay designed for the detection of unmethylated CTAG1B, CTAG2, PRAME and/or MageA3 employing oligonucleotides, primers or probes, primer pairs or kits of the invention, can reliably categorize samples as CTAG1B, CTAG2, PRAME and/or MageA3 expressing. The methylation status result obtained with the hypomethylation test is in good concordance with the results obtained with an existing RT-PCR test for CTAG1B, CTAG2 and/or PRAME detection that is used on RNA samples.

Where samples, e.g. fine needle biopsies from non-Small Lung Cancer, are utilised for which qRT-PCR prove difficult, protein expression detected by determining the methylation status of the MageA3 gene provided an valuable alternative. Accordingly, the methylation test has clinical application. Thus, all methods of the invention may be applied to fine needle biopsy samples. In specific embodiments, the methylation status of the MAGE-A3 gene is determined. Such samples as indicated above are unsuitable for carrying out alternative methods of detecting (MAGE-A3) expression. These samples may contain only a few cells and consequentially contain low levels of DNA. For example, such samples may only permit between approximately 70 to 150 μg of input DNA to be employed for each assay. As shown in example 4, the methods of the invention are effective using fine needle biopsy samples.

In a further aspect the invention provides a method of predicting the likelihood of successful treatment of cancer in a subject comprising:

(a) contacting/treating a DNA-containing test sample obtained from a subject with a reagent which selectively modifies unmethylated cytosine residues in the DNA to produce detectable modified residues but which does not modify methylated cytosine residues
(b) amplifying at least a portion of the unmethylated CTAG1B, CTAG2 and/or PRAME gene using at least one primer pair, at least one primer of which is designed to bind only to the sequence of unmethylated DNA respectively following treatment with the reagent, wherein at least one primer in the primer pair comprises, consists essentially of, or consists of the nucleotide sequence of any of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 60 and 61,
(c) determining the methylation status of the CTAG1B, CTAG2, PRAME and/or MageA3 gene;
wherein the presence of unmethylated CTAG1B, CTAG2, PRAME and/or MageA3 in the sample indicates that the likelihood of successful treatment with a CTAG1B, CTAG2, PRAME and/or MageA3 immunotherapeutic is higher than if no or lower levels of unmethylated CTAG1B, CTAG2, PRAME and/or MageA3 gene is detected.

Step (c) involves identifying whether an amplification product has formed. The identification of the amplification product (using any suitable technique as discussed herein) indicates the presence of unmethylated or hypomethylated CTAG1B, CTAG2, PRAME and/or MageA3 in the sample.

Of course, the reverse situation is also applicable and so the methods of the invention may likewise be utilised in order to determine whether there is likely to be resistance to, or unsuccessful treatment using, an CTAG1B, CTAG2, PRAME and/or MageA3 immunotherapeutic agent—the absence of unmethylated CTAG1B, CTAG2, PRAME and/or MageA3 in the sample indicates there is likely to be resistance to treatment and/or that treatment is likely to be unsuccessful. Primers specific for methylated DNA may also be employed in complementary methods, in certain embodiments.

The methods of the invention may also be utilised to select a suitable course of treatment for a patient—the presence of unmethylated CTAG1B, CTAG2, PRAME and/or MageA3 indicates that CTAG1B, CTAG2, PRAME and/or MageA3 immunotherapeutic agents may be beneficially administered, whereas the absence or low level of unmethylated CTAG1B, CTAG2, PRAME and/or MageA3 indicates that immunothereapeutic agents are contra-indicated. The discussion provided in respect of the oligonucleotides, primers or probes, primer pairs, kits or methods of the invention applies to the present aspect mutatis mutandis and all embodiments are therefore envisaged, as appropriate, for this aspect of the invention.

By “likelihood of successful treatment” is meant the probability that treatment of the cancer using any one or more of the listed therapeutic agents, preferably a CTAG1B, CTAG2, PRAME and/or MageA3 immunotherapeutic or a composition comprising CTAG1B, CTAG2, PRAME and/or MageA3, will be successful.

“Resistance” is defined as a reduced probability that treatment of cancer will be successful using any one of the specified immunotherapeutic agents and/or that higher dose will be required to achieve a therapeutic effect.

Hypomethylation of CTAG1B, CTAG2, PRAME and/or MageA3 may be linked to certain cancer types. Accordingly, in a specific embodiment, the invention provides a method of detecting a predisposition to, or the incidence of, breast cancer, lung cancer, including NSCLC or melanoma in a sample comprising detecting the methylation status of the CTAG1B, CTAG2, PRAME and/or MageA3 gene using the oligonucleotides, primers or probes, primer pairs, kits or methods of the invention, wherein detection of unmethylated CTAG1B, CTAG2, PRAME and/or MageA3 in the sample is indicative of a predisposition to, or the incidence of, cancer and in particular melanoma; lung cancer including non-small cell lung carcinoma (NSCLC); or breast cancer. In a further embodiment, the tumour or cancer is selected from prostate cancer, ovarian cancer, breast cancer, bladder cancer; head and neck cancer including oesophagus carcinoma; cervical cancer, colorectal cancer, squamous cell carcinoma; liver cancer; multiple myeloma and colorectal cancer.

In a further aspect, there is provided a method for determining the presence of a CTAG1B, CTAG2, PRAME and/or MageA3 positive tumor comprising detecting the methylation status of the CTAG1B, CTAG2, PRAME and/or MageA3 gene in a sample with use of the oligonucleotides, primers or probes, primer pairs, kits or methods described herein, wherein the presence of unmethylated CTAG1B, CTAG2, PRAME and/or MageA3 is indicative for the presence of a CTAG1B, CTAG2, PRAME and/or MageA3 positive tumor.

Testing can be performed diagnostically or in conjunction with a therapeutic regimen. Testing can also be used to determine what therapeutic or preventive regimen to employ on a patient and be used to monitor efficacy of a therapeutic regimen.

Accordingly, the invention further provides a method for identifying and/or selecting a patient suitable for treatment with a CTAG1B, CTAG2, PRAME and/or MageA3 immunotherapeutic comprising detecting the methylation status of the CTAG1B, CTAG2, PRAME and/or MageA3 gene in a sample of the patient with use of the oligonucleotides, primers or probes, primer pairs, kits or methods described herein, wherein if the CTAG1B, CTAG2, PRAME and/or MageA3 gene is unmethylated the subject is identified and/or selected for treatment with the CTAG1B, CTAG2, PRAME and/or MageA3 immunotherapeutic.

Alternatively, if the gene is not unmethylated the subject is preferably not selected for treatment with a CTAG1B, CTAG2, PRAME and/or MageA3 immunotherapeutic.

In a related aspect, the invention provides a method for predicting the likelihood of successful treatment of cancer comprising detecting the methylation status of the CTAG1B, CTAG2, PRAME and/or MageA3 gene in a sample of the patient with use of the oligonucleotides, primers or probes, primer pairs, kits or methods described herein, wherein if the gene is unmethylated the likelihood of successful treatment with a CTAG1B, CTAG2, PRAME and/or MageA3 immunotherapeutic is higher than if the gene is methylated.

Alternatively, the absence of unmethylated CTAG1B, CTAG2, PRAME and/or MageA3 in the sample indicates that the likelihood of resistance to treatment with a CTAG1B, CTAG2, PRAME and/or MageA3 immunotherapeutic is higher than if the gene is unmethylated. Thus, the detection of a methylated CTAG1B, CTAG2, PRAME and/or MageA3 gene (or lack of detection of the hypomethylated gene) indicates that the probability of successful treatment with an immunotherapeutic is low.

Thus, the patient population may be selected for treatment on the basis of their methylation status with respect to the CTAG1B, CTAG2, PRAME and/or MageA3 gene. This leads to a much more focussed and personalised form of medicine and thus leads to improved success rates since patients will be treated with drugs which are most likely to be effective.

In a further related aspect, the invention provides a method of selecting a suitable treatment regimen for cancer comprising detecting the methylation status of the CTAG1B, CTAG2, PRAME and/or MageA3 gene in a sample of the patient with use of the oligonucleotides, primers or probes, primer pairs, kits or methods described herein, wherein if the gene is unmethylated, an immunotherapeutic (in particular a CTAG1B, CTAG2, PRAME and/or MageA3 immunotherapeutic) is selected for treatment.

Alternatively, if the gene is not unmethylated, treatment with an immunotherapeutic is contra-indicated.

Also provided is a method of treating cancer in a subject comprising administration of an immunotherapeutic, wherein the subject has been selected for treatment on the basis of measuring the methylation status of a CTAG1B, CTAG2, PRAME and/or MageA3 gene, according to any of the methods of the invention or by using an oligonucleotide, primer or probe, primer pair, kit or a method as described herein. Preferably, for all of the different aspects described herein, the detection of unmethylated CTAG1B, CTAG2, PRAME and/or MageA3 gene corresponds to an increased level of CTAG1B, CTAG2, PRAME and/or MageA3 protein.

CTAG1B, CTAG2, MAGE-A3 and/or PRAME immunotherapeutics, useful in the present invention, include CTAG1B, CTAG2, MAGE-A3 and/or PRAME based compositions. Examples of compositions comprising CTAG1B, CTAG2, MAGE-A3 and/or PRAME include compositions comprising full length CTAG1B, CTAG2, MAGE-A3 or PRAME, substantially full-length CTAG1B, CTAG2, MAGE-A3 and/or PRAME and fragments of CTAG1B, CTAG2, MAGE-A3 or PRAME, for example peptides of CTAG1B, CTAG2, MAGE-A3 or PRAME.

Examples of peptides that may be used in the present invention include the following MAGE-A3 peptides:

| SEQ ID NO | Peptide sequence |
|---|---|
| SEQ ID NO: 64 | FLWGPRALV |
| SEQ ID NO: 65 | EVDPIGHLY |
| SEQ ID NO: 66 | MEVDPIGHLY |
| SEQ ID NO: 67 | VHFLLLKYRA |
| SEQ ID NO: 68 | LVHFLLLKYR |
| SEQ ID NO: 69 | LKYRAREPVT |
| SEQ ID NO: 70 | ACYEFLWGPRALVETS |
| SEQ ID NO: 71 | TQHFVQENYLEY |

The MAGE protein may be full length MAGE-A3 or may comprise a substantially full-length fragment of MAGE3, for example amino acids 3-314 of MAGE3 (312 amino acids in total), or other MAGE-A3 fragments in which between 1 and 10 amino acids are deleted from the N-terminus and/or C-terminus of the MAGE-A3 protein.

In one embodiment, the CTAG1B, CTAG2, MAGE-A3 and/or PRAME protein, fragment or peptide may be linked to a fusion partner protein.

Examples of antigens that may be used in the present invention include the following CTAG1B, CTAG2 and/or PRAME antigens:

By way of example, suitable peptides of CTAG1B include peptides with binding motifs to HLA-A2, such as peptides 157-167, 157-165 and 155-163, as disclosed in Jager, et al., Proc. Natl. Acad. Sci. USA, 97(22):12198-12203 (2000). Peptides of CTAG1B may include one or more MHC Class 1 or Class 2 epitopes e.g. those known as A31, DR1, DR2, DR4, DR7, DP4, B35, B51, Cw3, Cw6 and A2 (see WO2008/089074).

By way of example, peptides of PRAME may include peptides with binding motifs to HLA-A2, such as peptides LYVDSLFFL, ALYVDSLFFL, VLDGLDVLL, SLYSFPEA and SLLQHILGL (see Quintarelli et al, Blood, 1 Sep. 2008, Vol. 112, No. 5, pp. 1876-1885). Peptides of PRAME may include one or more MHC Class 1 or Class 2 epitopes.

By way of example, peptides of CTAG2 may include ELVRRILSR, MLMAQEALAFL, SLLMWITQC, LAAQERRVPR, as disclosed in Sun et al Cancer Immunology, Immunotherapy Volume 55, Number 6/June, 2006.

The CTAG1B, CTAG2, MAGE-A3 and/or PRAME protein, fragment or peptide and fusion partner protein may be chemically conjugated, or may be expressed as a recombinant fusion protein. In an embodiment in which the antigen and partner are expressed as a recombinant fusion protein, this may allow increased levels to be produced in an expression system compared to non-fused protein. Thus the fusion partner protein may assist in providing T helper epitopes (immunological fusion partner protein), preferably T helper epitopes recognised by humans, and/or assist in expressing the protein (expression enhancer protein) at higher yields than the native recombinant protein. In one embodiment, the fusion partner protein may be both an immunological fusion partner protein and expression enhancing partner protein.

In one embodiment of the invention, the immunological fusion partner protein that may be used is derived from protein D, a surface protein of the gram-negative bacterium, Haemophilus influenza B (WO 91/18926) or a derivative thereof. The protein D derivative may comprise the first ⅓ of the protein, or approximately the first ⅓ of the protein. In one embodiment, the first N-terminal 109 residues of protein D may be used as a fusion partner to provide a CTAG1B, CTAG2, MAGE-A3 and/or PRAME antigen with additional exogenous T-cell epitopes and increase expression level in E. coli (thus acting also as an expression enhancer). In an alternative embodiment, the protein D derivative may comprise the first N-terminal 100-110 amino acids or approximately the first N-terminal 100-110 amino acids. In one embodiment, the protein D or derivative thereof may be lipidated and lipoprotein D may be used: the lipid tail may ensure optimal presentation of the antigen to antigen presenting cells. In an alternative embodiment, the protein D or derivative thereof is not lipidated. The “secretion sequence” or “signal sequence” of protein D, refers to approximately amino acids 1 to 16, 17, 18 or 19 of the naturally occurring protein. In one embodiment, the secretion or signal sequence of protein D refers to the N-terminal 19 amino acids of protein D. In one embodiment, the secretion or signal sequence is included at the N-terminus of the protein D fusion partner. As used herein, the “first third (⅓)”, “first 109 amino acids” and “first N-terminal 100-110 amino acids” refer to the amino acids of the protein D sequence immediately following the secretion or signal sequence. Amino acids 2-K and 3-L of the signal sequence may optionally be substituted with the amino acids 2-M and 3-D.

In one embodiment, the antigen for immunotherapy may be in a fusion with protein D in the form of a construct such as: Protein D-CTAG1B-His, or Protein D-CTAG2-His, or Protein D-PRAME-His, or Protein D-MAGE-A3-His. This fusion protein may comprise the signal sequence of protein D, amino acids 1 to 109 of Protein D, the antigen for immunotherapy (which may be a full length or partial protein sequence), a spacer and a polyhistidine tail (His) that may facilitate the purification of the fusion protein during the production process, for example:

i) An 18-residue signal sequence and the first N-terminal 109 residues of protein D;
ii) Two unrelated residues (methionine and aspartic acid);
iii) the antigen for immunotherapy;
iv) Two glycine residues functioning as a hinge region; and
v) seven Histidine residues.

The portion of protein D that may be employed preferably does not include the secretion sequence or signal sequence. The fusion partner protein comprises amino acids Met-Asp-Pro at or within the N-terminus of the fusion protein sequence and does not include the secretion sequence or the signal sequence of protein D in certain embodiments. For example the fusion partner protein may comprise or consist of approximately or exactly amino acids 17 to 127, 18 to 127, 19 to 127 or 20 to 127 of protein D.

By way of example, suitable PRAME antigens based on fusions proteins with protein D are described in WO2008/087102 which document is incorporated herein by reference in its entirety.

In another embodiment the immunological fusion partner protein may be the protein known as LytA or a protein derived therefrom. LytA is derived from Streptococcus pneumoniae which synthesise an N-acetyl-L-alanine amidase, amidase LytA, (coded by the LytA gene (Gene, 43 (1986) page 265-272)) an autolysin that specifically degrades certain bonds in the peptidoglycan backbone. The C-terminal domain of the LytA protein is responsible for the affinity to choline or to some choline analogues such as DEAE. This property has been exploited for the development of E. coli C-LytA expressing plasmids useful for expression of fusion proteins. Purification of hybrid proteins containing the C-LytA fragment at its amino terminus has been described (Biotechnology: 10, (1992) page 795-798). In one embodiment, the C terminal portion of the molecule may be used. The repeat portion of the LytA molecule found in the C terminal end starting at residue 178 may be utilised. In one embodiment, the LytA portion may incorporate residues 188-305.

Other fusion partners include the non-structural protein from influenzae virus, NS1 (hemagglutinin). In one embodiment, the N terminal 81 amino acids of NS1 are utilised, although different fragments may be used provided they include T-helper epitopes.

In one embodiment of the present invention, the antigen for use in immunotherapy may comprise a derivatised free thiol. In one embodiment of the present invention, the MAGE-A3 protein may comprise a derivatised free thiol. Such antigens have been described in WO99/40188. In particular carboxyamidated or carboxymethylated derivatives may be used.

Antigens for immunotherapy may be used in combination with other immunotherapeutic antigens, in the form of fusions or in admixture For example, CTAG1B may be employed as a fusion with CTAG2, or a fragment thereof, see WO2008/089074 which document is incorporated herein by reference in its entirety.

Combinations of immunotherapeutic agents, either as fusion proteins or admixtures, include any combination of 2 or more of CTAG1B, CTAG2, PRAME or MAGE3, for example, although the invention is not restricted to these specific antigens, and any immunotherapeutic antigen can be used. By way of example, MAGE-3 antigens have, for example, been described as suitable to be formulated in combination with NY-ESO-1; see WO2005/105139, which document is incorporated herein by reference in its entirety.

In a further embodiment immunotherapeutics for use in the present invention may comprises a nucleic acid molecule encoding an immunotherapeutic protein, fragment or peptide or fusion protein as described herein. In one embodiment of the present invention, the sequences may be inserted into a suitable expression vector and used for DNA/RNA vaccination. Microbial vectors expressing the nucleic acid may also be used as vector-delivered immunotherapeutics.

Examples of suitable viral vectors include retroviral, lentiviral, adenoviral, adeno-associated viral, herpes viral including herpes simplex viral, alpha-viral, pox viral such as Canarypox and vaccinia-viral based systems. Gene transfer techniques using these viruses are known to those skilled in the art. Retrovirus vectors for example may be used to stably integrate the polynucleotide of the invention into the host genome, although such recombination is not preferred. Replication-defective adenovirus vectors by contrast remain episomal and therefore allow transient expression. Vectors capable of driving expression in insect cells (for example baculovirus vectors), in human cells, yeast or in bacteria may be employed in order to produce quantities of the protein encoded by the polynucleotides of the present invention, for example for use as subunit vaccines or in immunoassays.

In a preferred embodiment the adenovirus used as a live vector is a replication defective simian adenovirus. Typically these viruses contain an E1 deletion and can be grown on cell lines that are transformed with an E1 gene. Preferred Simian adenoviruses are viruses isolated from Chimpanzee. In particular C68 (also known as Pan 9) (See U.S. Pat. No. 6,083,716) and Pan 5, 6 and Pan 7 (W0 03/046124) are preferred for use in the present invention. These vectors can be manipulated to insert a heterologous gene of the invention such that the gene product may be expressed. The use, formulation and manufacture of such recombinant adenoviral vectors is set forth in detail in WO 03/046142.

Conventional recombinant techniques for obtaining nucleic acid sequences, and production of expression vectors are described in Maniatis et al., Molecular Cloning—A Laboratory Manual; Cold Spring Harbor, 1982-1989.

For protein based compositions, the proteins of the present invention may be provided either soluble in a liquid form or in a lyophilised form.

Each human dose may comprise 1 to 1000 μg of protein. In one embodiment, the dose may comprise 30-300 μg of protein.

The composition for immunotherapy as described herein may further comprise a vaccine adjuvant, and/or an immunostimulatory cytokine or chemokine. Thus, the term “immunotherapeutic” applied herein may incorporate all compositions for immunotherapy as described herein, including suitable adjuvants.

Suitable vaccine adjuvants for use in the present invention are commercially available such as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminium salts such as aluminium hydroxide gel (alum) or aluminium phosphate; salts of calcium, iron or zinc; an insoluble suspension of acylated tyrosine; acylated sugars; cationically or anionically derivatised polysaccharides; polyphosphazenes; biodegradable microspheres; monophosphoryl lipid A and quil A. Cytokines, such as GM-CSF or interleukin-2, -7, or -12, and chemokines may also be used as adjuvants.

In one embodiment, the adjuvant may comprise a combination of monophosphoryl lipid A, such as 3-de-O-acylated monophosphoryl lipid A (3D-MPL) together with an aluminium salt. Alternatively, the adjuvant may comprise 3D-MPL or other toll like receptor 4 (TLR4) ligands such as aminoalkyl glucosaminide phosphates as disclosed in WO 98/50399, WO 01/34617 and WO 03/065806.

Another adjuvant that may be used is a saponin, for example QS21 (Aquila Biopharmaceuticals Inc., Framingham, Mass.), that may be used alone or in combination with other adjuvants. For example, in one embodiment, there is provided a combination of a monophosphoryl lipid A and saponin derivative, such as the combination of QS21 and 3D-MPL as described in WO 94/00153, or a composition in which the QS21 is quenched with cholesterol, as described in WO 96/33739. Other suitable formulations comprise an oil-in-water emulsion and tocopherol. In one embodiment, the adjuvant comprises QS21, 3D-MPL and tocopherol in an oil-in-water emulsion, as described in WO 95/17210.

Other adjuvants for use in the present invention may comprise TLR9 antagonists such as unmethylated CpG containing oligonucleotides, in which the CpG dinucleotide is unmethylated. Such oligonucleotides are well known and are described in, for example WO 96/02555.

Suitable oligonucleotides for use in the present invention (in this context) may include:

| | | |
|---|---|---|
| SEQ 56 | TCC ATG ACG TTC CTG ACG TT | CpG 1826 |
| SEQ 57 | TCT CCC AGC GTG CGC CAT | CpG 1758 |
| SEQ 58 | ACC GAT GAC GTC GCC GGT GAC | |
| | GGC ACC ACG | |
| SEQ 59 | TCG TCG TTT TGT CGT TTT GTC | CpG 2006, |
| | GTT | CpG 7909 |
| SEQ 60 | TCC ATG ACG TTC CTG ATG CT | CpG 1668 |

CpG-containing oligonucleotides may also be used alone or in combination with other adjuvants. For example, in one embodiment, the adjuvant comprises a combination of a CpG-containing oligonucleotide and a saponin derivative particularly the combination of CpG and QS21 as disclosed in WO 00/09159 and WO 00/62800.

Accordingly there is provided a composition comprising an immunotherapeutic as described herein, wherein the adjuvant comprises one or more of 3D-MPL, QS21, a CpG oligonucleotide, a polyethylene ether or ester or a combination of two or more of these adjuvants. The immunotherapeutic component within the composition may be presented in an oil in water or a water in oil emulsion vehicle or in a liposomal formulation, in certain embodiments.

In one embodiment, the adjuvant may comprise one or more of 3D-MPL, QS21 and an immunostimulatory CpG oligonucleotide. In an embodiment all three adjuvant components are present. The components may be either presented in a liposomal formulation or an oil in water emulsion, such as described in WO 95/17210.

In another embodiment 3D MPL- and Qs21 are presented in an oil in water emulsion, and in the absence of a CpG oligonucleotide.

The amount of 3D-MPL used is generally small, but depending on the formulation may be in the region of 1-1000 μg per dose, preferably 1-500 μg per dose, and more preferably between 1 to 100 μg per dose.

The amount of CpG or immunostimulatory oligonucleotides in the adjuvants of the present invention is generally small, but depending on the formulation may be in the region of 1-1000 μg per dose, preferably 1-500 μg per dose, and more preferably between 1 to 100 μg per dose.

The amount of saponin for use in the adjuvants of the present invention may be in the region of 1-1000 μg per dose, preferably 1-500 μg per dose, more preferably 1-250 μg per dose, and most preferably between 1 to 100 μg per dose.

The adjuvant formulations as described herein may additionally comprise an oil in water emulsion and/or tocopherol or may be formulated in a liposomal composition.

Other suitable adjuvants include Montanide ISA 720 (Seppic, France), SAF (Chiron, Calif., United States), ISCOMS (CSL), MF-59 (Chiron), Ribi Detox, RC-529 (GSK, Hamilton, Mont.) and other aminoalkyl glucosaminide 4-phosphates (AGPs).

Generally, each human dose may comprise 0.1-1000 μg of antigen, for example 0.1-500 μg, 0.1-100 μg, or 0.1 to 50 μg. An optimal amount for a particular immunotherapeutic can be ascertained by standard studies involving observation of appropriate immune responses in vaccinated subjects. Following an initial vaccination, subjects may receive one or several booster immunisation adequately spaced.

Alternatively, a composition for use in the method of the present invention may comprise a pharmaceutical composition comprising an immunotherapeutic as described herein in combination with a pharmaceutically acceptable excipient.

The invention will now be described with respect to the following non-limiting examples:

### DESCRIPTION OF THE FIGURES

FIG. 1: Localization of CTAG1B assays on the chromosome. One potential transcription start site (TSS) has been described. Beginning of the gene structure is represented as follows: open boxes represent a 300 bp promoter region followed by a vertical line indicating the potential TSS and then the solid boxes represent the first exons. Assaylocations are indicated with open boxes followed by their corresponding names, “(U)” indicating an Unmethylation specific assay (i.e. an assay specific for hypomethylation). The number of CpG count is spotted over a region of 20 kb.

FIG. 2: Localization of CTAG2 assays on the chromosome. One potential transcription start site (TSS) has been described. Beginning of the gene structure is represented as follows: open boxes represent a 300 bp promoter region followed by a vertical line indicating the potential TSS and then the solid boxes represent the first exons. Assay locations are indicated with open boxes followed by their corresponding names, “(U)” indicating an Unmethylation specific assay (i.e. an assay specific for hypomethylation). The number of CpG count is spotted over a region of 20 kb. CTAG2 was also named CTAG2_{—}1.

FIG. 3: Localization of PRAME assays on the chromosome. One potential transcription start site (TSS) has been described. Beginning of the gene structure is represented as follows: the open box represents a 300 bp promoter region followed by a vertical line indicating the potential TSS and then the solid boxes represent the first exons. Assay locations are indicated with open boxes followed by their corresponding names, “(U)” indicating an Unmethylation specific assay (i.e. an assay specific for hypomethylation). The number of CpG count is spotted over a region of 20 kb.

FIG. 4: Localization of one MAGE-A3 assay (MAGE-A3 GO_{—}2 U assay) on the chromosome. One potential transcription start site (TSS) has been described. Beginning of the gene structure is represented as follows: the open box represents a 300 bp promoter region followed by a vertical line indicating the potential TSS and then the solid boxes represent the first exons. Assay location is indicated with an open box associated to its corresponding names, “(U)” indicating an Unmethylation specific assay (i.e. an assay specific for hypomethylation). The number of CpG count is spotted over a region of 20 kb.

FIG. 5: Schematic overview of the Amplifluor® technique.

At least one primer (forward primer) in the primer pair contains a “hairpin” structure carrying a donor (FAM) and an acceptor moiety (DABCYL) of a molecular energy transfer pair. This figure describes the case of a sense primer also called forward primer (for primer) which is linked to the hairpin labelled structure. In the absence of amplification, fluorescence emitted by the donor moiety is effectively accepted by the acceptor moiety leading to quenching of fluorescence. During amplification, the primer is incorporated into an amplification product. During the second round of amplification the stem loop or hairpin structure is disrupted. The acceptor moiety is no longer capable of effectively quenching the fluorescence emitted by the donor moiety. Thus, the donor moiety produces a detectable fluorescence signal.

FIG. 6: Concordance between real time MS-PCR results obtained with CTAG1B_{—}1S demethylation assay and CTAG1B (gene alias NY-ESO-1) real time RT-PCR expression data for 48 Non-small Cell Lung Cancer (NSCLC) samples. The vertical solid thick line indicates the cut-off for the demethylation assay and the horizontal thick line the expression cut-off. The linear regression is represented by a black line; R=0.35

FIG. 7: Concordance between real time MS-PCR results obtained with CTAG2_{—}1_AS demethylation assay and CTAG2 (gene alias LAGE-1) real time RT-PCR expression data for 48 Non-small Cell Lung Cancer (NSCLC) samples. The vertical solid thick line indicates the cut-off for the demethylation assay and the horizontal thick line the expression cut-off. The linear regression is represented by a black line; R=0.86; Concordance between the two techniques is 87.5%.

FIG. 8: Concordance between real time MS-PCR results obtained with CTAG2_{—}1_S demethylation assay and CTAG2 (gene alias LAGE-1) real time RT-PCR expression data for 48 Non-small Cell Lung Cancer (NSCLC) samples. The vertical solid thick line indicates the cut-off for the demethylation assay and the horizontal thick line the expression cut-off. The linear regression is represented by a black line; R=0.88; Concordance between the two techniques is 87.5%.

FIG. 9: Concordance between real time MS-PCR results obtained with CTAG2_{—}2_S demethylation assay and CTAG2 (gene alias LAGE-1) real time RT-PCR expression data for 48 Non-small Cell Lung Cancer (NSCLC) samples. The vertical solid thick line indicates the cut-off for the demethylation assay and the horizontal thick line the expression cut-off. The linear regression is represented by a black line; R=0.47; Concordance between the two techniques is 68.8%.

FIG. 10: Concordance between real time MS-PCR results obtained with CTAG2_{—}3_S demethylation assay and CTAG2 (gene alias LAGE-1) real time RT-PCR expression data for 48 Non-small Cell Lung Cancer (NSCLC) samples. The vertical solid thick line indicates the cut-off for the demethylation assay and the horizontal thick line the expression cut-off. The linear regression is represented by a black line; R=0.55; Concordance between the two techniques is 66.7%.

FIG. 11: Concordance between real time MS-PCR results obtained with PRAME_{—}7_S demethylation assay and PRAME real time RT-PCR expression data for 48 Non-small Cell Lung Cancer (NSCLC) samples. The vertical solid thick line indicates the cut-off for the demethylation assay and the horizontal thick line the expression cut-off. The linear regression is represented by a black line; R=0.62; Concordance between the two techniques is 83.3%.

FIG. 12: Concordance between real time MS-PCR results obtained with PRAME_{—}1_AS demethylation assay and PRAME real time RT-PCR expression data for 48 Non-small Cell Lung Cancer (NSCLC) samples. The vertical solid thick line indicates the cut-off for the demethylation assay and the horizontal thick line the expression cut-off. The linear regression is represented by a black line; R=0.39; Concordance between the two techniques is 66.7%.

FIG. 13: Concordance between real time MS-PCR results obtained with PRAME_{—}2_S demethylation assay and PRAME real time RT-PCR expression data for 48 Non-small Cell Lung Cancer (NSCLC) samples. The vertical solid thick line indicates the cut-off for the demethylation assay and the horizontal thick line the expression cut-off. The linear regression is represented by a black line; R=0.64; Concordance between the two techniques is 79.2%.

FIG. 14: Concordance between real time MS-PCR results obtained with PRAME_{—}3_AS demethylation assay and PRAME real time RT-PCR expression data for 48 Non-small Cell Lung Cancer (NSCLC) samples. The vertical solid thick line indicates the cut-off for the demethylation assay and the horizontal thick line the expression cut-off. The linear regression is represented by a black line; R=0.73; Concordance between the two techniques is 85.4%.

FIG. 15: Concordance between real time MS-PCR results obtained with PRAME_{—}6_AS demethylation assay and PRAME real time RT-PCR expression data for 48 Non-small Cell Lung Cancer (NSCLC) samples. The vertical solid thick line indicates the cut-off for the demethylation assay and the horizontal thick line the expression cut-off. The linear regression is represented by a black line; R=0.24; Concordance between the two techniques is 66.7%.

FIG. 16: Concordance between real time MS-PCR results obtained with CTAG1B_{—}1_S demethylation assay and CTAG1B (gene alias NY-ESO-1) real time RT-PCR expression data for 31 Melanoma samples; The vertical solid thick line indicates the cut-off for the demethylation assay and the horizontal thick line the expression cut-off. The linear regression is represented by a black line.

FIG. 17: Concordance between real time MS-PCR results obtained with CTAG2_{—}1_AS demethylation assay and CTAG2 (gene alias LAGE-1) real time RT-PCR expression data for 31 Melanoma samples. The vertical solid thick line indicates the cut-off for the demethylation assay and the horizontal thick line the expression cut-off. The linear regression is represented by a black line; R=0.63; Concordance between the two techniques is 74.2%.

FIG. 18: Concordance between real time MS-PCR results obtained with CTAG2_{—}1_S demethylation assay and CTAG2 (gene alias LAGE-1) real time RT-PCR expression data for 31 Melanoma samples. The vertical solid thick line indicates the cut-off for the demethylation assay and the horizontal thick line the expression cut-off. The linear regression is represented by a black line; R=0.62; Concordance between the two techniques is 74.2%.

FIG. 19: Concordance between real time MS-PCR results obtained with CTAG2_{—}2_S demethylation assay and CTAG2 (gene alias LAGE-1) real time RT-PCR expression data for 31 Melanoma samples. The vertical solid thick line indicates the cut-off for the demethylation assay and the horizontal thick line the expression cut-off. The linear regression is represented by a black line; R=0.34; Concordance between the two techniques is 51.6%.

FIG. 20: Concordance between real time MS-PCR results obtained with CTAG2_{—}3_S demethylation assay and CTAG2 (gene alias LAGE-1) real time RT-PCR expression data for 31 Melanoma samples. The vertical solid thick line indicates the cut-off for the demethylation assay and the horizontal thick line the expression cut-off. The linear regression is represented by a black line; R=0.58; Concordance between the two techniques is 74.2%.

FIG. 21: Concordance between real time MS-PCR results obtained with PRAME_{—}7_S demethylation assay and PRAME real time RT-PCR expression data for 31 Melanoma samples. The vertical solid thick line indicates the cut-off for the demethylation assay and the horizontal thick line the expression cut-off. The linear regression is represented by a black line; R=0.70; Concordance between the two techniques is 90.3%.

FIG. 22: Concordance between real time MS-PCR results obtained with PRAME_{—}1_AS demethylation assay and PRAME real time RT-PCR expression data for 31 Melanoma samples. The vertical solid thick line indicates the cut-off for the demethylation assay and the horizontal thick line the expression cut-off. The linear regression is represented by a black line; R=0.26; Concordance between the two techniques is 93.5%.

FIG. 23: Concordance between real time MS-PCR results obtained with PRAME_{—}2_S demethylation assay and PRAME real time RT-PCR expression data for 31 Melanoma samples. The vertical solid thick line indicates the cut-off for the demethylation assay and the horizontal thick line the expression cut-off. The linear regression is represented by a black line; R=0.29; Concordance between the two techniques is 90.3%.

FIG. 24: Concordance between real time MS-PCR results obtained with PRAME_{—}3_AS demethylation assay and PRAME real time RT-PCR expression data for 31 Melanoma samples. The vertical solid thick line indicates the cut-off for the demethylation assay and the horizontal thick line the expression cut-off. The linear regression is represented by a black line; R=0.48; Concordance between the two techniques is 90.3%.

FIG. 25: Concordance between real time MS-PCR results obtained with PRAME_{—}6_AS demethylation assay and PRAME real time RT-PCR expression data for 31 Melanoma samples. The vertical solid thick line indicates the cut-off for the demethylation assay and the horizontal thick line the expression cut-off. The linear regression is represented by a black line; R=0.51; Concordance between the two techniques is 90.3%.

FIG. 26: Concordance between real time MS-PCR results obtained with CTAG2_{—}1_AS demethylation assay and CTAG2 real time RT-PCR expression data for 28 breast cancer samples The vertical solid thick line indicates the cut-off for the demethylation assay and the horizontal thick line the expression cut-off. The linear regression is represented by a black line; R=0.56; Concordance between the two techniques is 71.4%.

FIG. 27: Concordance between real time MS-PCR results obtained with CTAG2_{—}3_S demethylation assay and CTAG2 real time RT-PCR expression data for 28 breast cancer samples. The vertical solid thick line indicates the cut-off for the demethylation assay and the horizontal thick line the expression cut-off. The linear regression is represented by a black line; R=0.34; Concordance between the two techniques is 78.6%.

### EXPERIMENTAL SECTION

We investigated the methylation status of different CT genes in non small cell lung cancer (NSCLC), melanoma and breast cancer with unmethylated assays for different purposes such as screening, detection, staging, prognosis, prediction and monitoring of disease, prediction and monitoring of treatments (including vaccine options).

### Example 1

### CT-Antigen Assays: In-Silico Primer Design

Several hypomethylation assays, designed to target the unmethylated version of the gene sequence were developed for the CT-antigens CTAG1B (=NY-ESO-1), CTAG2 (=LAGE-1) and PRAME.

Primer3 software adapted to MSP requirements (http://fokker.wi.mit.edu/primer3/input.htm) was used for the in silico design of forward (F) and reverse (R) primers for detecting the unmethylated form of CTAG1B, CTAG2 and PRAME. Conditions were as follows: amplicon size: 50-120; primer size: 19-27; melting temp: 55-65; max 3′ self complementarity=0; Window of 4000 bp around TSS (number to return=2000).

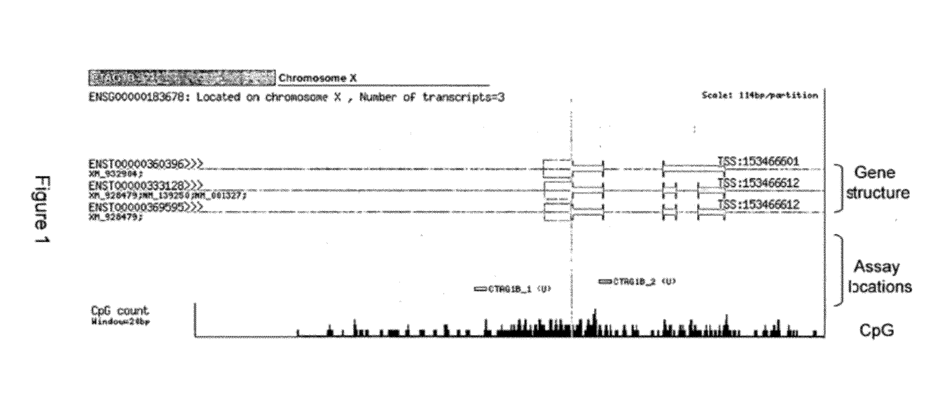

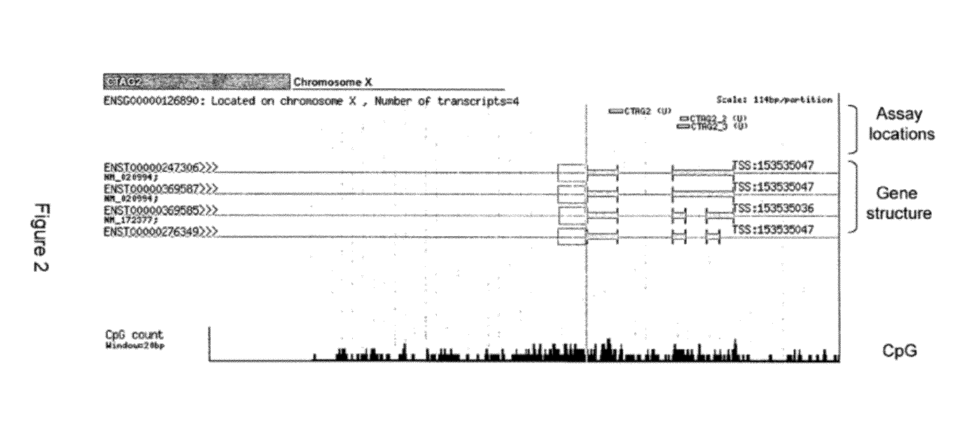

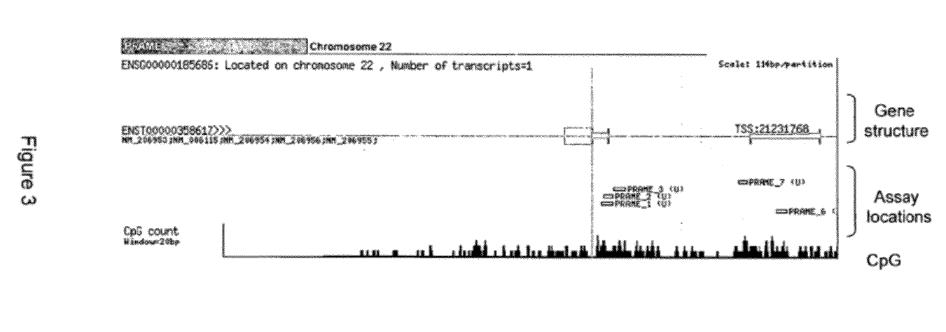

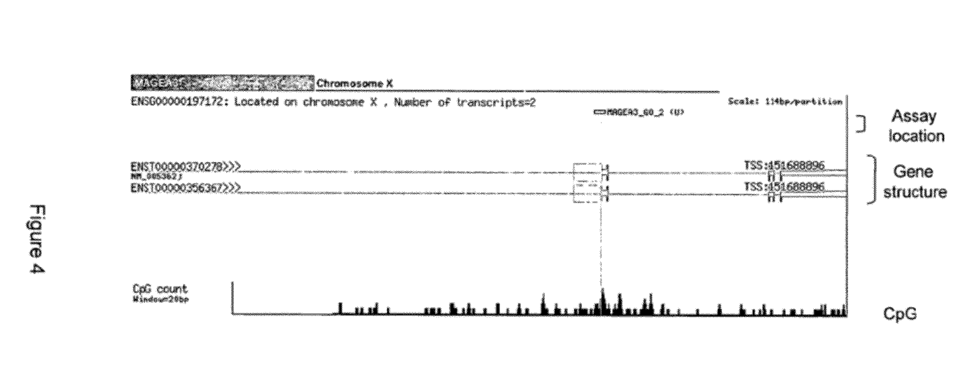

Location of the CTAG1B, CTAG2 and PRAME U-primers relative to the Transcription Start Site (TSS) is shown in FIG. 1, FIG. 2 and FIG. 3 respectively. The region of investigated included the promoter area and extended to the first exons.

Either the forward or reverse primer was synthesised to incorporate a suitable stem loop or hairpin structure carrying a donor and acceptor moiety at the 5′ end carrying the sequence: 5′AGCGATGCGTTCGAGCATCGCU 3′ (SEQ ID NO 43.)

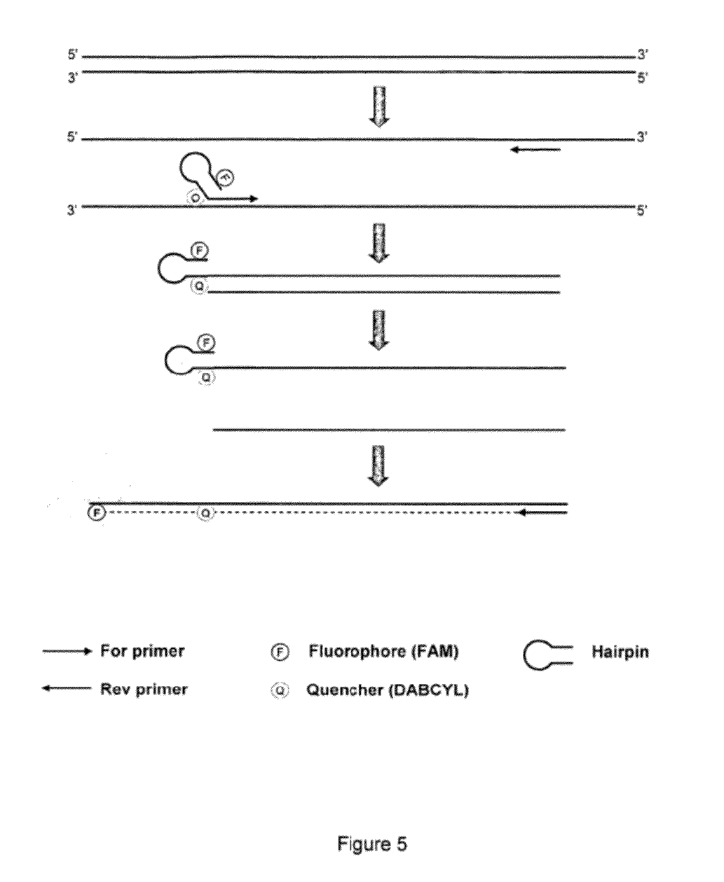

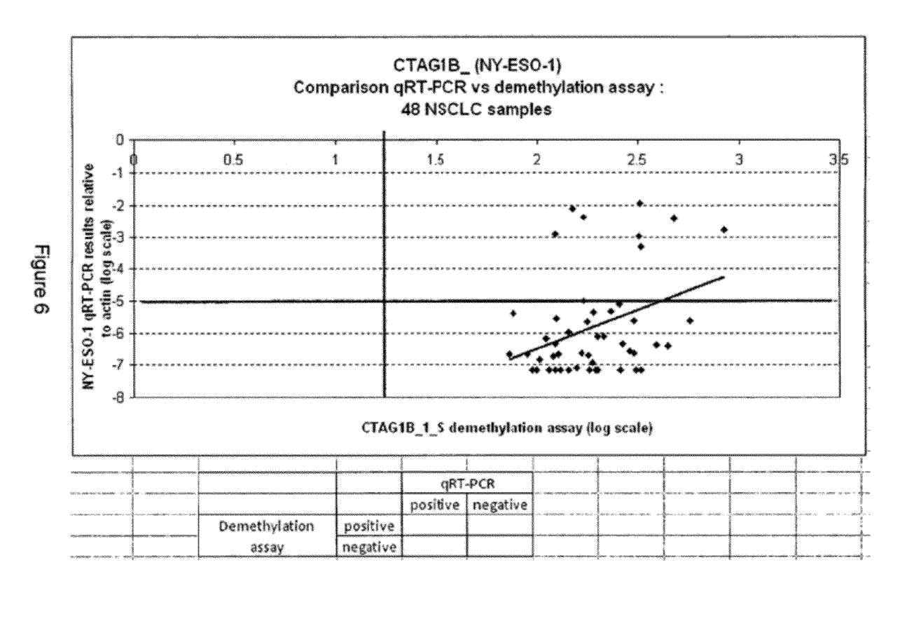

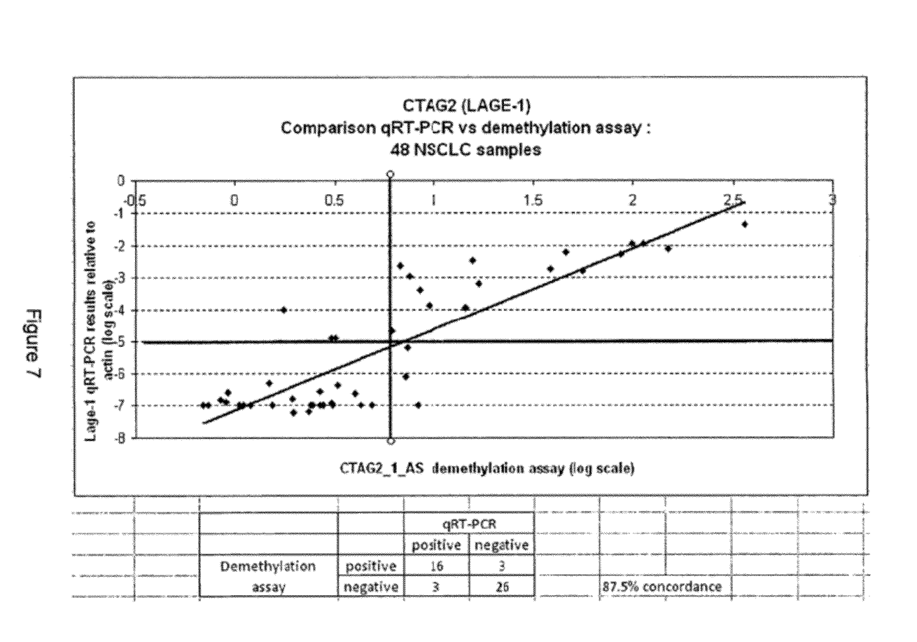

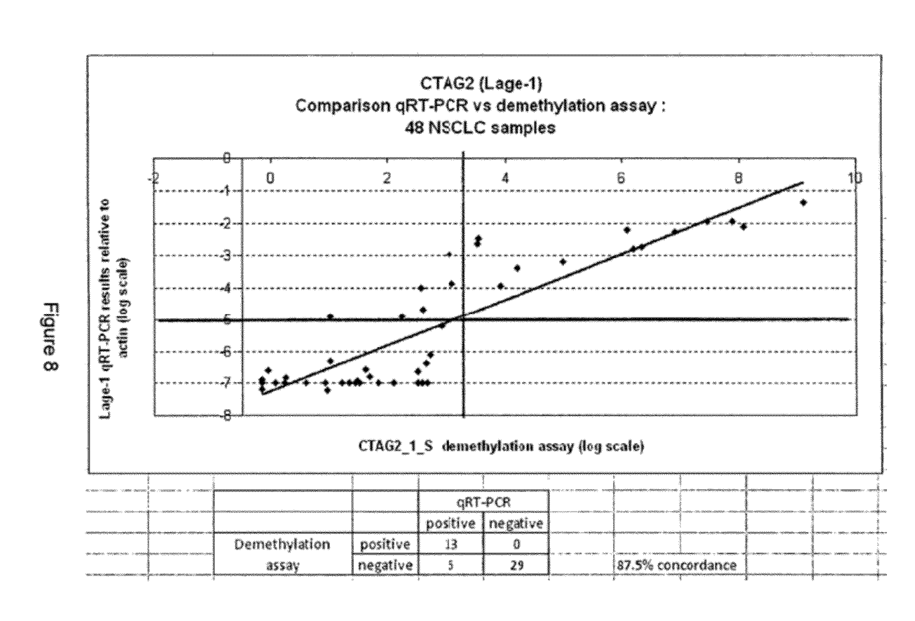

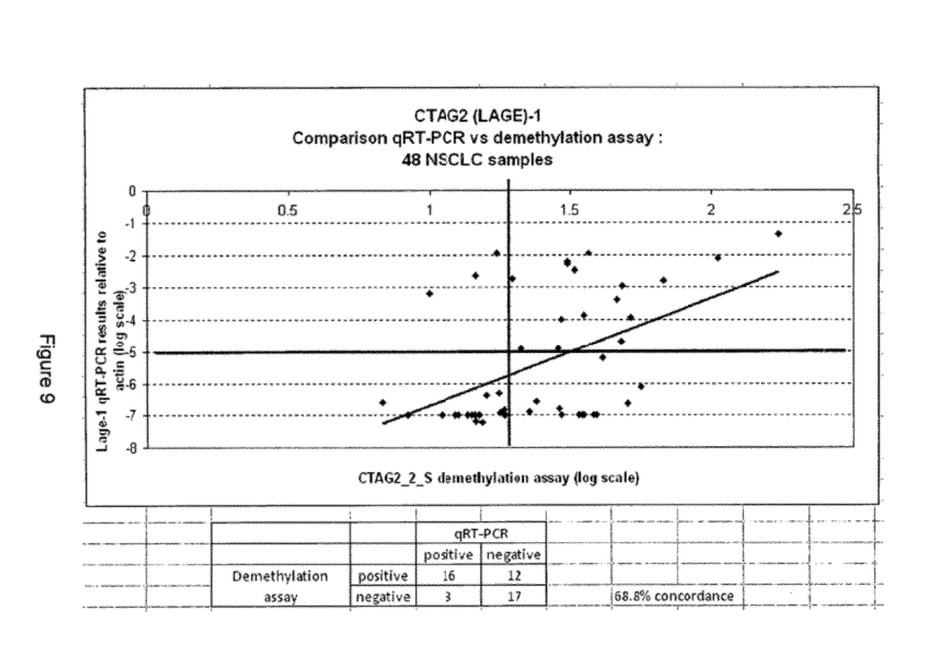

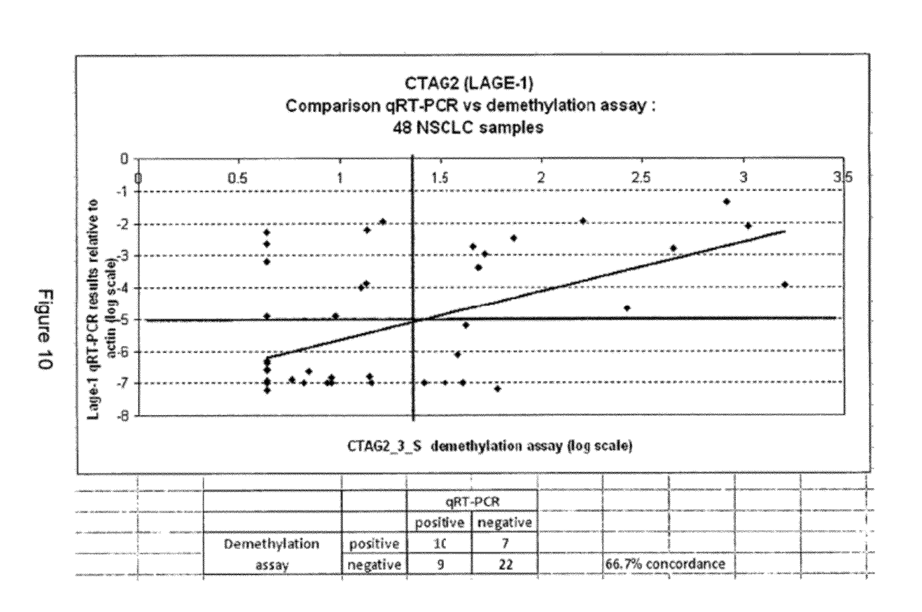

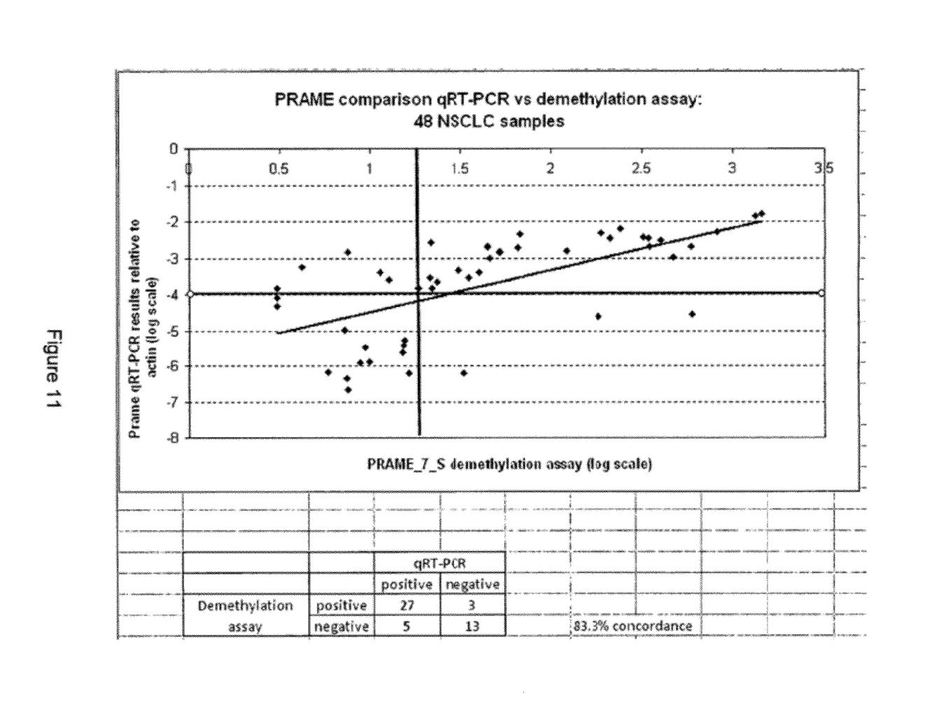

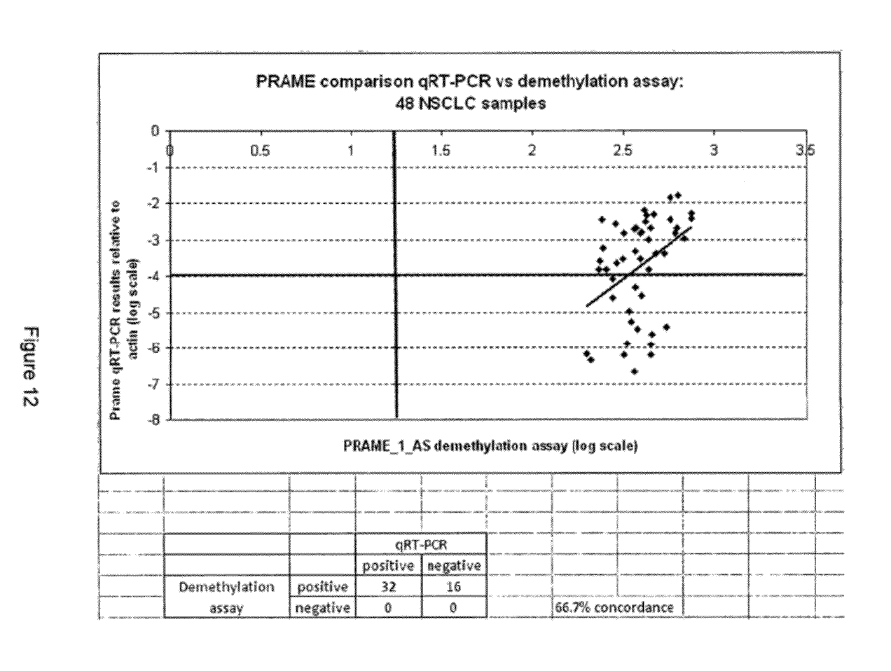

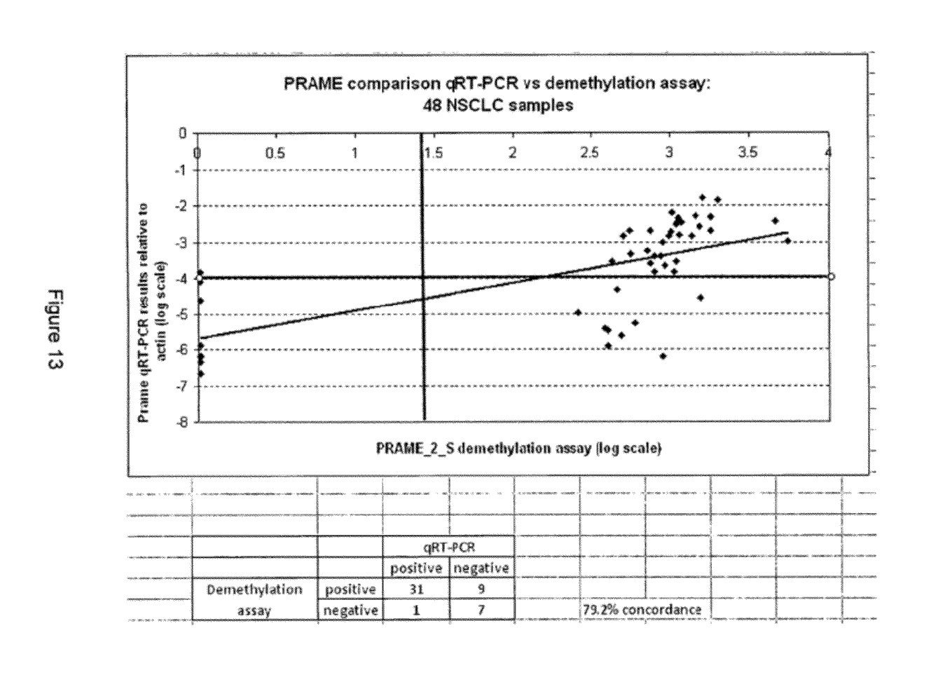

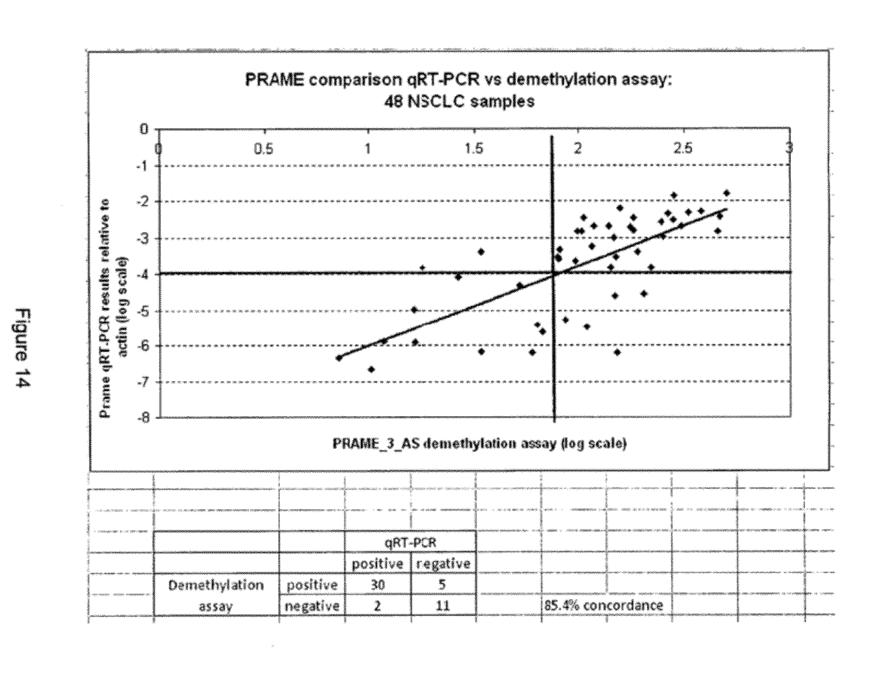

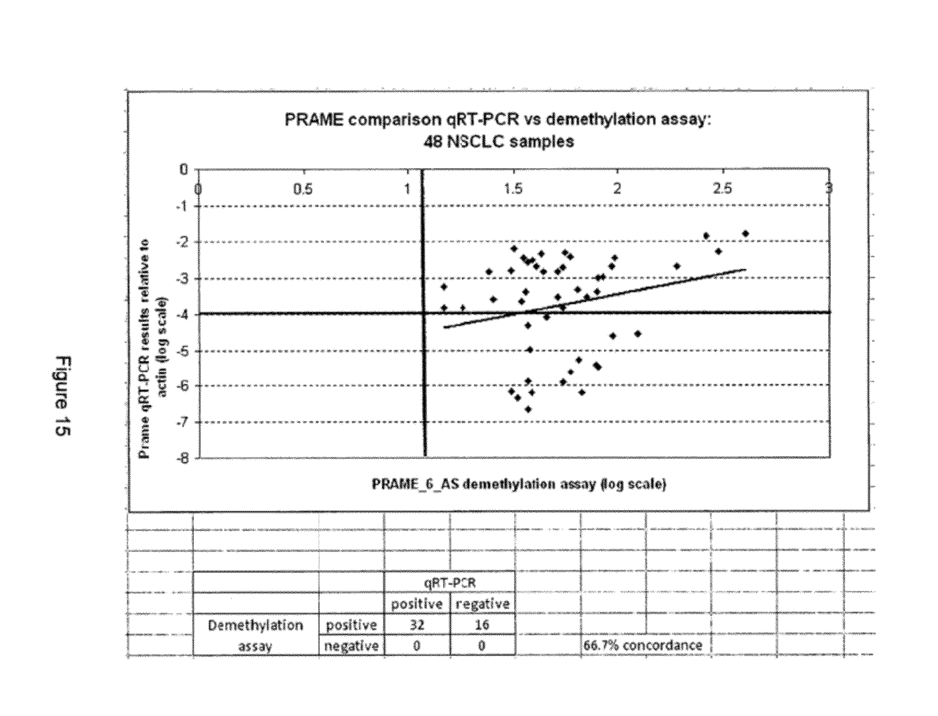

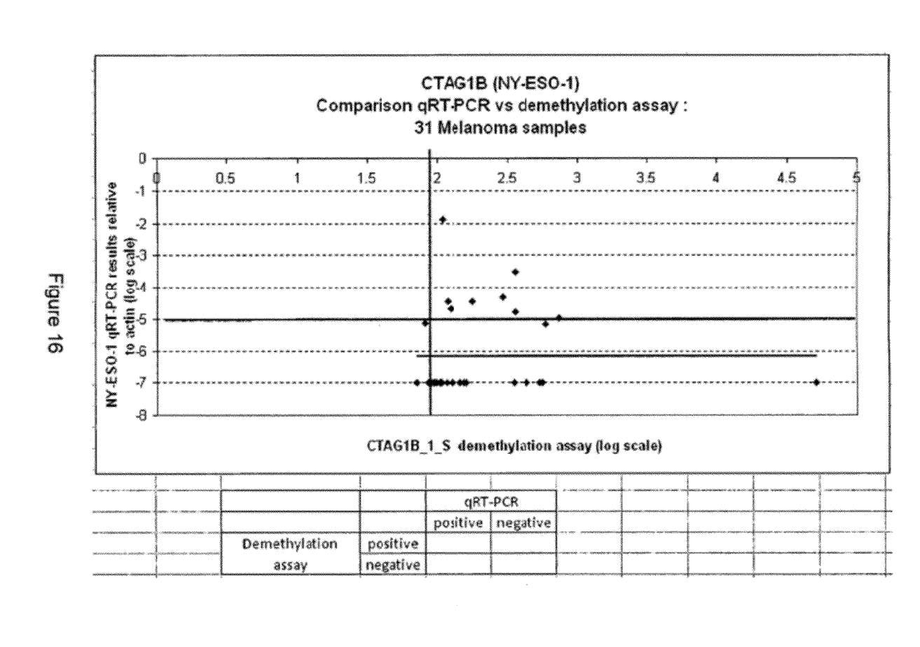

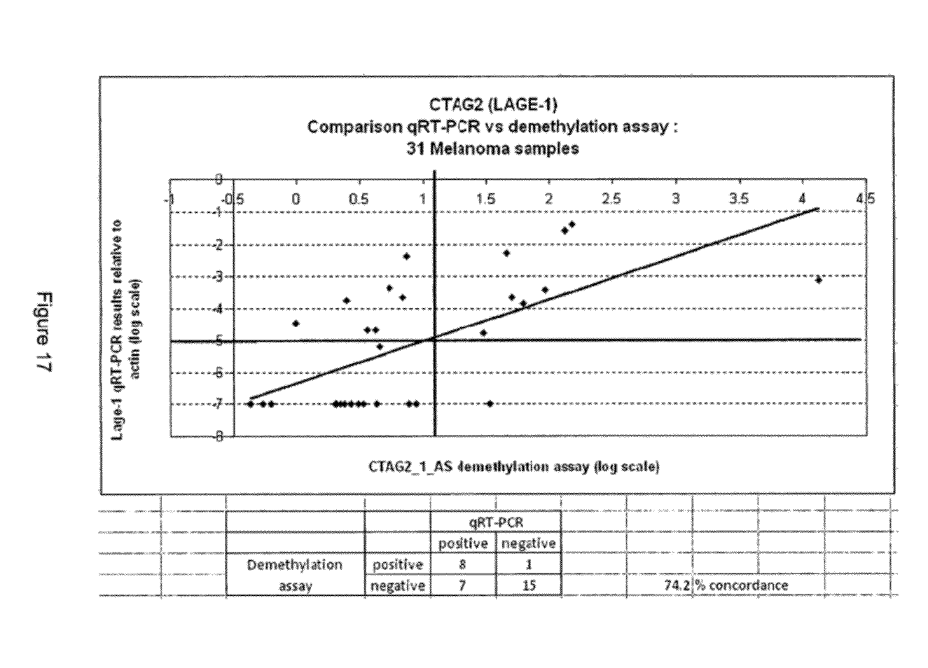

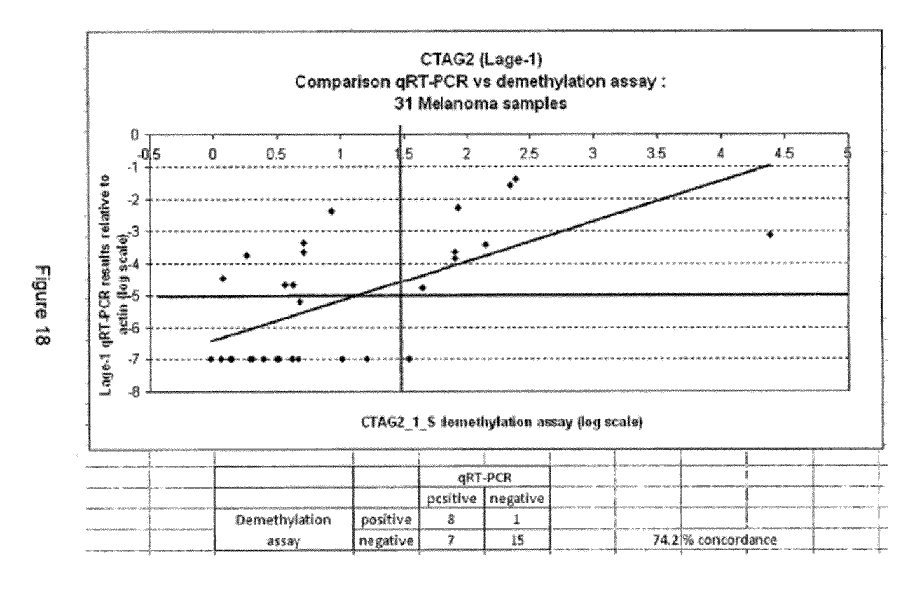

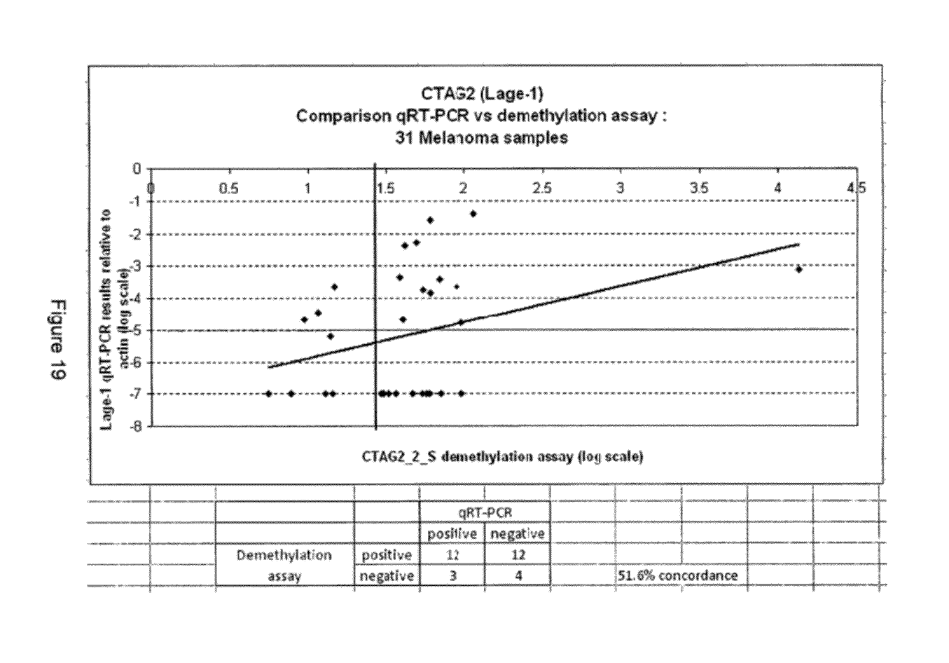

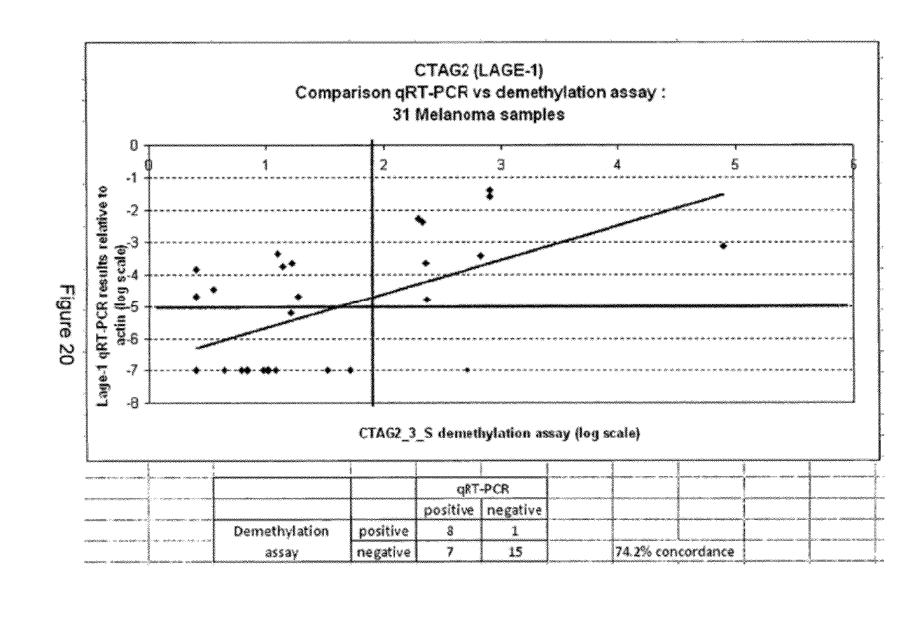

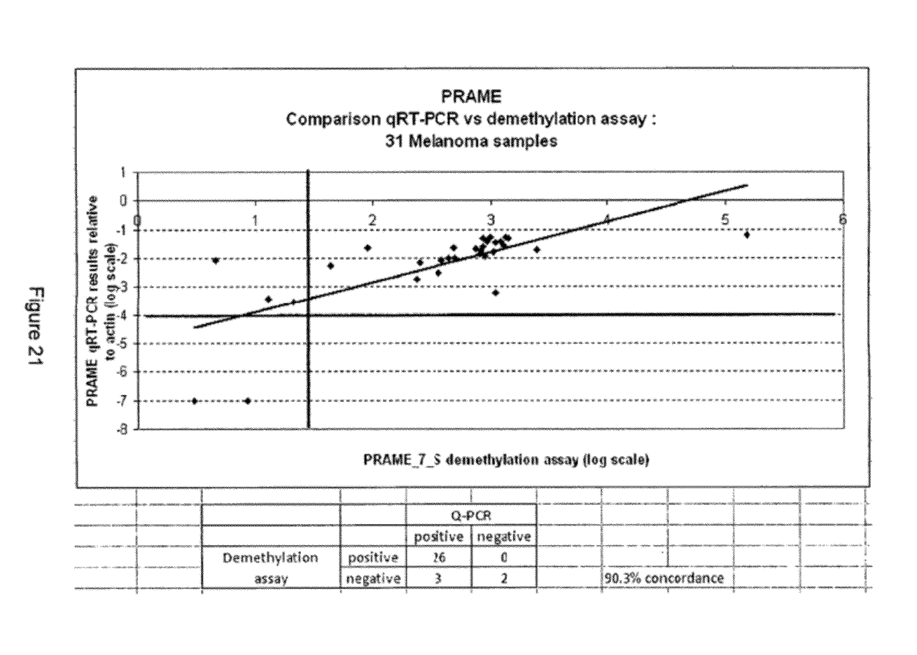

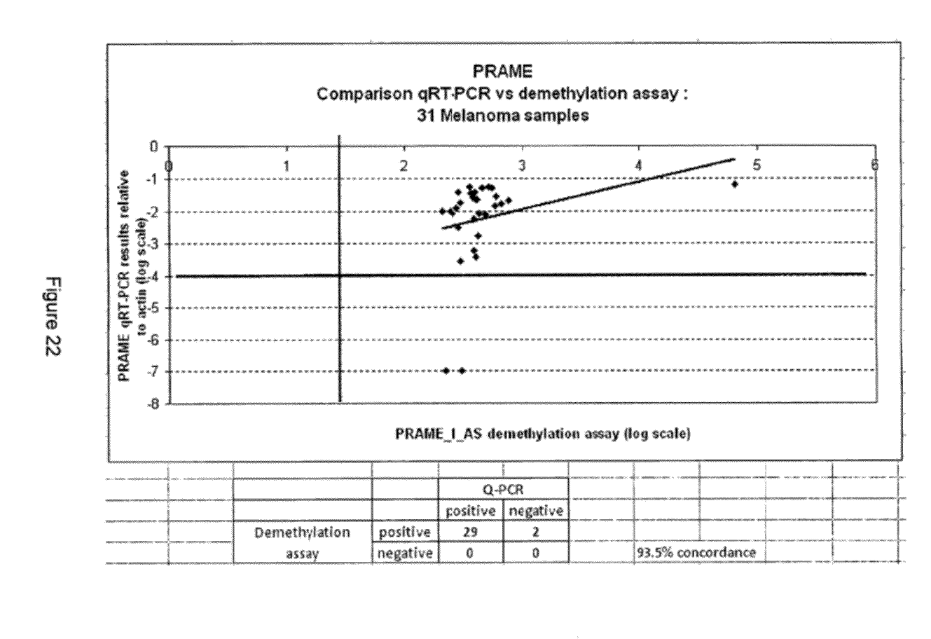

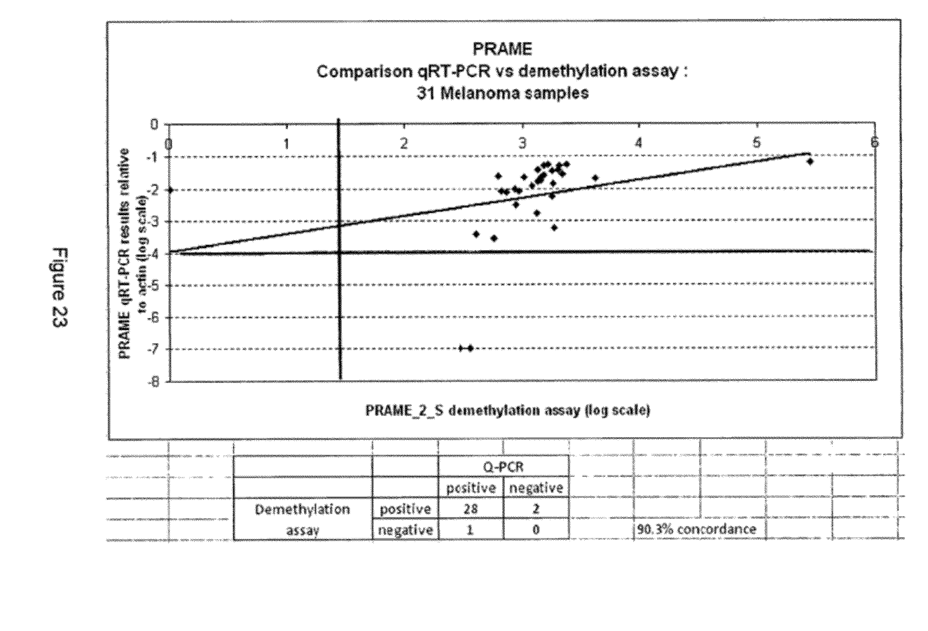

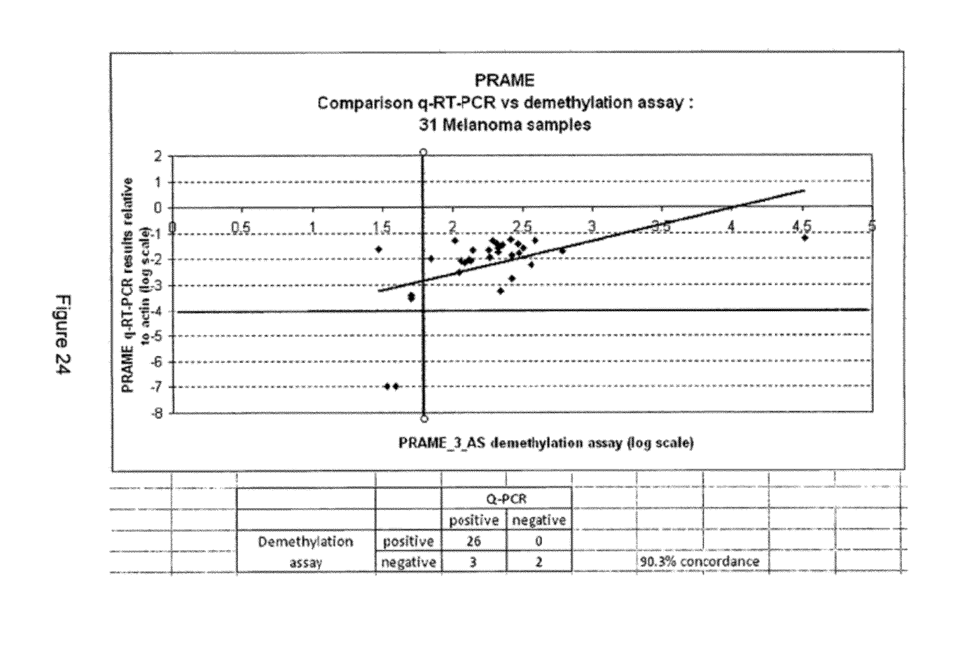

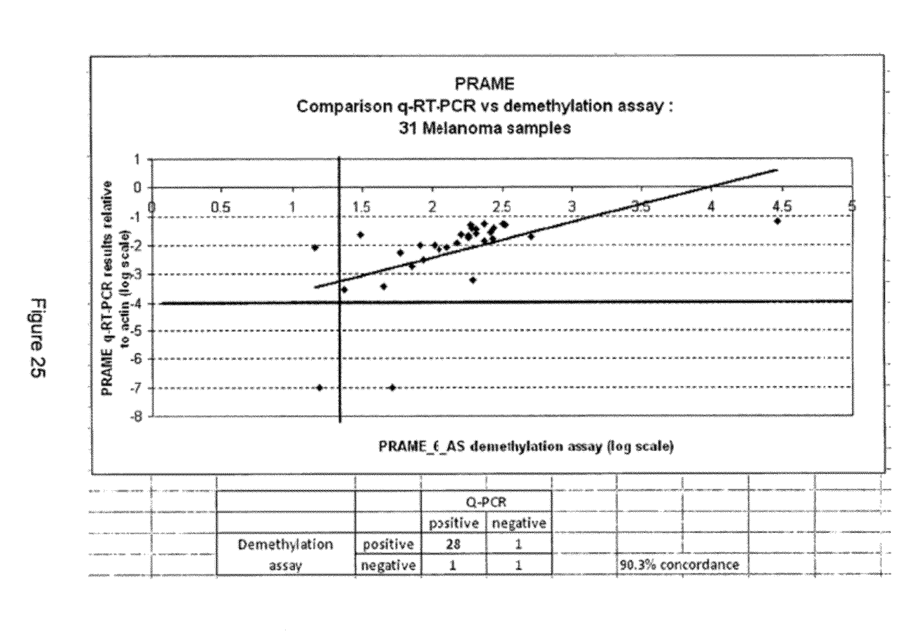

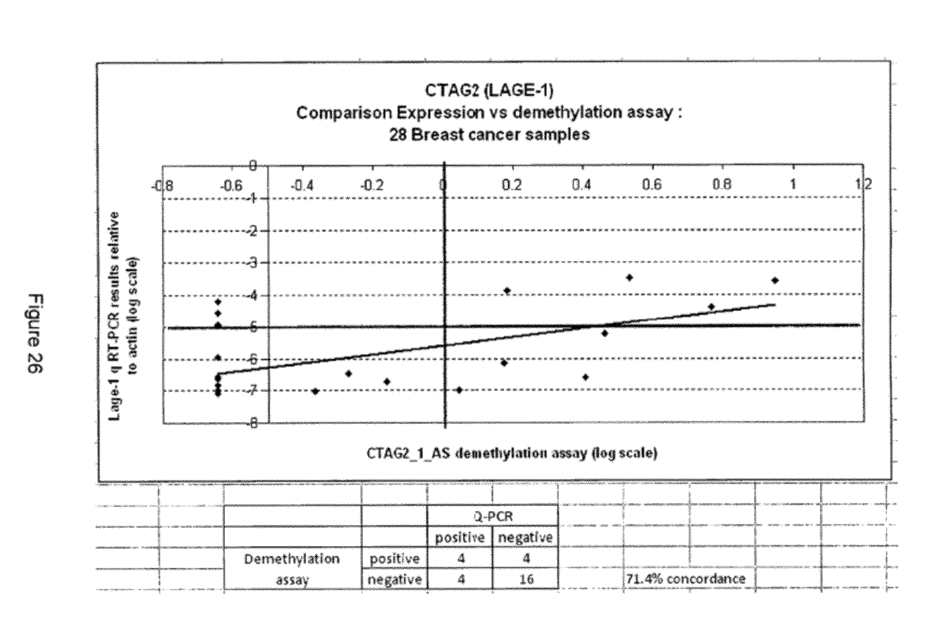

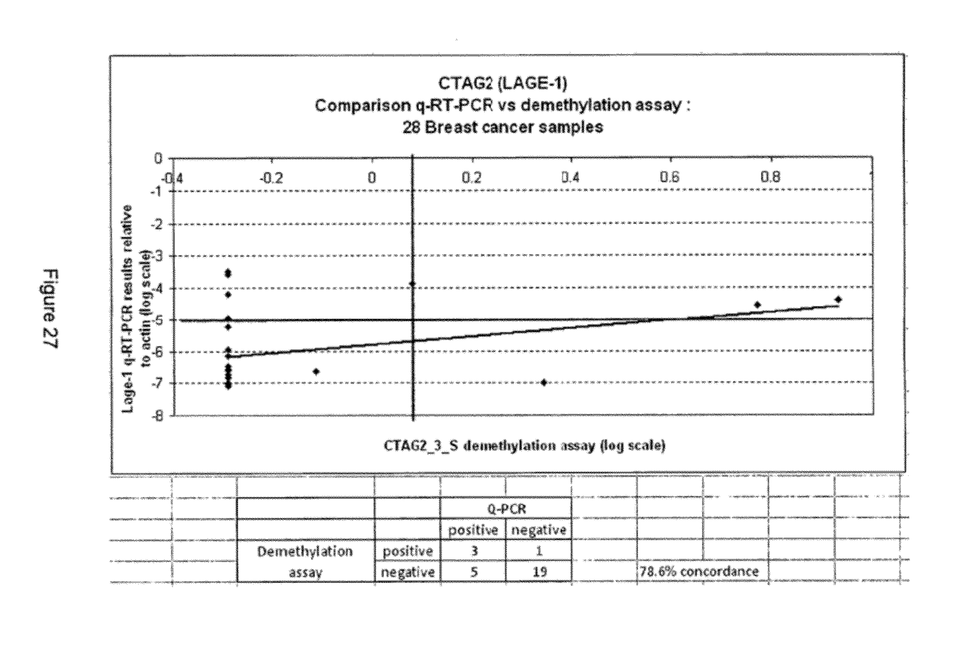

The different primer combinations as tested are set out in Table 1 below. Technology principle of PCR using amplifluor primers is described in FIG. 5.

TABLE 1
Primer and amplifluor detector sequences CTAG1B, CTAG2,
PRAME, MageA3 and ACTB. Amplifluor moiety is underlined.

| | | | 5′ to 3′ Sequences |
|---|---|---|---|
| Assay name | | Type of | Detector Modifications: |
| Amplicon length | Primer names | primers | 5′ FAM and internal dUdabcyl |
| CTAG1B_1_S_A | CTAG1B_1_S_A | Amplifluor | AGCGATGCGTTCGAGCATCGCUGGAAGG |
| MP | MP | sense | TGGGGGAGAGTG |
| 151 bp | | primer | (SEQ ID NO. 1) |
| | CTAG1B_1_AS | antisense | AAAACAACACAACCCCAAAAA |
| | | primer | (SEQ ID NO. 2) |
| CTAG1B_1_AS_ | CTAG1B_1_AS_ | Amplifluor | AGCGATGCGTTCGAGCATCGCUAAAACA |
| AMP | AMP | anti sense | ACACAACCCCAAAAA |
| 151 bp | | primer | (SEQ ID NO. 3) |
| | CTAG1B_1_S | Sense | GGAAGGTGGGGGAGAGTG |
| | | primer | (SEQ ID NO. 4) |
| CTAG1B_2_S_A | CTAG1B_2_S_A | Amplifluor | AGCGATGCGTTCGAGCATCGCUGGGTTG |
| MP | MP | sense | GAGAGTTGTTTGTTTG |
| 152 bp | | primer | (SEQ ID NO. 5) |
| | CTAG1B_2_AS | antisense | CACATCTCCCCCACCTCCT |
| | | primer | (SEQ ID NO. 6) |
| CTAG1B_2_AS_ | CTAG1B_2_AS_ | Amplifluor | AGCGATGCGTTCGAGCATCGCUCACATC |
| AMP | AMP | anti sense | TCCCCCACCTCCT |
| 152 bp | | primer | (SEQ ID NO. 7) |
| | CTAG1B_2_S | Sense | GGGTTGGAGAGTTGTTTGTTTG |
| | | primer | (SEQ ID NO. 8) |
| CTAG2_1_S_AM | CTAG2_1_S_AM | Amplifluor | AGCGATGCGTTCGAGCATCGCUTGGTGG |
| P | P | sense | TGTTGTTTTTGTGT |
| 172 bp | | primer | (SEQ ID NO. 9) |
| | CTAG2_1_AS | antisense | CTTAACCCTATTATCTCCATCTC |
| | | primer | (SEQ ID NO. 10) |
| CTAG2_1_AS_A | CTAG2_1_AS_A | Amplifluor | AGCGATGCGTTCGAGCATCGCUCTTAAC |
| MP | MP | anti sense | CCTATTATCTCCATCTC |
| 172 bp | | primer | (SEQ ID NO. 11) |
| | CTAG2_1_S | Sense | TGGTGGTGTTGTTTTTGTGT |
| | | primer | (SEQ ID NO. 12) |
| CTAG2_2_S_AM | CTAG2_2_S_AM | Amplifluor | AGCGATGCGTTCGAGCATCGCUGGTGGT |
| P | P | sense | TTTGAAGGATTTTATTG |
| 102 bp | | primer | (SEQ ID NO. 13) |
| | CTAG2_2_AS | antisense | ACCCAACACCTTCCCTATCCT |
| | | primer | (SEQ ID NO. 14) |
| CTAG2_2_AS_A | CTAG2_2_AS_A | Amplifluor | AGCGATGCGTTCGAGCATCGCUACCCAA |
| MP | MP | anti sense | CACCTTCCCTATCCT |
| 102 bp | | primer | (SEQ ID NO. 15) |
| | CTAG2_2_S | Sense | GGTGGTTTTGAAGGATTTTATTG |
| | | primer | (SEQ ID NO. 16) |
| CTAG2_3_S_AM | CTAG2_3_S_AM | Amplifluor | AGCGATGCGTTCGAGCATCGCUTTTTGTT |
| P | P | sense | TTGGGATGTTGTATTTT |
| 147 bp | | primer | (SEQ ID NO. 17) |
| | CTAG2_3_AS | antisense | CCTCATCCACCCAACACCTT |
| | | primer | (SEQ ID NO. 18) |
| CTAG2_3_AS_A | CTAG2_3_AS_A | Amplifluor | AGCGATGCGTTCGAGCATCGCUCCTCAT |
| MP | MP | anti sense | CCACCCAACACCTT |
| 147 bp | | primer | (SEQ ID NO. 19) |
| | CTAG2_3_S | Sense | TTTTGTTTTGGGATGTTGTATTTT |
| | | primer | (SEQ ID NO. 20) |
| PRAME_1_S_AM | PRAME_1_S_AM | Amplifluor | AGCGATGCGTTCGAGCATCGCUTGGGTT |
| P | P | sense | TGTAGTGTTTTAGTATTGTTT |
| 143 bp | | primer | (SEQ ID NO. 21) |
| | PRAME_1_AS | antisense | TCCACCCTACTTTCCCTACATTC |
| | | primer | (SEQ ID NO. 22) |
| PRAME_1_AS_A | PRAME_1_AS_A | Amplifluor | AGCGATGCGTTCGAGCATCGCUTCCACC |
| MP | MP | anti sense | CTACTTTCCCTACATTC |
| 143 bp | | primer | (SEQ ID NO. 23) |
| | PRAME_1_S | Sense | TGGGTTTGTAGTGTTTTAGTATTGTTT |
| | | primer | (SEQ ID NO. 24) |
| PRAME_2_S_AM | PRAME_2_S_AM | Amplifluor | AGCGATGCGTTCGAGCATCGCUTTGTTTT |
| P | P | sense | GGGATATTTTATTTGTTTT |
| 128 bp | | primer | (SEQ ID NO. 25) |
| | PRAME_2_AS | antisense | AAAAACTCCACCCTACTTTCC |
| | | primer | (SEQ ID NO. 26) |
| PRAME_2_AS_A | PRAME_2_AS_A | Amplifluor | AGCGATGCGTTCGAGCATCGCUAAAAAC |
| MP | MP | anti sense | TCCACCCTACTTTCC |
| 128 bp | | primer | (SEQ ID NO. 27) |
| | PRAME_2_S | Sense | TTGTTTTGGGATATTTTATTTGTTTT |
| | | primer | (SEQ ID NO. 28) |
| PRAME_3_S_AM | PRAME_3_S_AM | Amplifluor | AGCGATGCGTTCGAGCATCGCUGAGGG |
| P | P | sense | GAGGGGTGTGAATGTG |
| 141 bp | | primer | (SEQ ID NO. 29) |
| | PRAME_3_AS | antisense | CATTCCTCCCTACTCCCAAAAA |
| | | primer | (SEQ ID NO. 30) |
| PRAME_3_AS_A | PRAME_3_AS_A | Amplifluor | AGCGATGCGTTCGAGCATCGCUCATTCC |
| MP | MP | anti sense | TCCCTACTCCCAAAAA |
| 141 bp | | primer | (SEQ ID NO. 31) |
| | PRAME_3_S | Sense | GAGGGGAGGGGTGTGAATGTG |
| | | primer | (SEQ ID NO. 32) |
| PRAME_6_S_AM | PRAME_6_S_AM | Amplifluor | AGCGATGCGTTCGAGCATCGCUTGGTGG |
| P | P | sense | ATGTTTTGGGATTT |
| 129 bp | | primer | (SEQ ID NO. 33) |
| | PRAME_6_AS | antisense | CAACATTTCTACCTCTACTCCCACCTT |
| | | primer | (SEQ ID NO. 34) |
| PRAME_6_AS_A | PRAME_6_AS_A | Amplifluor | AGCGATGCGTTCGAGCATCGCUCAACAT |
| MP | MP | anti sense | TTCTACCTCTACTCCCACCTT |
| 129 bp | | primer | (SEQ ID NO. 35) |
| | PRAME_6_S | Sense | TGGTGGATGTTTTGGGATTT |
| | | primer | (SEQ ID NO. 36) |
| PRAME_7_S_AM | PRAME_7_S_AM | Amplifluor | AGCGATGCGTTCGAGCATCGCUGTTTTG |
| P | P | sense | GAAGGATTGAGAAATGG |
| 120 bp | | primer | (SEQ ID NO. 37) |
| | PRAME_7_AS | antisense | CACCCTAACCACTACATAAAACAAA |
| | | primer | (SEQ ID NO. 38) |
| PRAME_7_AS_A | PRAME_7_AS_A | Amplifluor | AGCGATGCGTTCGAGCATCGCUCACCCT |
| MP | MP | anti sense | AACCACTACATAAAACAAA |
| 120 bp | | primer | (SEQ ID NO. 39) |
| | PRAME_7_S | Sense | GTTTTGGAAGGATTGAGAAATGG |
| | | primer | (SEQ ID NO. 40) |
| ACTB_S_AMP | ACTB_S_AMP | Amplifluor | AGCGATGCGTTCGAGCATCGCUTAGGGA |
| 125 bp | | sense | GTATATAGGTTGGGGAAGTT |
| | | primer | (SEQ ID NO. 41) |
| | ACTB_AS | antisense | AACACACAATAACAAACACAAATTCAC |
| | | primer | (SEQ ID NO. 42) |
| MAGE-A3 GO_2 | MAGEA3_GO_2_ | Amplifluor | AGCGATGCGTTCGAGCATCGCUTGGAAT |
| U | U_F_AMP | sense | TTAGGGTAGTATTGT |
| 140 bp | | primer | (SEQ ID NO. 61) |
| | MAGEA3_GO_2_ | antisense | CCCTCCACCAACATCAAA |
| | U_AS | primer | (SEQ ID NO. 62) |

### Example 2

### Temperature Gradient & Assay Selection

### Material and Methods

Temperature gradient for standard material preparation: PCR were performed using a MJ Research PTC-200 thermocycler. The cycling conditions were as followed: stage 1, 5 min at 95° C.; stage 2 for 35 repeats 30 s at 95° C., 30 s at annealing temperature, 30 s at 72° C.; stage 3, 30 min at 72° C., hold at 4° C. The annealing temperature was set across the heating bloc of the instrument as a gradient from 57° C. to 62° C.

Cloning: PCR products were ligated into TOPO® TA vectors (TOPO® cloning kit, Invitrogen®) and One Shot® Competent E. coli were transformed. Plasmids were isolated using the QIAprep® spin midiprep kit from Qiagen GmbH according to the manufacturer's protocol. Cloned PCR products were verified by sequencing and compared to the published promoter sequences.

Standard curves material preparation: Plasmids were linearized by digestion with the restriction enzyme BamHI (Roche) and then purified using the QIAquick® PCR purification kit (Qiagen GmbH; according to the manufacturer's protocol). Concentratrions of PCR or plasmidic preparations were determined using UV spectrophotometry. A stock solution of 2×10⁷ copies/5 μl was prepared and stored at −80° C. until use. Dilutions of standard curves (2×10⁶-2×10¹ copies/5 μl) were freshly prepared for each experiment.

Real time MS-PCR: CTAG1B, CTAG2, PRAME and □-actin quantifications were performed by real-time MSP assays. These consisted of parallel amplification/quantification processes using specific primer combination for each Amplifluor® assay formats using either an ABI Prism® 7900HT instrument (Applied Biosystems) or i-Cycler (BioRad). Cycling conditions were: Stage1: 50° C. for 2 min, Stage2: 95° C. for 15 min, Stage3: 95° C. for 15 s, 57° C. for 30 s, 57° C. for 30 s (plateau-data collection) for 45 repeats. Results were generated using either the SDS 2.2.2 software from Applied Biosystems or the iCycler iQ version 3.1 software from BioRad, and exported as Ct values (cycle number at which the amplification curves cross the threshold value, set automatically by the software).

### Results:

Primers corresponding to the primer pairs, described in Table 1, were used to prepare PCR products for each assay. For this purpose, short region for CTAG1B_{—}1, CTAG2_{—}1 and PRAME_{—}7 as described in FIG. 1, FIG. 2 and FIG. 3 were amplified using bisulphite treated CpGenome™ Universal Unmethylated DNA as template material. For each primer pairs several annealing temperatures were tested (57° C. to 62° C.) in order to get a single PCR product. These PCR products were purified and quantified and then diluted to prepare standard material for each assays. Initial real time MS-PCR were prepared using the purified PCR products (specific to each assay) as template and different primer pair combinations as described in Table 1. Finally the following assays were retained for further investigation: CTAG1B_{—}1_S_AMP, CTAG1B_{—}2_AS_AMP, CTAG2_{—}1_S_AMP, CTAG2_{—}1_AS_AMP, CTAG2_{—}3_S_AMP, PRAME_{—}1_AS_AMP, PRAME_{—}2_S_AMP, PRAME_{—}3_AS_AMP, PRAME_{—}6_AS_AMP and PRAME_{—}7_S_AMP. An example of the performance (including all 6 data points) of each is shown in Table 2.

TABLE 2
Summary of slopes and PCR efficiencies CT assays

| Assay Name | Slope | R² | Efficiency |
|---|---|---|---|
| CTAG1B_1_S_AMP | −3.5841 | 0.9999 | 90.11% |
| CTAG1B_2_AS_AMP* | −3.5044 | 0.9919 | 92.91% |
| CTAG2_1_AS_AMP | −3.2955 | 0.9998 | 101.11% |
| CTAG2_1_S_AMP | −3.5534 | 0.9980 | 91.17% |
| CTAG2_3_S_AMP | −3.5971 | 1.0000 | 89.67% |
| PRAME_1_AS_AMP | −3.4750 | 0.9960 | 100.86% |
| PRAME_2_S_AMP | −3.5334 | 0.9993 | 91.87% |
| PRAME_3_AS_AMP | −3.7041 | 0.9990 | 86.20% |
| PRAME_6_AS_AMP | −3.5841 | 0.9999 | 90.11% |
| PRAME_7_S_AMP | −3.8679 | 0.9994 | 81.36% |

(*only 4 data points were included)

For some assays, the PCR product was cloned in a plasmid and the construct was used as material for the standard curve preparation instead of the PCR products.

### Example 3

### Concordance Between Real Time MS-PCR Assays and Quantitative Real Time RT-PCR Results for CTAG1B, CTAG2 and PRAME Genes

### Material and Methods:

RNA from cell lines, lung samples, melanoma samples and breast samples were extracted using the Tripure reagent (Roche, Vilvoorde, Belgium) according to the manufacturer's instructions except that the isopropanol precipitation was replaced by an RNeasy purification step (Qiagen, Venlo, Netherlands). RNA concentration was determined from the optical density value at 260 nm. Cell lines DNA was extracted using either “Puregene Cell and Tissue Kit” (Qiagen #158767) or “QIAamp DNA mini kit (Qiagen #51304)

Lung biopsy samples, provided by GSKBio in RNA Later® solution, were used to extract genomic DNA. About 10 mg of lung tissues were sliced into very small pieces using a razor blade and extraction was performed following the phenol extraction method. DNA from formalin fixed paraffin embedded (FFPE) breast tissues were extracted following the same phenol/chloroform method after Xylene treatment.

DNA from melanoma biopsy samples were extracted using either “QIAamp DNA mini kit” (Qiagen #51304) or “Maxwell 16 Tissue DBA purification kit” (Promege AS1030).

For xylene treatment: each tube containing the FFPE tissues was incubated at RT for 2 h with 750 μl of Xylene (Merck #1.08681.1000) to dissolve the paraffin. When the paraffin was totally dissolved 250 μl of 70% ethanol was added and after mixing, tubes were centrifuged for 15 min at maximum speed. The supernatant was removed and the samples were air dried at room temperature.

DNA was extracted from the sample with use of phenol/chloroform: briefly, samples were first incubated overnight with 50 to 100 μg/ml of proteinase K (Roche) and 1% SDS final concentration at 48° C., with shaking at 1100 rpm. 1 volume of Phenol: Chloroform: Isoamylalcohol (25:24:1) from Invitrogen was added to 1 volume of sample and the mixture was transferred to a Phase Lock Gel tube (Eppendorf). After thorough mixing, the tubes were centrifuged to separate the phases and recover the nucleic-acid-containing aqueous upper one. Extraction with Phase lock gel tubes was done once again. DNA was precipitated by addition 750 μl of an EtOH/NH4Ac solution and 2 μl of glycogen (Roche). It was then dissolved in 50 μl LoTE (3 mM TRIS, 0.2 mM EDTA, pH 8.0).

DNA was quantified using the Picogreen® dsDNA quantitation kit (Molecular Probes, #P7589) following the manufacturer's recommendations. λDNA provided with the kit was used to prepare a standard curve. The data were collected using a FluoStar Galaxy plate reader (BMG Lab technologies, Germany).

Bisulfite treatment: up to 1.5 μg of DNA in LoTE was treated using the EZ DNA Methylation kit from Zymo Research (#D5002), Briefly, aliquots of 45 μl were mixed with 5 μl of M-Dilution Buffer and incubated at 37° C. for 15 min shaking at 1100 rpm. Then 100 μl of the diluted CT Conversion Reagent was added and samples were incubated at 70° C. for 3 h, shaking at 1100 rpm in the dark. After conversion, the samples were further desalted and desulfonated according to manufacturer's instructions and elution was done in 50 μl Tris-HCl 1 mM Ph 8.0.

2.4 μl of bisulfite treated DNA is used in a 12 μl final reaction containing 100 nM final concentration of each primers (specific combination according to Table 1) and the 2× mix from the Quantitect Probe PCR Kit (Qiagen #204345). CpGenome™ Universal Methylated and Unmethylated DNA (Chemicon International, CA, USA; Cat.# S7821 and Cat.# S7822) were included in each run as negative and positive controls. Reactions were loaded in a 384 well plate. Assays were run on an Applied Biosystem 7900HT instrument. Cycling conditions were: Stage 1: 50° C. for 2 min, Stage 2: 95° C. for 10 min, Stage 3 (45 repeats): 95° C. for 15 sec, 57° C. for 30 sec (or 62° C. for □-actin), 57° C. for 30 sec (or 62° C. for □-actin) corresponding to the plateau-data collection. Results were generated using either the SDS 2.2.2 software from Applied Biosystems, and exported as Ct values (cycle numbers at which the amplification curves cross the threshold value, set automatically by the software). Ct values were used to calculate copy numbers based on a linear regression of the values plotted on a standard curve of 20-2×10̂6 gene copy equivalents. The ratio between the gene of interest and b-actin was calculated to generate the test result.

cDNA synthesis from 2 μg of total RNA was performed in a 20 μl mixture containing 1× first strand buffer, 0.5 mM of each dNTP, 10 mM of dithiothreitol, 20 U of rRNase inhibitor (Promega cat. N2511), 2 μM of oligo(dT) 15 and 200 U of M-MLV reverse transcriptase (Life Technologies cat. 28025-013) for 1 h30 at 42° C. cDNA corresponding to 50 ng of total RNA was amplified by PCR in a 25 μl mixture containing TaqMan buffer, 5 mM MgCl2, 0.4 mM dUTP, 0.2 mM of each nucleotide, 0.625 U of Ampli Taq Gold DNA polymerase, 0.05 U of UNG, 0.2 μM of each oligonucleotide primers and 0.2 μM of TaqMan MGB probe. Specific oligonucleotide primers and MGB probes were used. Target genes and beta-actin genes were amplified by quantitative PCR using TaqMan chemistry 7900 system (PE Applied Biosystems). The amplification profile was 1 cycle of 2 min at 50° C., 1 cycle of 12 min at 95° C. and 40 cycles of 15 s at 95° C. and 1 min at 60° C. The fluorescent signal generated by the degradation of the TaqMan probe was detected in real-time during all elongation steps at 60° C. Raw data were analysed using the real-time sequence detection software (Applied Biosystems, Warrington, UK). The ratio between the target gene and b-actin was calculated to generate the test result.

### Specifications:

For Real time MS-PCR the following specifications were applied to the standard curve: At least 4 points in duplicate; □Ct (between duplicates)<1.5; PCR efficiency >80%; R2>0.99; Positive controls have to be positive; Negative controls have to be negative.

The criteria for results interpretation were based on Ratio values calculated with the standard equivalent copies of the gene of interest and the b-actin copie: ratio=(gene of interest copies/β-actin copies) X 1000.

For gene expression, specifications were that NTC had Ct greater than 35 and positive control was positive with ΔCt duplicates for target gene and beta-actin <2.

Gene expression results are calculated relatively to actin expression. The cut-off for PRAME expression, is set at 1E−04 copy/actin copy and for CTAG1B and CTAG2, the cut-off is set at 1E−05 copy/actin copy. Samples were valid if beta-actin Ct was lower than 23 and ΔCt duplicates <2 and if target gene ΔCt duplicates was <2.

TABLE 3
Criteria for Real time MS-PCR results interpretation.

| | | Methyaltion/ |
|---|---|---|
| Results | | unmethylation status |
| β-actin < 2.00 copies | | Result INVALID |
| 2.00 < β-actin < 200.00 copies and CT | | Result INVALID |
| gene < 2.00 copies | | |
| β -actin ≧ 200.00 copies and | | Result METHYLATED |
| CT gene < 2.00 copies | | (no ratio) |
| β -actin ≧ 2.00 | Ratio: CT gene./β - | Result UNMETHYLATED |
| copies and | actin × 1000 ≧ cut-off | |
| CT gene ≧ 2.00 | value* | |
| copies | | |
| β -actin ≧ 2.00 | Ratio: CT gene./β - | Result METHYLATED |
| copies and CT | actin × 1000 < cut-off | |
| gene ≧ 2.00 copies | value* | |

*The cut-off value is set specifically for each assay.

### Results

### Cell Lines

After selection of the most optimal real-time MS-PCR conditions using alternative standard curve material, various cell lines were assayed for CTAG1B, CTAG2 and PRAME demethylation assays. Results were compared to the ones obtained using real time RT-PCR. One assay is shown for each gene studied (CTAG1B, CTAG2, PRAME) see Table 4, Table 5 and Table 6.

TABLE 4
Summary of real time MS-PCR results obtained
with the CTAG1B_1_S assay versus real
time RT-PCR data (expression) for 20 cell lines;
result display sorted by ratio value.

| | Ratio | |
|---|---|---|
| | CTAG1B_1_S/ | |
| | b-actin | Expression |
| | (copies) X | data |
| Cell line | 1000 | CTAG1B |
| HL-60 | 0 | NA |
| SW-480 | 0 | Negative |
| LNCap | 1.81 | Negative |
| LS-174T | 28.17 | Negative |
| SW620 | 73.75 | Negative |
| HT29 | 74.07 | Negative |
| MCF7 | 81.95 | Negative |
| KG1 | 85.54 | Negative |
| NCI-H460 | 93.52 | Negative |
| Staq | 94.33 | Negative |
| Gerl | 99.43 | Negative |
| T-47D | 116.29 | Negative |
| UACC3199 | 120.74 | NA |
| PC3 | 140.68 | Negative |
| SK-MEL-5 | 165.07 | Negative |
| CRL5815 | 180.51 | Negative |
| SKOV3 | 196.44 | Negative |
| K562 | 204.86 | Negative |
| CRL2505 | 297.39 | 2.03E−02 |
| CRL5803 | 390.78 | 1.71E−02 |

Expression data N/A: not applicable; Negative: Not expressing samples; Expressing samples are indicated by the corresponding value.

TABLE 5
Summary of real time MS-PCR results obtained with
CTAG1B_1_AS assay versus real time RT-PCR data (expression)
for 20 cell lines; result display sorted by ratio value.

| | Ratio | |
|---|---|---|
| | CTAG2_1_AS/ | |
| | b-actin | Expressoion |
| | (copies) X | data |
| Cell line | 1000 | CTAG2 |
| CRL5803 | 0 | Negative |
| Gerl | 0 | Negative |
| KG1 | 0 | Negative |
| Staq | 0 | Negative |
| SK-MEL-5 | 0 | [4.75E−05] |
| HL-60 | 0 | NA |
| T-47D | 0 | Negative |
| PC3 | 0 | Negative |
| UACC3199 | 1.11 | NA |
| NCI-H460 | 1.36 | Negative |
| SW-480 | 1.77 | Negative |
| SW620 | 1.94 | Negative |
| MCF7 | 5.8 | Negative |
| LNCap | 8.96 | [2.23E−05] |
| HT29 | 10.15 | Negative |
| K562 | 23.4 | 3.93E−03 |
| SKOV3 | 28.73 | Negative |
| CRL5815 | 52.52 | 2.53E−03 |
| CRL2505 | 153.86 | 3.63E−02 |
| LS-174T | 169.91 | 2.13E−03 |

Expression data N/A: not applicable; Negative: Not expressing samples; Expressing samples are indicated by the corresponding value; borderline samples in bracket.

TABLE 6
Summary of real time MS-PCR results obtained
with the PRAME_3_AS assay versus real
time RT-PCR data (expression) for 20 cell lines;
results display sorted by ratio value.

| | Ratio | |
|---|---|---|
| | PRAME_3_AS/ | |
| | b-actin | Expressoion |
| | (copies) X | data |
| Cell line | 1000 | PRAME |
| KG1 | 1.79 | 2.75E−05 |
| HT29 | 9.07 | Negative |
| MCF7 | 9.2 | Negative |
| SW- | 24.65 | Negative |
| 480_G1 | | |
| SW620 | 99.84 | 1.44E−05 |
| NCI-H460 | 116.05 | 2.65E−03 |
| UACC3199 | 120.77 | NA |
| CRL2505 | 150.82 | 1.97E−03 |
| T-47D | 167.05 | 1.83E−03 |
| LS- | 187.35 | Negative |
| 174T_G1 | | |
| PC3 | 190.74 | 3.48E−03 |
| CRL5815 | 233.9 | 8.38E−05 |
| CRL5803 | 251.42 | 2.99E−02 |
| SK-MEL-5 | 271.66 | 1.11E−01 |
| Staq | 292.37 | Positive |
| Gerl | 461.68 | Positive |
| LNCap | 487.28 | 2.36E−02 |
| HL-60_G1 | 588.05 | NA |
| SKOV3 | 659.56 | 9.69E−03 |
| K562 | 922.78 | 2.71E−01 |

Expression data N/A: not applicable; Negative: Not expressing samples; Expressing samples are indicated by the corresponding value.

One aim of this experiment was to find suitable positive and negative cell lines for assays processed on cancer samples. When real time MS-PCR data are compared to expression data, it was observed (Table 4) that CRL2505 and CRL5803 present the highest CTAG1B_{—}1_S/b-actin ratios and could therefore be used as positive cell lines for CTAG1B demethylation when using the CTAG1B_{—}1_S assay. SW-480 and HL-60 cell lines can be used as negative cell lines for this assay.

It was also possible to define the best positive and negative cell line for the CTAG2 and PRAME assay tested (Table 7 and Table 8)

TABLE 7
Best positive cell line(s) and negative cell
line(s) for CTAG2 demethylation assays

| Assay | Best positive cell line(s) | Negative cell line(s) |
|---|---|---|
| CTAG2_1_S | CRL2505 | CRL5803/GERL |
| CTAG2_1_AS | CRL2505/LS-174-T | CRL5803/GERL |
| CTAG2_2_S | CRL5815/K562 | CRL5803 |
| CTAG2_3_S | K562 | CRL5803/GERL |

TABLE 8
Best positive cell line(s) and negative cell
line(s) for PRAME demethylation assays

| Assay | Best positive cell line(s) | Negative cell line(s) |
|---|---|---|
| PRAME_1_AS | HL60/LnCap | MCF7 |
| PRAME_2_S | K562 | MCF7/HT29 |
| PRAME_3_AS | K562 | KG1/MCF7 |
| PRAME_6_AS | K562 | KG1/MCF7 |
| PRAME_7_S | GERL | MCF7/HT29 |

### Non-Small Cell Lung Cancer (NSCLC)

51 NSCLC samples were used to study CTAG1B, CTAG2 and PRAME expression by real time RT-PCR as well as their methylation status by real time MS-PCR. 3 samples were found invalid for at least one technique and therefore results obtained for 48 NSCLC were compared. Result Comparisons were expressed graphically. No good concordance was observed for CTAG1B with the CTAG1B_{—}1_S assay (FIG. 6). When comparing CTAG2 expression to results obtained with 4 demethylation assays, the best concordance was obtained with CTAG2_{—}1S (FIG. 8) and CTAG2_{—}1AS assays (FIG. 7), showing 87.5% concordance between both techniques. CTAG2_{—}2_S (FIG. 9) and CTAG2_{—}3_S (FIG. 10), show lower concordance with 68.8% and 66.7% respectively. About PRAME expression, a range of concordance is observed from 66.7% to 85.4% depending on the assay (FIG. 11, FIG. 12, FIG. 13, FIG. 14 and FIG. 15). The best concordance with expression data was obtained with PRAME_{—}3_AS demethylation assay (FIG. 14), showing 85.4% concordance. Table 9 show a summary of all the results obtained when comparing gene expression and gene methylation in NSCLC samples.

TABLE 9
Result summary displaying the Pearson correlation
coefficients (R) and concordance results for Non-
small Cell Lung Cancer (NSCLC) samples.

| Assay | R | Concordance |
|---|---|---|
| CTAG1B_1_S | 0.35 | / |
| CTAG2_1_S | 0.88 | 87.5% |
| CTAG2_1_AS | 0.86 | 87.5% |
| CTAG2_2_S | 0.47 | 68.8% |
| CTAG2_3_S | 0.55 | 66.7% |
| PRAME_1_AS | 0.39 | 66.7% |
| PRAME_2_S* | 0.64 | 79.2% |
| PRAME_3_AS | 0.73 | 85.4% |
| PRAME_6_AS | 0.24 | 66.7% |
| PRAME_7_S | 0.62 | 83.3% |

*PRAME_2_S standard curve did not meet the specifications

### Melanoma Samples

DNA and RNA were extracted from 31 melanoma tissue samples in RNA Later® solution. Similarly to what was done when studying NSCLC, these melanoma samples were used to study CTAG1B, CTAG2 and PRAME expression by real time RT-PCR as well as their methylation status by real time MS-PCR. Like for NSCLC, no good concordance was observed with expression data when using CTAG1B_{—}1_S demethylation assay. For CTAG2 gene, the best concordance was observed with 3 assays: CTAG2_{—}1_S (FIG. 18), CTAG2_{—}1_AS (FIG. 17) and CTAG2_{—}3S (FIG. 20) assays, all displaying 74.2% concordance between both techniques. For PRAME gene, all PRAME assays (FIG. 21, FIG. 22, FIG. 23, FIG. 24 and FIG. 25) showed a good concordance (90.3% and 93.5%). However, only PRAME_{—}7_S (Table 9) displayed an acceptable R Pearson correlation coefficient, possibly due to the low number of negative samples (2) in that set. Results of concordance and correlation obtained on Melanoma samples are summarized in Table 10.

TABLE 10
Result summary displaying the Pearson correlation coefficients
(R) and concordance results for Melanoma samples

| Assay | R | Concordance |
|---|---|---|
| CTAG1B_1_S | 0 | / |
| CTAG2_1_S | 0.62 | 74.2% |
| CTAG2_1_AS | 0.63 | 74.2% |
| CTAG2_2_S | 0.34 | 51.6% |
| CTAG2_3_S | 0.58 | 74.2% |
| PRAME_1_AS | 0.26 | 93.5% |
| PRAME_2_S | 0.29 | 90.3% |
| PRAME_3_AS | 0.48 | 90.3% |
| PRAME_6_AS | 0.51 | 90.3% |
| PRAME_7_S | 0.70 | 90.3% |

### Breast Cancer Samples

29 breast cancer samples were used to study CTAG2 expression by real time RT-PCR as well as its methylation status by real time MS-PCR. 1 sample was found invalid for at least one technique and therefore results obtained for 28 breast cancer samples were compared. Results obtained for CTAG2_{—}1_AS and CTAG2_{—}3_S assays were respectively, 71.4% (FIG. 26) and 78.6% concordance (FIG. 27).

### CONCLUSION

Cancer/testis (CT) antigens which are expressed almost exclusively in testis in normal tissues, are found to be expressed in cancer tissues else than testis. Expression of CT genes can be studied by real time RT-PCR as shown in this study but also indirectly by studying the gene promoter (or close by region) methylation status. Indeed abnormal expression could be related to hypomethylation. Study on cell lines have provided positive and negative control cell lines for all assays including CTAG1B demethyaltion assay. In NSCLC, melanoma and breast cancer tissues, except for the CTAG1B assays, it was possible to show some concordance between gene expression and gene hypomethylation status. The best concordance for CTAG2 expression were obtained with CTAG2_{—}1_S and CTAG2_{—}1_AS demethylation assays, both showing 87.5% concordance within NSCLC samples and 74.2% in melanoma samples. While CTAG2_{—}3_S assay was showing 78.6% among breast cancer samples. Concerning PRAME, the best concordance was obtained with PRAME_{—}3_AS demethylation assay in NSCLC (85.4%) and PRAME_{—}1_AS assay in melanoma (93.5%). Two assays were performed on breast cancer tissues: CTAG2_{—}1_AS and CTAG2_{—}3_S showing respectively, 71.4% and 78.6% concordance.

### Example 4

### Diagnosis of Non-Small Cell Lung Cancer (NSCLC) with MAGE-A3 Hypomethylation Assays

### Material and Methods:

DNA from fine needle biopsies (FNB) were extracted.

Similar to example 3, each sample and cell line DNA was assayed for DNA amount using Picogreen® dsDNA quantitation kit (Molecular Probes, #P7589) following the manufacturer's recommendations. Data were collected and analysed with a FluoStar Galaxy plate reader (BMG Lab technologies, Germany). As previously described, up to 1.5 μg of DNA were treated using the EZ DNA Methylation kit from Zymo Research (see example 2 for details), elution was done in 25 μl. Then 2.4 μl of Bisulfite Treated DNA were processed, in a 12 μl total reaction volume containing 100 nM final concentration of each primer, through β-Actin (same as for example 3) and MAGE-A3 (MAGE-A3 GO_{—}2 U) Real Time MSP assays (Table 1). The PCR mix used is the “iTaq supermix” with Rox from Bio-Rad (#172-5855). A plasmidic construct (cloned PCR product in a TOPO®

Cloning vector from Invitrogen life technologies) was used to prepare the standards. Reactions were loaded in a 384 well plate. Assays were run on an Applied Biosystem 7900HT instrument. Cycling conditions for β-Actin assay were as follows: stage 1, 50° C. for 2 minutes; stage 2, 95° C. for 10 minutes; stage 3, 95° C. for 15 s then 62° C. for 1 min (plateau-data collection) for 45 repeats. Cycling conditions for MAGE-A3 GO_{—}2 U assay were as follow: stage 1, 50° C. for 2 minutes; stage 2, 95° C. for 10 minutes; stage 3, 95° C. for 15 s then 59° C. for 1 min (plateau-data collection) with 45 repeats. Results were generated using the SDS 2.2.2 software from Applied Biosystems, and exported as Ct values (cycle numbers at which the amplification curves cross the threshold value, set automatically by the software). Ct values were used to calculate copy numbers based on a linear regression of the values plotted on a standard curve of 20-2×10̂6 gene copy equivalents. The ratio between the gene of interest and β-actin was calculated to generate the test result.

### Results

Hereby are described results obtained with Fine needle biopsies (FNB) from Non-Small Cell Lung Cancer (NSCLC) patients with advanced disease who do not undergo resection of the tumor and for which no expression data could be obtained.

After DNA extraction and conversion (bisulfite treatment), 35 FNB were processed through the MAGE-A3 GO_{—}2 U hypomethylation assay (Table 1). The same criteria for result interpretation applied as for example 3 (Table 3). As it was not possible to compare the hypomethylation test to qRT-PCR, cut-off for the hypomethylation test had to be established indirectly. Based on results obtained with the same assay on formalin fixed paraffin embedded (FFPE) lung tissues and tissues in RNA later solution from stage IB and II NSCLC patients, the chosen cut-off is 112. The corresponding results are presented in Table 11. It should be noticed that the cut-off value could be adjusted to better correspond to expression data.

TABLE 11
Methylation status of 35 fine needle biopsies (FNB)
from Non-Small Cell Lung Cancer (NSCLC) patients.

| | | Ct | MAGE- | Ratio | |
|---|---|---|---|---|---|
| | | (MAGE-A3 | A3 | gene/b-actin | METHYLATION |
| Detector | Sample | GO_2 U assay) | Copies | (copies) X 1000 | STATUS |
| MAGEA3 | 233057_G1 | 33.75 | 68.85 | 37.19 | |
| MAGEA3 | 233058_G1 | 31.05 | 359.64 | 262.90 | UNMETHYLATED |
| MAGEA3 | 233059_G1 | 36.79 | 10.71 | 5.91 | |
| MAGEA3 | 233060_G1 | 34.22 | 51.78 | 24.29 | |
| MAGEA3 | 233061_G1 | 34.07 | 56.49 | 255.84 | UNMETHYLATED |
| MAGEA3 | 233062_G1 | 30.70 | 444.05 | 215.36 | UNMETHYLATED |
| MAGEA3 | 233063_G1 | 34.70 | 38.49 | 30.64 | |
| MAGEA3 | 233064_G1 | 26.07 | 7549.74 | 442.02 | UNMETHYLATED |
| MAGEA3 | 233065_G1 | 34.54 | 42.55 | 70.62 | |
| MAGEA3 | 233066_G1 | 36.12 | 16.10 | 2.62 | |
| MAGEA3 | 233067_G1 | 36.73 | 11.10 | 12.36 | |
| MAGEA3 | 233068_G1 | 39.29 | 2.32 | 0.84 | |
| MAGEA3 | 233069_G1 | Undetermined | 0.00 | 0.00 | |
| MAGEA3 | 233070_G1 | 33.99 | 59.43 | 16.77 | |
| MAGEA3 | 233071_G1 | Undetermined | 0.00 | 0.00 | |
| MAGEA3 | 233072_G1 | Undetermined | 0.00 | 0.00 | |
| MAGEA3 | 233073_G1 | 30.79 | 422.20 | 561.79 | UNMETHYLATED |
| MAGEA3 | 233074_G1 | 33.74 | 69.27 | 47.80 | |
| MAGEA3 | 233075_G1 | 29.59 | 879.02 | 554.36 | UNMETHYLATED |
| MAGEA3 | 233076_G1 | Undetermined | 0.00 | 0.00 | |
| MAGEA3 | 233077_G1 | 29.41 | 980.70 | 186.65 | UNMETHYLATED |
| MAGEA3 | 233078_G1 | 28.64 | 1567.01 | 609.72 | UNMETHYLATED |
| MAGEA3 | 233079_G1 | 36.24 | 15.04 | 0.96 | |
| MAGEA3 | 233080_G1 | 31.40 | 290.03 | 245.69 | UNMETHYLATED |
| MAGEA3 | 233081_G1 | 29.53 | 908.41 | 230.92 | UNMETHYLATED |
| MAGEA3 | 233082_G1 | 32.43 | 154.15 | 45.57 | |
| MAGEA3 | 233083_G1 | 31.76 | 233.19 | 162.65 | UNMETHYLATED |
| MAGEA3 | 233084_G1 | 36.01 | 17.32 | 145.84 | UNMETHYLATED |
| MAGEA3 | 233085_G1 | 36.22 | 15.15 | 61.27 | |
| MAGEA3 | 233086_G1 | 31.28 | 311.56 | 213.53 | UNMETHYLATED |
| MAGEA3 | 233087_G1 | 26.57 | 5583.40 | 338.05 | UNMETHYLATED |
| MAGEA3 | 233088_G1 | 32.09 | 190.33 | 124.20 | UNMETHYLATED |
| MAGEA3 | 233089_G1 | 34.18 | 52.80 | 25.14 | |
| MAGEA3 | 233090_G1 | 36.18 | 15.57 | 1.50 | |
| MAGEA3 | 233091_G1 | 34.78 | 36.55 | 269.73 | UNMETHYLATED |
| MAGEA3 | Lncap_a_G1 | 30.23 | 592.88 | 1995.86 | UNMETHYLATED |
| MAGEA3 | Lncap_b_G1 | 29.93 | 714.43 | 2616.01 | UNMETHYLATED |
| MAGEA3 | DU145_a_G1 | Undetermined | 0.00 | 0.00 | |
| MAGEA3 | DU145_b_G1 | 41.93 | 0.00 | 0.00 | |

Lncap is a cell line used as a positive control for the hypomethylation test while DU145 is a cell line used as negative control for this assay. Analysis was done using a cut-off of 112 for the calculated ratio.

By setting the cut-off value at 112 then the percentage of patients with hypomethylation of MAGE-A3 is 45.7%. These patients are potentially expressing MAGE-A3.

### CONCLUSION

A demethylation/hypomethylation assay would be of particular interest in the case of sample types for which qRT-PCR prove to be difficult. This is the case of Fine needle biopsies (FNB). As this technique is non invasive it could be in the future extended to other NSCLC patients.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all embodiments described herein are considered to be broadly applicable and combinable with any and all other consistent embodiments, as appropriate.

Various publications are cited herein, the disclosures of which are incorporated by reference in their entireties.

1. An oligonucleotide, primer or probe comprising or consisting essentially of or consisting of the nucleotide sequence of any of SEQ ID NO. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 44, 61, 62 or 63 which oligonucleotide, primer or probe is useful for the detection of the methylation status of a gene.

2. An oligonucleotide, primer or probe according to claim 1 comprising, consisting essentially of or consisting of the nucleotide sequence of any of SEQ ID NO. 9 to 12 and SEQ ID NO. 21 to 40 which oligonucleotide, primer or probe is useful for the detection of the methylation status of a gene.

3. An oligonucleotide, primer or probe according to claim 1 comprising or consisting essentially of or consisting of the following contiguous sequences in 5′ to 3′ order:
(a) a first nucleotide sequence of between approximately 6 and 30 nucleotides, wherein a nucleotide within said first nucleotide sequence is labelled with a first moiety selected from the donor moiety and the acceptor moiety of a molecular energy transfer pair, wherein the donor moiety emits fluorescence at one or more particular wavelengths when excited, and the acceptor moiety absorbs and/or quenches said fluorescence emitted by said donor moiety;
(b) a second, single-stranded nucleotide sequence comprising, consisting essentially of or consisting of between approximately 3 and 20 nucleotides;
(c) a third nucleotide sequence comprising, consisting essentially of or consisting of between approximately 6 and 30 nucleotides, wherein a nucleotide within said third nucleotide sequence is labelled with a second moiety selected from said donor moiety and said acceptor moiety, and said second moiety is the member of said group not labelling said first nucleotide sequence, wherein said third nucleotide sequence is complementary in reverse order to said first nucleotide sequence such that a duplex can form between said first nucleotide sequence and said third nucleotide sequence such that said first moiety and second moiety are in proximity such that, when the donor moiety is excited and emits fluorescence, the acceptor moiety absorbs and quenches said fluorescence emitted by said donor moiety; and
(d) at the 3′ end of the primer, a fourth, single-stranded nucleotide sequence comprising, consisting essentially of or consisting of between approximately 8 and 40 nucleotides that comprises or consists essentially of or consists of at its 3′ end a sequence of any of SEQ ID NO. 2, 4, 6, 8, 10, 12, 14, 16, 18, 20, 22, 24, 26, 28, 30, 32, 34, 36, 38, 40, 42, 44, 61 or 63 (and able to prime synthesis by a nucleic acid polymerase of a nucleotide sequence complementary to a nucleic acid strand comprising the portion of the unmethylated DNA of the CTAG1B, CTAG2, MageA3 and/or PRAME gene);
wherein when said duplex is not formed, said first moiety and said second moiety are separated by a distance that prevents molecular energy transfer between said first and second moiety.

4. Primer pair comprising a primer according to claim 1.

5. Primer pair comprising a primer according to claim 3.

6. Primer pair comprising or consisting essentially of or consisting of the nucleotide sequence of SEQ ID NO. 9 and SEQ ID NO. 10; SEQ ID NO. 11 and SEQ ID NO. 12; SEQ ID NO. 21 and SEQ ID NO. 22; SEQ ID NO. 23 and SEQ ID NO. 24; SEQ ID NO. 25 and SEQ ID NO. 26; SEQ ID NO. 27 and SEQ ID NO. 28; SEQ ID NO. 29 and SEQ ID NO. 30; SEQ ID NO. 31 and SEQ ID NO. 32; SEQ ID NO. 33 and SEQ ID NO. 34; SEQ ID NO. 35 and SEQ ID NO. 36; SEQ ID NO. 37 and SEQ ID NO. 38; SEQ ID NO. 39 and SEQ ID NO. 40; or SEQ ID NO. 61 and 62.

7. A kit for detecting the methylation status of a gene comprising at least one oligonucleotide, primer or probe as defined in claim 1.

8. An oligonucleotide, primer or probe as defined in claim 1, wherein said gene is the CTAG1B, CTAG2, MageA3 and/or PRAME gene.

9. Method of detecting the presence and/or amount of unmethylated CTAG1B, CTAG2, MageA3 and/or PRAME gene in a DNA-containing sample, comprising:
(a) contacting/treating the DNA-containing sample with a reagent which selectively modifies unmethylated cytosine residues in the DNA to produce detectable modified residues but which does not modify methylated cytosine residues
(b) amplifying at least a portion of the unmethylated gene of interest using at least one primer pair, at least one primer of which is designed to bind only to the sequence of unmethylated DNA following treatment with the reagent.

10. Method according to claim 9 wherein at least one primer in the primer pair comprises, consists essentially of, or consists of the nucleotide sequence of any of SEQ ID NO. 1 to 40 or SEQ ID NO. 61 to 63.

11. Method of diagnosing cancer or predisposition to cancer comprising detecting the methylation status of at least one of the CTAG1B, CTAG2, MageA3 and/or PRAME gene in a sample by using an oligonucleotide, primer or probe as defined in claim 1, wherein the presence of unmethylated CTAG1B, CTAG2, MageA3 and/or PRAME in the sample is indicative for cancer or predisposition to cancer.

12. Method for identifying and/or selecting a patient suitable for treatment with a CTAG1B, CTAG2, MageA3 and/or PRAME immunotherapeutic comprising detecting the methylation status of the CTAG1B, CTAG2, MageA3 and/or PRAME gene in a sample of the patient by using an oligonucleotide, primer or probe as defined in claim 1, wherein if the CTAG1B, CTAG2, MageA3 and/or PRAME gene is unmethylated the subject is identified and/or selected for treatment with the CTAG1B, CTAG2 MageA3 and/or PRAME immunotherapeutic.

13. Method for predicting the likelihood of successful treatment of cancer comprising detecting the methylation status of the CTAG1B, CTAG2, MageA3 and/or PRAME gene in a sample of the patient by using an oligonucleotide, primer or probe as defined in claim 1, wherein if the gene is unmethylated the likelihood of successful treatment with a CTAG1B, CTAG2, MageA3 and/or PRAME immunotherapeutic is higher than if the gene is methylated.

14. Method of selecting a suitable treatment regimen for cancer comprising detecting the methylation status of the CTAG1B, CTAG2, MageA3 and/or PRAME gene in a sample of the patient by using an oligonucleotide, primer or probe as defined in claim 1, wherein if the gene is unmethylated, an immunotherapeutic is selected for treatment.

15. Method of treating cancer in a subject comprising administration of a composition comprising or encoding CTAG1B, CTAG2, MageA3 and/or PRAME wherein the subject has been selected for treatment on the basis of measuring the methylation status of a CTAG1B, CTAG2 MageA3 and/or PRAME gene by using an oligonucleotide, primer or probe as defined in claim 1.

16. Method of treating a patient comprising: measuring the methylation status of a CTAG1B, CTAG2, MageA3 and/or PRAME gene by using an oligonucleotide, primer or probe as defined in claim 1, and then administering to the patient a composition comprising or encoding CTAG1B, CTAG2, MageA3 and/or PRAME.

17. Method of treating a patient susceptible to recurrence of a CTAG1B, CTAG2, MageA3 and/or PRAME expressing tumour, the patient having been treated to remove tumour tissue, the method comprising: measuring the methylation status of a CTAG1B, CTAG2, MageA3 and/or PRAME gene in the tumour tissue by using an oligonucleotide, primer or probe as defined in claim 1, and then administering to the patient a composition comprising or encoding CTAG1B, CTAG2, MageA3 and/or PRAME.

18. Use of a composition comprising or encoding CTAG1B, CTAG2, MageA3 and/or PRAME in the manufacture of a medicament for the treatment of a patient suffering from a tumour, in which the patient has been selected for treatment on the basis of measuring the methylation status of a CTAG1B, CTAG2, MageA3 and/or PRAME gene, by using an oligonucleotide, primer or probe as defined in claim 1.

19. Use of a composition comprising or encoding CTAG1B, CTAG2, MageA3 and/or PRAME in the manufacture of a medicament for the treatment of a patient susceptible to recurrence of a CTAG1B, CTAG2, MageA3 and/or PRAME expressing tumour, in which a patient has been selected for treatment on the basis of measuring the methylation status of a CTAG1B, CTAG2, MageA3 and/or PRAME gene by using an oligonucleotide, primer or probe as defined in claim 1.

20. A method or use according to claim 16, in which the composition comprises CTAG1B, CTAG2, MageA3 and/or PRAME comprises full length CTAG1B, CTAG2, MageA3 and/or PRAME, substantially full-length CTAG1B, CTAG2, MageA3 and/or PRAME or fragments of CTAG1B, CTAG2, MageA3 and/or PRAME, for example peptides of CTAG1B, CTAG2, MageA3 and/or PRAME.

21. Method or use according to claim 20, in which CTAG1B is a fragment of full length CTAG1B including binding motifs to HLA-A2, selected from peptides 157-167, 157-165 and 155-163,

22. Method or use according to claim 20 wherein CTAG1B is a fragment of full length CTAG1B including one or more MHC Class 1 or Class 2 epitopes selected from A31, DR1, DR2, DR4, DR7, DP4, B35, B51, Cw3, Cw6 and A2 as disclosed in WO2008/089074.

23. Method or use according to claim 15, in which the CTAG1B, CTAG2 and/or PRAME protein, fragment or peptide is linked to a fusion partner protein.

24. A method or use according to claim 23, in which the fusion partner protein is protein D, a surface protein of the gram-negative bacterium, Haemophilus influenza B or a derivative thereof.

25. A method or use according to claim 23, in which the fusion partner protein is LytA or a derivative thereof comprising or consisting of the repeat portion of the LytA molecule found in the C terminal end starting at residue 178, or comprising residues 188-305.

26. A method or use according to claim 23, in which the fusion partner protein is NS1 (hemagglutinin), or a derivative thereof comprising the N terminal 81 amino acids of NS1.

27. A method or use according to claim 15, in which the composition comprises a nucleic acid molecule encoding the CTAG1B, CTAG2 and/or PRAME protein, fragment or peptide or fusion protein thereof.

28. A method or use according to claim 27, in which the nucleic acid molecule is provided within an expression vector.

29. A method or use according to claim 15, in which the CTAG1B, CTAG2 and/or PRAME protein, fragment or peptide containing composition, or nucleirc acid containing composition, further comprises one or more of an adjuvant, an immunostimulatory cytokine and a chemokine.

30. A method or use according to claim 29, in which the adjuvant comprises one or more of monophosphoryl lipid A or a derivative thereof, a saponin or a derivative thereof and a TLR9 antagonist.

31. A method or use according to claim 30, in which the TLR9 agonist is a CpG-containing oligonucleotide.

32. A method or use according to claim 29, in which the adjuvant is formulated in an oil-in-water emulsion or a water in oil emulsion, optionally containing cholesterol and/or tocopherol, or is formulated in a liposomal composition.

33. A method according to claim 9, in which the gene is the MageA3 gene and the sample or tumor tissue is taken by fine needle biopsy.
